(19) United States
(12) Patent Application Publication
(10) Pub. No.: US 2025/0133949 A1
(43) Pub. Date: Apr. 24, 2025

(54) ORGANIC OPTOELECTRONIC COMPOUND AND ORGANIC OPTOELECTRONIC DEVICE USING THE SAME

(71) Applicant: Raynergy Tek Incorporation, Hsinchu (TW)

(72) Inventors: Cheng-Chang LAI, Hsinchu (TW); Chuang-Yi LIAO, Hsinchu (TW)

(21) Appl. No.: 18/918,119

(22) Filed: Oct. 17, 2024

Related U.S. Application Data
(60) Provisional application No. 63/544,793, filed on Oct. 19, 2023.

(51) Int. Cl. H10K 85/10; H10K 85/40; H10K 85/60

(52) U.S. Cl. CPC H10K 85/113; H10K 85/40; H10K 85/6576

(57) ABSTRACT

An organic optoelectronic compound comprises a structure such as Formula I:

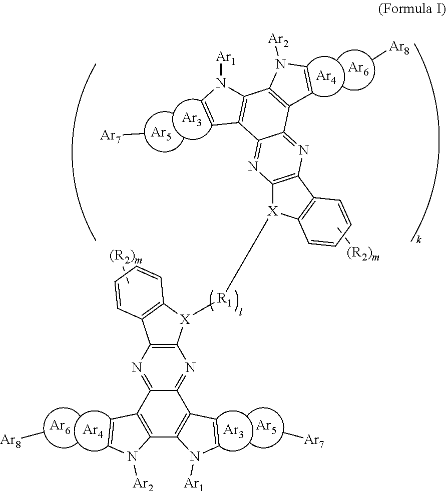

The organic optoelectronic compound features a pyrazine core structure, providing an additional conjugated plane, enhancing intermolecular interactions for smoother charge carrier flow and reducing energy loss, and improving thermal stability. Furthermore, by modifying the functional groups and structural symmetry, the arrangement of the material and its energy levels can be altered. The invention also provides an active layer material containing the compound and an organic optoelectronic device, both exhibiting good thermal stability.

The present application is based on, and claims priority from, America U.S. provisional patent application No. 63/544,793, filed on 2023 Oct. 19, and the disclosure of which is hereby incorporated by reference herein in its entirety.

### BACKGROUND OF THE INVENTION

### Field of the Invention

The present invention relates to an organic optoelectronic compound applied in an organic optoelectronic device, and in particular to an organic optoelectronic compound with a pyrazine core structure and an organic optoelectronic device comprising the same.

### Description of the Prior Art

In recent years, due to the vigorous development of solar energy and photodetector-related industries, the main applicable fields range from general livelihood and industries to medical testing and other purposes. In the application part of photodetectors, the increasing reliance on consumer electronics such as smartphones for applications like electronic payments and the expanding use of biometric technologies such as full-screen fingerprint recognition and vein pattern recognition has directly or indirectly driven the demand for such technologies. At present, many manufacturers have stepped up their deployment of photodetector-related technologies, and the future is promising.

As mentioned above, image detectors have become one of the fastest growing semiconductor product categories in recent years. Its applications can be mainly classified according to the photo detective band. For example, lenses, optical communications, biochips or fingerprint scanning all rely on photodetection technology in the visible band. Currently, the mainstream technology in the market is photodetectors composed of single crystal silicon. However, since the wavelength range occupied by visible light is far smaller than that of invisible region, the application demand for invisible region has been becoming a significantly increasing trend in the near future; for example, biometric technologies, such as vein scanners, iris sensors, and facial recognition, as well as the physiological vital sign monitoring technology of pulse oximetry measurement, and machine vision applications like LiDAR and time of flight sensors that are currently required by self-driving cars. Therefore, how to provide an organic photodetector with high performance and low cost in the absorption range of near infrared or shortwave infrared corresponding to the above applications is a very important issue at present.

Active layer materials play an important role in organic photodetectors and will directly affect device performance. The active layer material is divided into two parts: donor materials and acceptor materials. For the donor materials, the development of D-A conjugated polymers is the mainstream. The electron push-pull effect of electron-rich units and electron-deficient units in conjugated polymers can be used to control the energy levels and energy band gaps of polymers. The acceptor materials blended with the donor materials are usually fullerene derivatives with high conductivity, and its light absorption range is about 400˜600 nm. However, the structure of fullerene derivatives is not easy to be adjusted, and their light absorption and energy levels are limited within a certain range, which limits the overall combination of the donor materials and the acceptor materials. With the development of the market, the demand for materials in the near-infrared region is gradually increasing. Even if the light absorption range of the conjugated polymer of the donor materials can be adjusted to the near-infrared region, it may not be able to have a good match due to the limitation of fullerene acceptor materials. Therefore, it is very important to develop non-fullerene acceptor materials to replace traditional fullerene acceptor materials in the breakthrough of active layer materials. The material development of the non-fullerene acceptor materials is mainly to form A-D-A ladder type molecules with electron-rich centers and electron-deficient units on both sides. D is usually a ladder type molecule composed of benzene and thiophene, and A is usually an indanone-cyano derivative. ITIC is a representative non-fullerene acceptor with an absorption range about 600˜750 nm, and it also has good performance in organic photodetectors. In addition to the A-D-A ladder molecules, in 2019, the ladder type molecules published by Yang et.al with the A-D-A′-D-A structure, such as Y6 (BTP-4F), have a light absorption range of 600˜900 nm, which further extends the light absorption spectrum of non-fullerene acceptors to near-infrared region. However, the current non-fullerene acceptors with Y6 structure is served as the core suffer from insufficient thermal stability. For example, Y6, which performs better in the BTP series, has a glass transition temperature of only 102° C. and a decomposition temperature of only 318° C., which presents a significant challenge in terms of product lifespan and stability while being applied to mass-produced commercial goods. Therefore, the development of an organic optoelectronic compound that not only covers near-infrared light absorption but also exhibits excellent thermal stability in device performance is a critical issue at present.

### SUMMARY OF THE INVENTION

In view of this, one category of the present invention is to provide an organic optoelectronic compound comprises a structure such as Formula I:

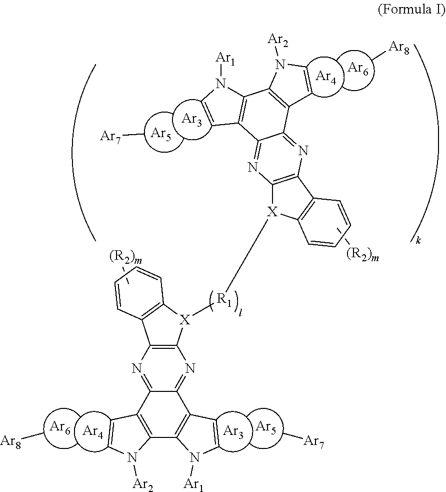

Wherein, R₁ is selected from the following groups and their derivatives consisting of C1˜C30 alkyl, C6˜C30 aryl, C3˜C30 heteroaryl, C1˜C8 haloalkyl, C4˜C30 aralkyl, C4˜C30 alkoxy and polymer chain. R₂ and R₁ can be the same or different, R₂ is selected from the following groups and their derivatives consisting of C1˜C30 alkyl, C6˜C30 aryl, C3˜C30 heteroaryl, C1˜C8 haloalkyl, C4˜C30 aralkyl, C4˜C30 alkoxy, hydrogen, fluorine, chlorine and bromine. Ar₁ and Ar₂ can be the same or different, and are independently selected from the following groups and their derivatives consisting of C6˜C30 carbon chain, C6˜C30 cycloalkyl, C6˜C30 aryl, C2˜C30 heteroaryl, C1˜C30 alkoxy and C7˜C30 phenalkyl. Ar₃ to Ar₆ can be the same or different, and are independently selected from the following groups and their derivatives consisting of C6˜C30 aryl, C2˜C30 heteroaryl and C2˜C8 spiro. Ar₇ and Ar₈ can be the same or different electron-withdrawing groups, and each of the electron-withdrawing groups is a polycyclic structure, with or without substituents, comprising at least one five-membered ring and at least one six-membered ring; a polycyclic structure, with or without substituents, comprising at least two five-membered rings; or a polycyclic structure, with or without substituents, comprising at least two six-membered rings. X is selected from the following group consisting of nitrogen, oxygen, sulfur and selenium. Wherein, when X is nitrogen, k≥0, 1 is an integer from 1 to 10, and m is an integer from 0 to 10; and when X is oxygen, sulfur, or selenium, k and 1 are 0, and m is an integer from 0 to 10.

Wherein, when X is nitrogen and R₁ is a non-polymer chain, k is an integer from 0 to 2, and 1 is an integer from 1 to 10, and m is an integer between 0 and 10.

Wherein, when X is nitrogen and R₁ is a polymer chain, k≥0, 1 is 1, and m is an integer from 0 to 10.

Wherein, Ar₁ and Ar₂ are independently selected from the following structures:

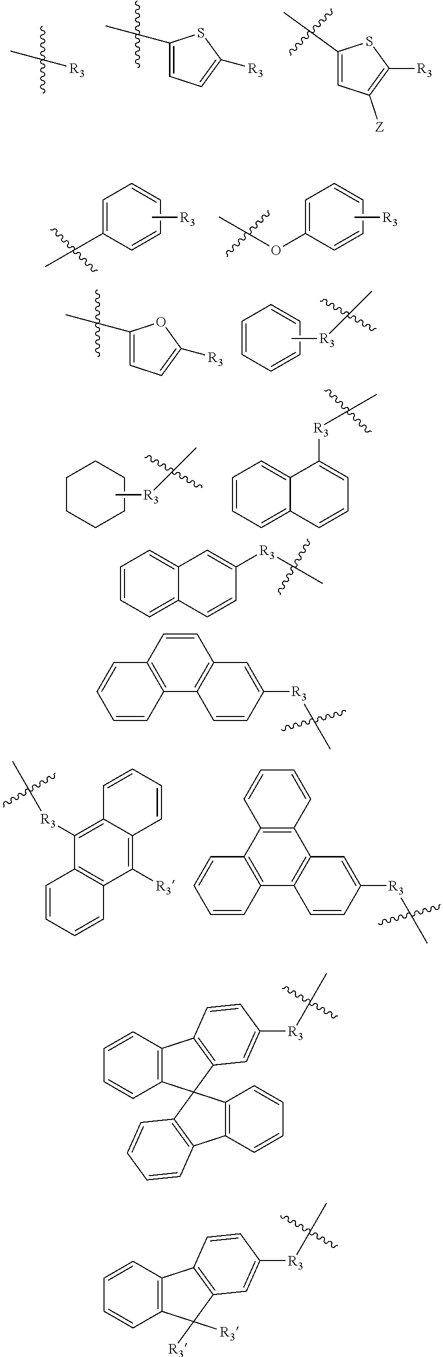

Wherein, Z comprises halogen. R₃ is selected from the following groups and their derivatives consisting of C1˜C30 linear alkyl, C1˜C30 branched alkyl and C1˜C30 alkoxy. R₃′ is selected from the following groups and their derivatives consisting of C1˜C30 linear alkyl, C1˜C30 branched alkyl, C6˜C30 aryl, C2˜C30 heterocycle, C1˜C30 alkoxy and C7˜C30 phenalkyl.

Wherein, Ar₃ and Ar₄ are independently selected from the following structures:

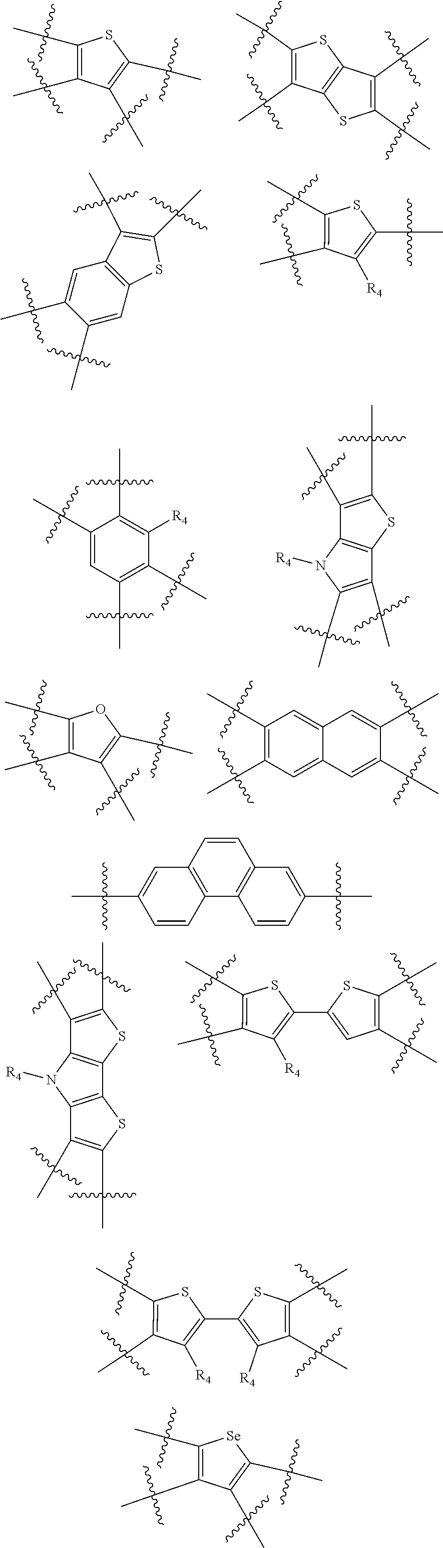

Wherein, R₄ is selected from the following groups and their derivatives consisting of C1˜C30 alkyl, C2˜C30 heterocycle, C1˜C30 alkoxy and C7˜C30 phenalkyl.

Wherein, Ar₅ and Ar₆ are independently selected from the following structures:

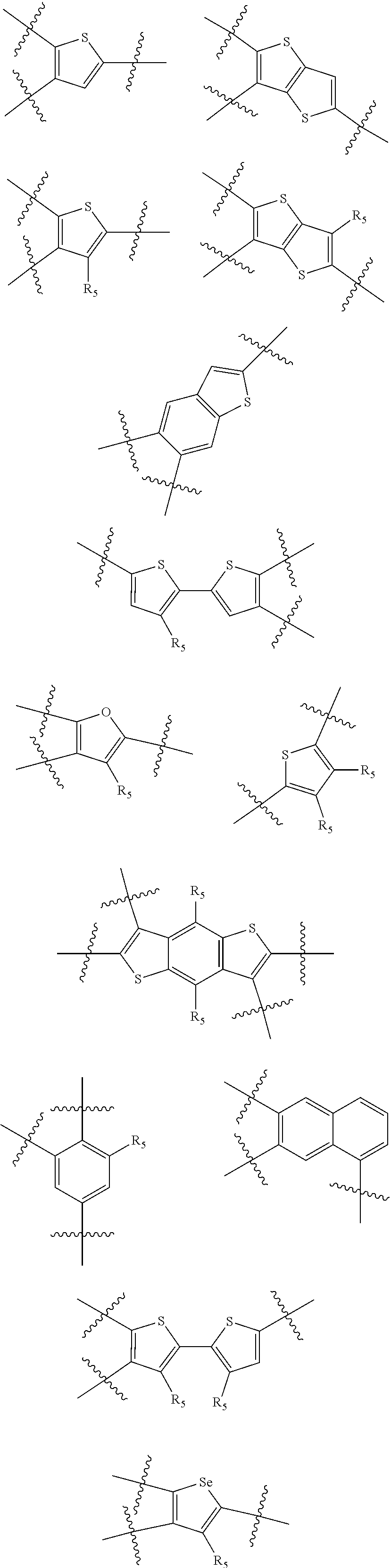

Wherein, R₅ is selected from the following groups and their derivatives consisting of C1˜C30 alkyl, C6˜C30 aryl, C2˜C30 heterocycle, C1˜C30 alkoxy, C7˜C30 phenalkyl, hydrogen, fluorine and chlorine.

Wherein, the polycyclic structure of Ar₇ and Ar₈ comprises at least one of the following two: at least one of the five-membered rings comprises at least one of cyano and C═O, and at least one of the six-membered rings comprises at least one of hydrogen, halogen and C═O as a substituent.

Second category of the present invention is to provide an active layer material comprising an acceptor material and a donor material. The acceptor material comprises an organic optoelectronic compound aforementioned. The donor material comprises at least one organic conjugated polymer.

Wherein, the organic conjugated polymer is selected from the following structures:

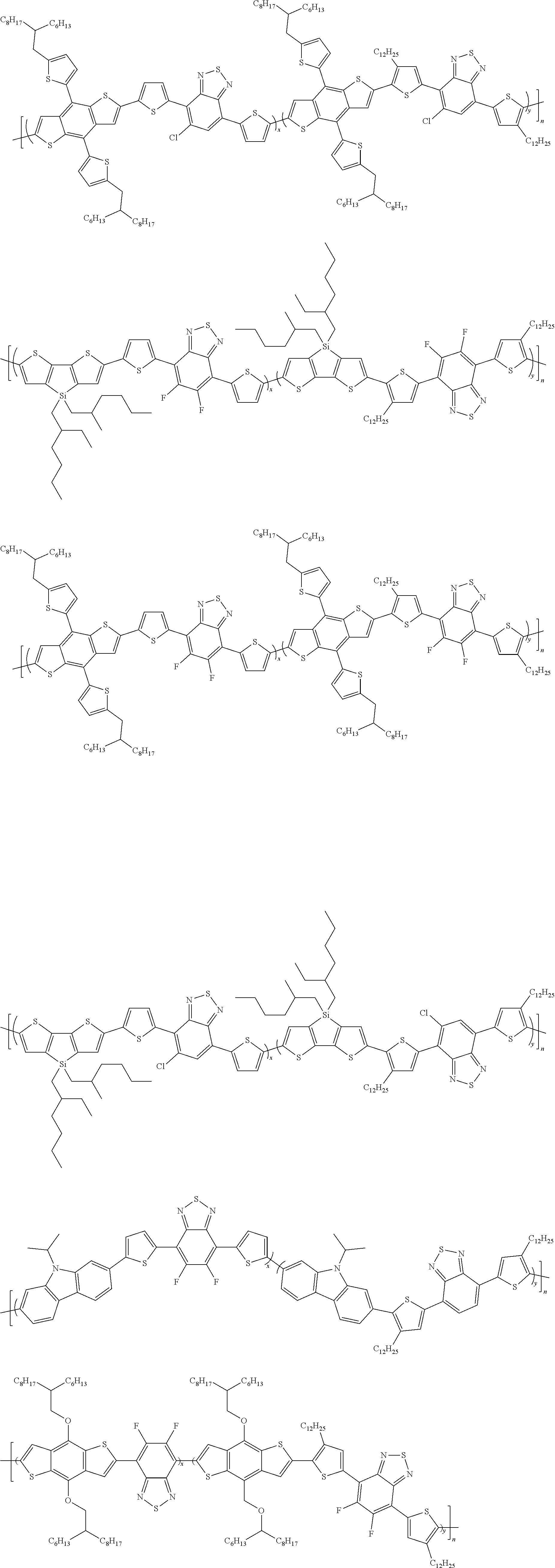

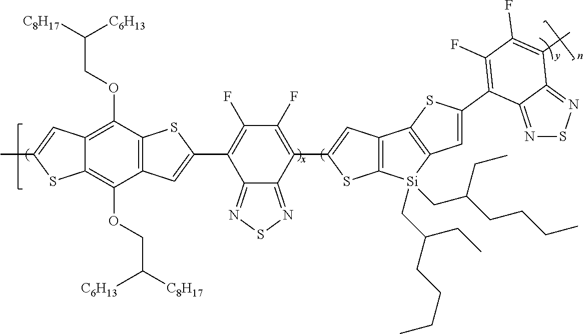

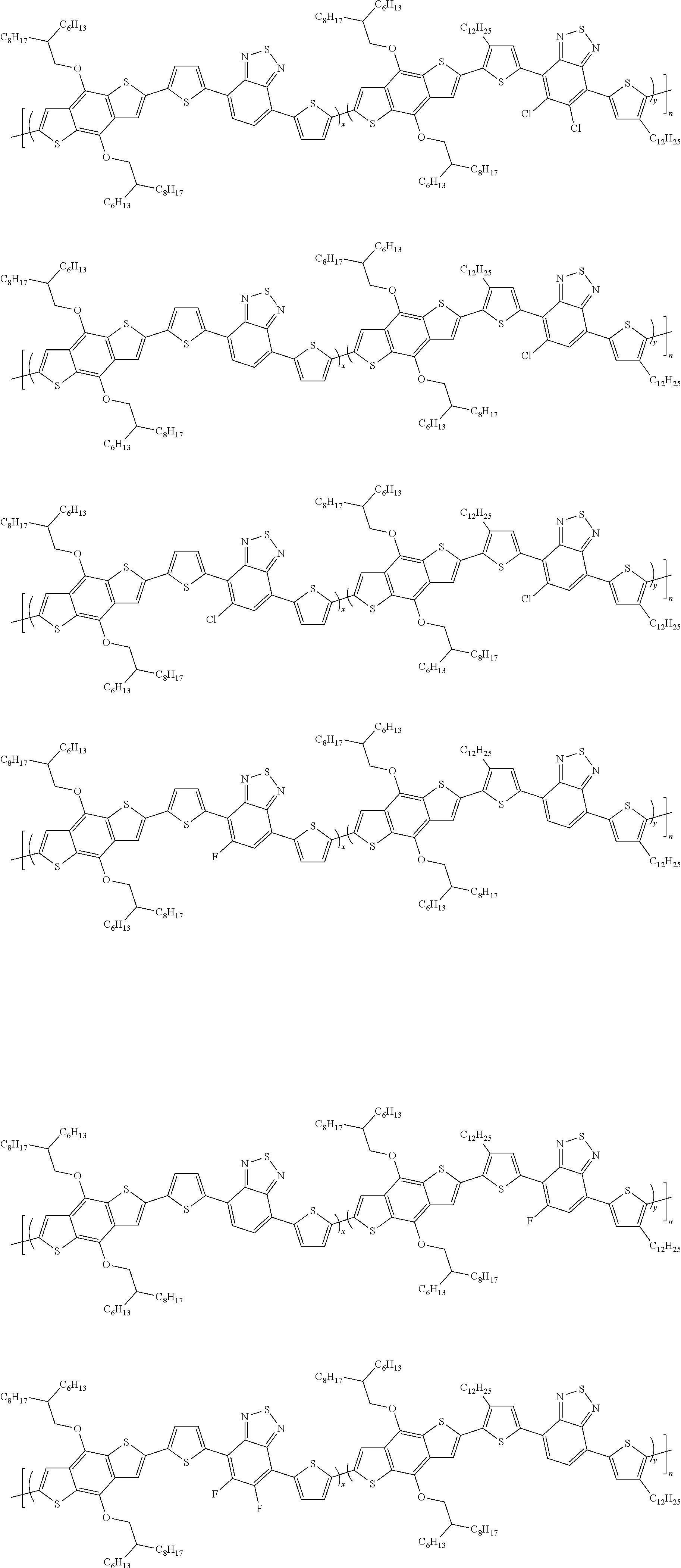

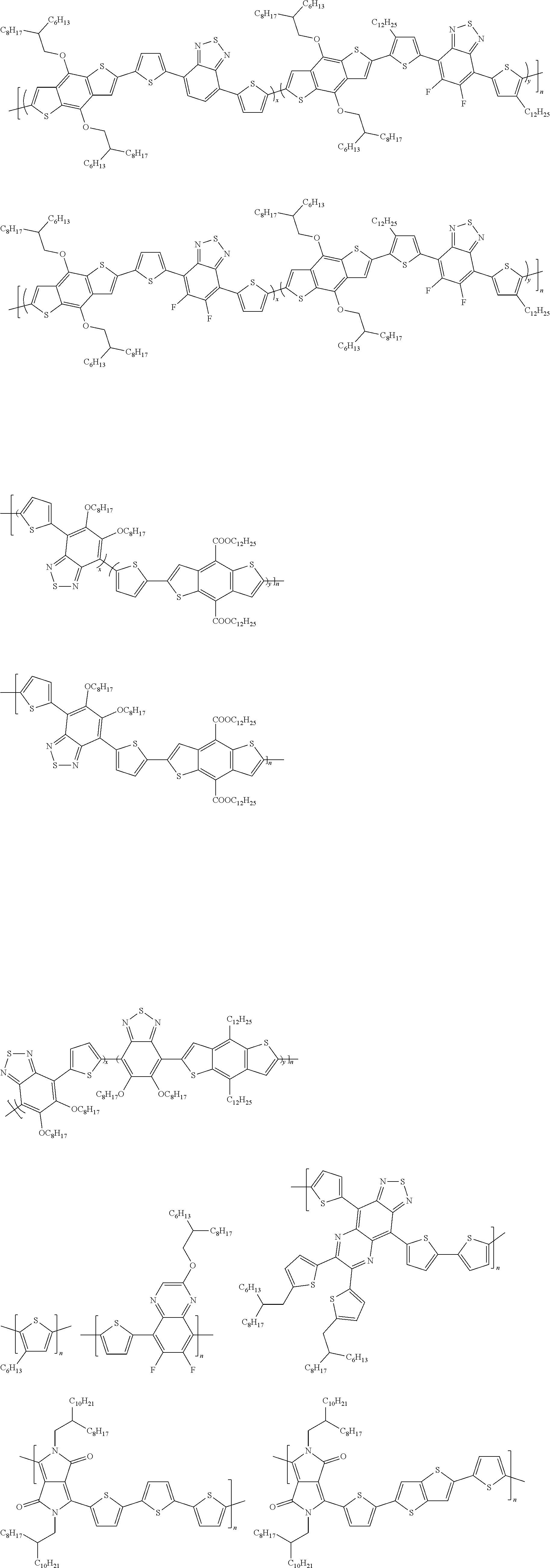

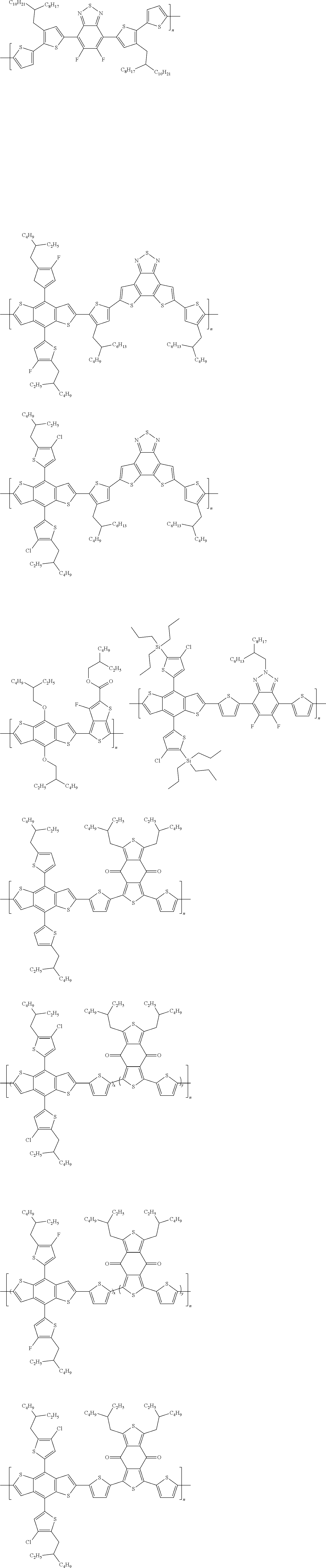

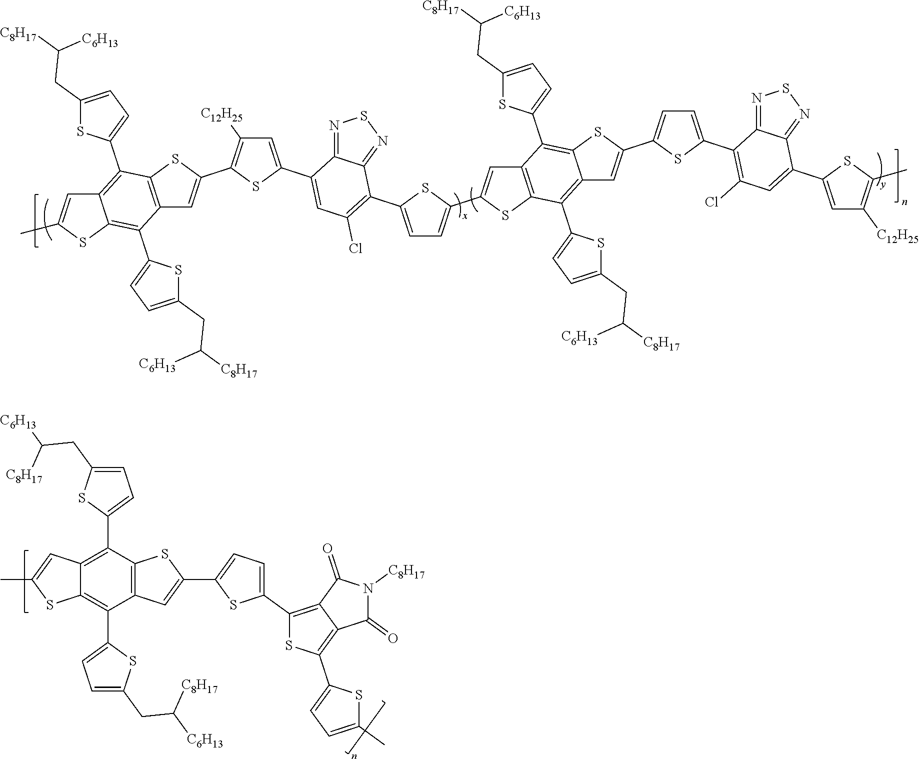

Wherein, 0<x<1, 0<y<1, and x+y=1; and n is a positive integer greater than 1.

Third category of the present invention is to provide an organic optoelectronic device comprising a first electrode, an active layer and a second electrode. The active layer comprises at least one of an organic optoelectronic compound aforementioned and an active layer material aforementioned. Wherein, the active layer is disposed between the first electrode and the second electrode, and at least one of the first electrode and the second electrode is a transparent or semi-transparent electrode.

Wherein, the organic optoelectronic device further comprises a first carrier transporting layer and a second carrier transporting layer. The first carrier transporting layer is disposed between the first electrode and the active layer and the first carrier transporting layer is configured to transport carriers in the first electrode and the active layer. The second carrier transporting layer is disposed between the active layer and the second electrode and the second carrier transporting layer is configured to transport carriers in the active layer and the second electrode.

Compared with the prior art, the organic optoelectronic device of the present invention has good thermal stability.

### BRIEF DESCRIPTION OF THE APPENDED DRAWINGS

Some of the embodiments will be described in detail, with reference to the following figures, wherein like designations denote like members, wherein:

FIG. 1 shows a schematic structural diagram of one embodiment of an organic optoelectronic device of the present invention.

FIG. 2 shows thermal stability test results of film surfaces of a Comparative Example Ref., and embodiments of N01 and N15 of the present invention.

FIG. 3 shows thermal stability test results of film surfaces of embodiments of D01 and P01 of the present invention.

### DETAILED DESCRIPTION OF THE INVENTION

In order to make the advantages, spirit and features of the present invention easier and clearer, it will be detailed and discussed in the following with reference to the embodiments and the accompanying drawings. It is worth noting that the specific embodiments are merely representatives of the embodiments of the present invention, but it can be implemented in many different forms and is not limited to the embodiments described in this specification. Rather, these embodiments are provided so that this disclosure will be thorough and complete.

The terminology used in the various embodiments disclosed in the present invention is only for the purpose of describing specific embodiments, and is not intended to limit the various embodiments disclosed in the present invention. As used herein, singular forms also include plural forms unless the context clearly indicates otherwise. Unless otherwise defined, all terms (including technical and scientific terms) used in this specification have the same meanings as commonly understood by one of ordinary skill in the art to which the various embodiments disclosed herein belong. The above terms (such as those defined in commonly used dictionaries) will be interpreted as having the same meaning as the contextual meaning in the same technical field, and will not be interpreted as having an idealized or overly formal meaning, unless explicitly defined in the various embodiments disclosed herein.

In the description of this specification, the description of the reference terms “an embodiment”, “a specific embodiment” and the like means that specific features, structures, materials, or characteristics described in connection with the embodiment are included in at least one embodiment of the present invention. In this specification, the schematic expressions of the above terms do not necessarily refer to the same embodiment. Furthermore, the particular features, structures, materials, or characteristics described may be combined in any suitable manner in any one or more embodiments.

### Definition

As used herein, “donor” material and “p-type” (“P-type”) material refer to a semiconductor material, such as an organic semiconductor material, having holes as a primary current or charge carrier. In some embodiments, when a p-type semiconductor material is deposited on a substrate, it can provide the hole mobility greater than about 10⁻⁵ cm²/Vs.

As used herein, “acceptor” material and “n-type” (“N-type”) material refer to the semiconductor material, such as the organic semiconductor material, having electrons as the primary current or the charge carrier. In some embodiments, when an n-type semiconductor material is deposited on a substrate, it can provide the electron mobility of more than about 10⁻⁵ cm²/Vs.

The “electron-withdrawing group” refers to a group or an atom with a stronger electron-withdrawing ability than that of hydrogen, that is, it has an electron-withdrawing inductive effect. The “electron-donating group” refers to a group or an atom whose electron-donating ability is stronger than that of hydrogen, that is, it has an electron-donating induction effect. The inductive effect is the effect that the bonding electron cloud moves in a certain direction on the atomic bond due to the difference in polarity (electronegativity) of atoms or groups in the molecule. The electron cloud tends to move towards the groups or atoms with more electronegativity.

“ [symbol] ”, “ [symbol] ” or “*” in the structures listed herein represents the available bonding positions of this structure, but not limited thereto.

As used herein, “polymer” refers to a very large molecule consisting of thousands of covalently bonded atoms. Polymer is composed of many repeating units, that is, monomers (composed of one or more atoms) bonded together by covalent bonds. From the point of view of physical properties, the number of repeating units should be so large that adding some more repeating units will not significantly change the physical properties. Polymer is further divided into copolymer and homopolymer. The homopolymer is polymerized from only one type of monomer. The copolymer is polymer formed by the polymerization of two or more monomers. The copolymer is divided into alternating copolymer, random copolymer, block copolymer and graft copolymer.

As used herein, “component” (such as a thin film layer) may be considered “photoactive” if it contains one or more compounds that absorb photons to generate excitons for generating photocurrent.

As used herein, “solution proceeding” refers to a process in which a compound (e.g., a polymer), material, or composition can be used in a solution state, such as spin coating, printing (e.g., inkjet printing, gravure printing, and lithography printing), spray coating, slit coating, drop casting, dip coating, and blade coating.

As used herein, “annealing” refers to a post-deposition thermal treatment to a semi-crystalline polymer film for certain duration in the environment or under decompressed or pressurized environment. “Annealing temperature” refers to the temperature at which the polymer film or the mixed film of the polymer and other molecules can perform small-scale molecular movement and rearrangement during the annealing process. Without being limited by any particular theory, it is believed that annealing can lead to an increase in crystallinity in the polymer film and enhance the carrier mobility of the polymer film or a mixed film formed by the polymer and other molecules, and the molecules are arranged alternately to achieve the effect of independent transporting paths of effective electrons and holes.

The external quantum efficiency (EQE) as used herein is the spectral response Amp/Watt unit, which Amp is converted to the number of electrons per unit time (electron/sec) and Watt is converted to the number of photons per unit time (Photons/sec), and insert the quantum efficiency obtained by the above formula. Generally speaking, quantum efficiency (QE) refers to external quantum efficiency (EQE), also known as incident photon-electron conversion efficiency (IPCE).

Due to the existing technology, ladder-shaped molecules such as Y6 (BTP-4F) formed by the A-D-A′-D-A structure have insufficient thermal stability. In view of this, the present invention redesigns the structural core on the premise of increasing the thermal stability of the non-fullerene acceptor of the A-D-A′-D-A structure type and maintaining the high efficiency characteristics of this material. This is intended to expand both the breadth and depth of its application in relevant fields once commercialized.

In an embodiment, an organic optoelectronic compound comprises a structure such as Formula I:

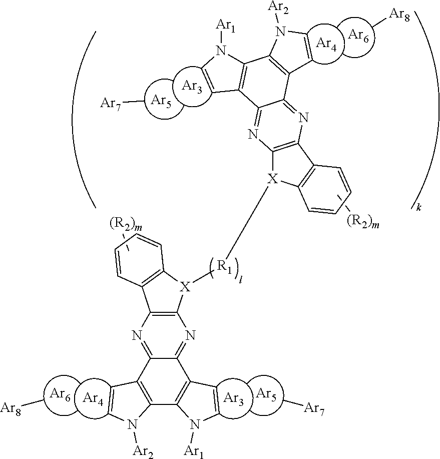

Wherein, R₁ is selected from the following groups and their derivatives consisting of C1˜C30 alkyl with or without substituents, C6˜C30 aryl with or without substituents, C3˜C30 heteroaryl with or without substituents, C1˜C8 haloalkyl with or without substituents, C4˜C30 aralkyl with or without substituents, C4˜C30 alkoxy with or without substituents and polymer chain with or without substituents. R₂ and R₁ can be the same or different, R₂ is selected from the following groups and their derivatives consisting of C1˜C30 alkyl with or without substituents, C6˜C30 aryl with or without substituents, C3˜C30 heteroaryl with or without substituents, C1˜C8 haloalkyl with or without substituents, C4˜C30 aralkyl with or without substituents, C4˜C30 alkoxy with or without substituents, hydrogen, fluorine, chlorine and bromine. Ar₁ and Ar₂ can be the same or different, and are independently selected from the following groups and their derivatives consisting of C6˜C30 carbon chain with or without substituents, C6˜C30 cycloalkyl with or without substituents, C6˜C30 aryl with or without substituents, C2˜C30 heteroaryl with or without substituents, C1˜C30 alkoxy with or without substituents and C7˜C30 phenalkyl with or without substituents. Ar₃ to Ar₆ can be the same or different, and are independently selected from the following groups and their derivatives consisting of C6˜C30 aryl with or without substituents, C2˜C30 heteroaryl with or without substituents and C2˜C8 spiro with or without substituents. Ar₇ and Ar₈ can be the same or different electron-withdrawing groups, and each of the electron-withdrawing groups is a polycyclic structure, with or without substituents, comprising at least one five-membered ring and at least one six-membered ring; a polycyclic structure, with or without substituents, comprising at least two five-membered rings; or a polycyclic structure, with or without substituents, comprising at least two six-membered rings. X is selected from the following group consisting of nitrogen, oxygen, sulfur and selenium. Wherein, when X is nitrogen, k≥0, 1 is an integer from 1 to 10, and m is an integer from 0 to 10; and when X is oxygen, sulfur, or selenium, k and l are 0, and m is an integer from 0 to 10.

The organic optoelectronic compound of the present invention introduces pyrazine groups and additional conjugated planes, which can effectively improve the stability of the material and increase the carrier transmission channel. Wherein, R₁ and R₂ have the following characteristics: 1. By modifying R₁ and R₂, the material energy level and energy gap can be fine-tuned, which is beneficial to match different P-type materials and increase the breadth of applications; 2. R₁ and R₂ can effectively change the intermolecular force to change the material arrangement and solubility, maintaining a wide range of applications; 3. Use R₁ and R₂ as bridges between molecules to connect two or more molecules to extend the conjugated system and strengthen the network built by the intermolecular forces.

In practice, Ar₁ and Ar₂ are independently selected from the following structures:

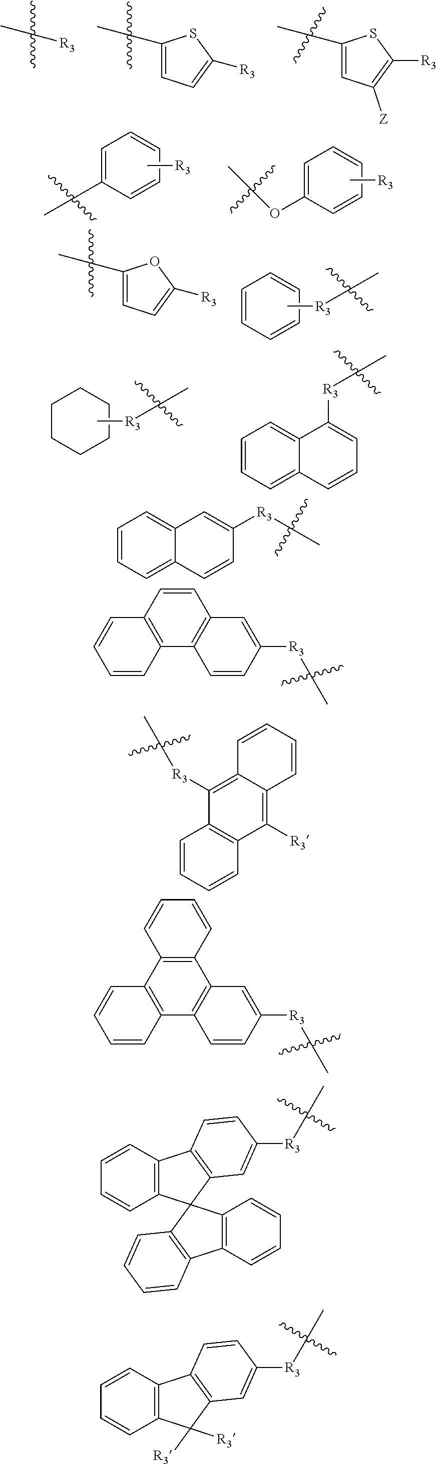

Wherein, Z comprises halogen. R₃ is selected from the following groups and their derivatives consisting of C1˜C30 linear alkyl with or without substituents, C1˜C30 branched alkyl with or without substituents and C1˜C30 alkoxy with or without substituents. R₃′ is selected from the following groups and their derivatives consisting of C1˜C30 linear alkyl with or without substituents, C1˜C30 branched alkyl with or without substituents, C6˜C30 aryl with or without substituents, C2˜C30 heterocycle with or without substituents, C1˜C30 alkoxy with or without substituents and C7˜C30 phenalkyl with or without substituents.

In practice, Ar₃ and Ar₄ are independently selected from the following

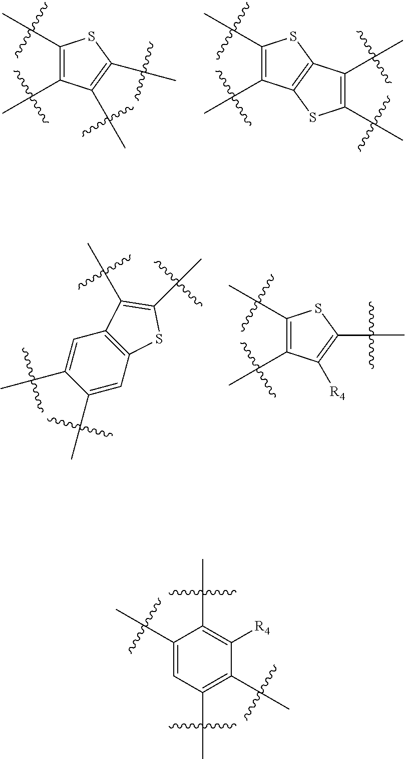

structures:

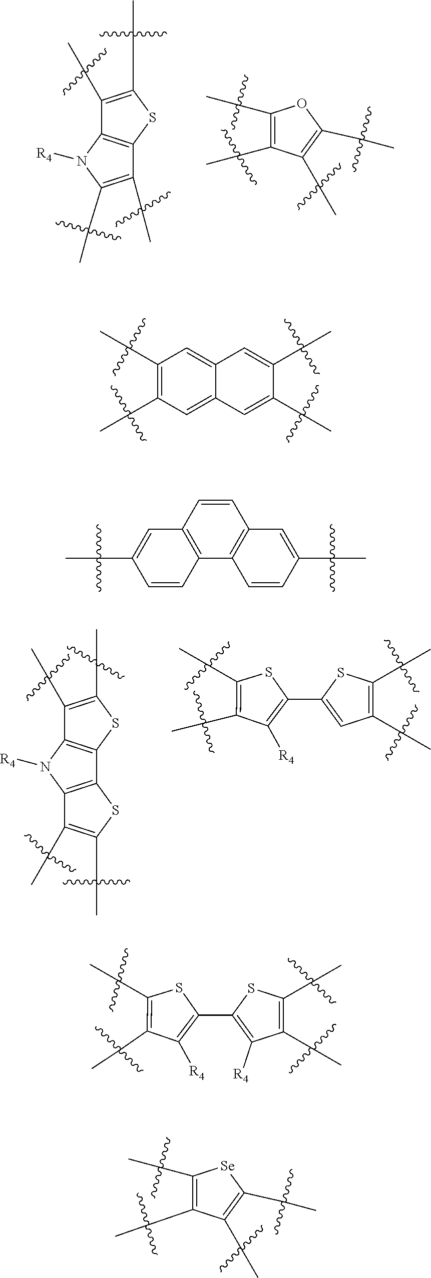

Wherein, R₄ is selected from the following groups and their derivatives consisting of C1˜C30 alkyl with or without substituents, C2˜C30 heterocycle with or without substituents, C1˜C30 alkoxy with or without substituents and C7˜C30 phenalkyl with or without substituents.

In practice, Ar₅ and Ar₆ are independently selected from the following structures:

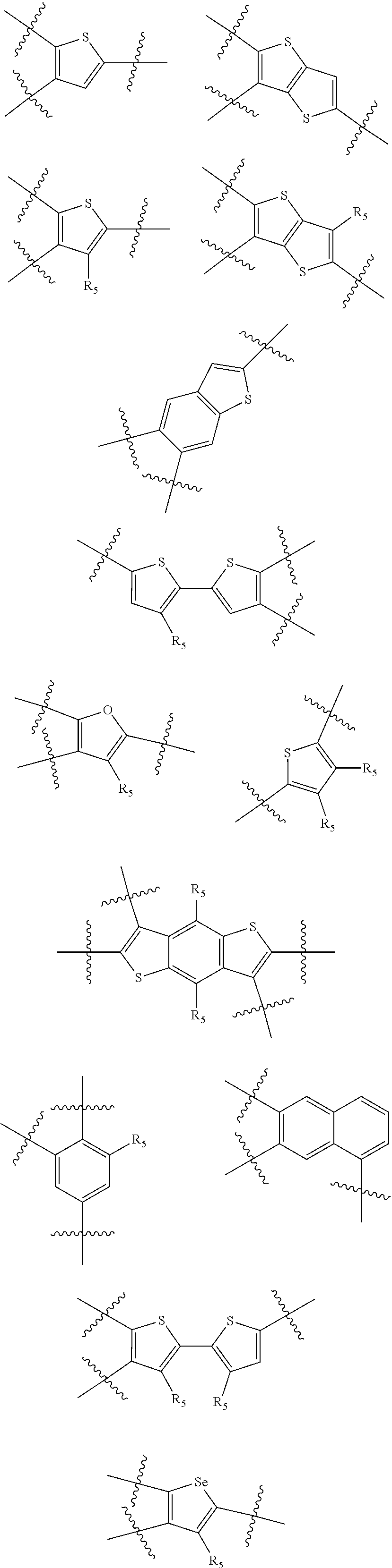

Wherein, R₅ is selected from the following groups and their derivatives consisting of C1˜C30 alkyl with or without substituents, C6˜C30 aryl with or without substituents, C2˜C30 heterocycle with or without substituents, C1˜C30 alkoxy with or without substituents, C7˜C30 phenalkyl with or without substituents, hydrogen, fluorine and chlorine.

Furthermore, the polycyclic structure of Ar₇ and Ar₈ comprises at least one of the following two: at least one of the five-membered rings comprises at least one of cyano and C═O, and at least one of the six-membered rings comprises at least one of hydrogen, halogen and C═O as a substituent.

In practice, Ar₇ and Ar₈ are independently selected from the following structures:

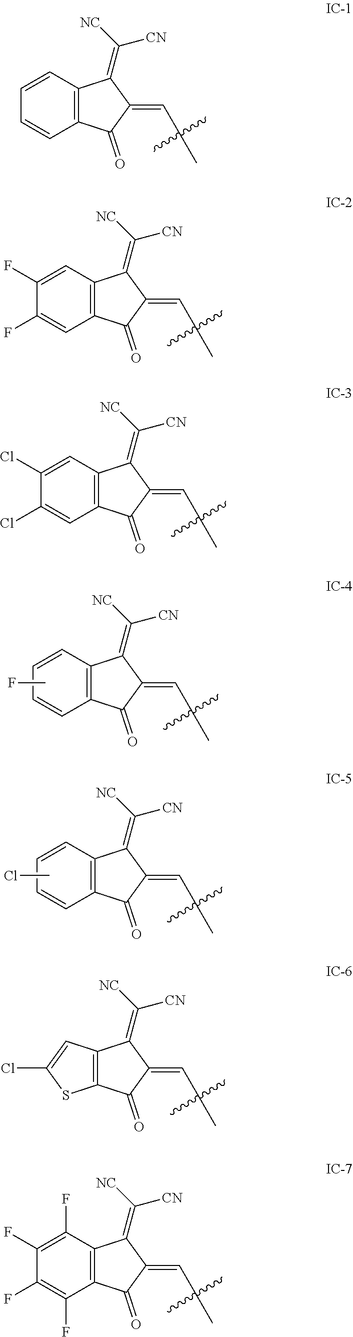

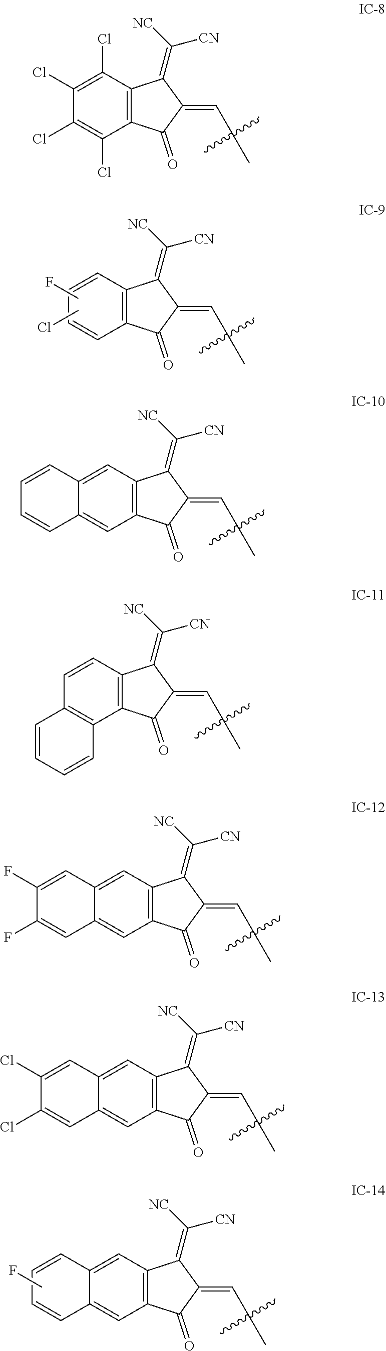

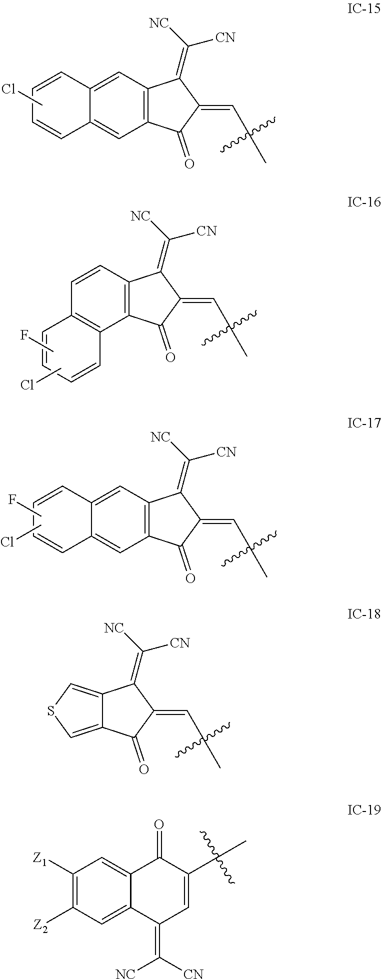

Wherein, Z₁ and Z₂ can be the same or different, and are independently selected from at least one of hydrogen atoms or halogen.

The above substituents can be independently selected from the following groups and their derivatives consisting of C1˜C30 alkyl, C3˜C30 branched alkyl, C1˜C30 silyl, C2˜C30 ester, C1˜C30 alkoxy, C1˜C30 alkylthio, C1˜C30 haloalkyl, C2˜C30 alkene, C2˜C30 alkyne, C2˜C30 carbon chain containing cyano, C1˜C30 carbon chain containing nitro, C1˜C30 carbon chain containing hydroxyl, C3˜C30 carbon chain containing ketone, halogen, cyano and hydrogen. The above-mentioned aryl and heteroaryl may contain monocyclic or polycyclic structures.

Specifically, X is selected from the following group consisting of nitrogen, oxygen, sulfur and selenium. Wherein, when X is oxygen, sulfur, or selenium, k and l are 0, and m is an integer from 0 to 10. The organic optoelectronic compound of the present invention may comprise the following examples:

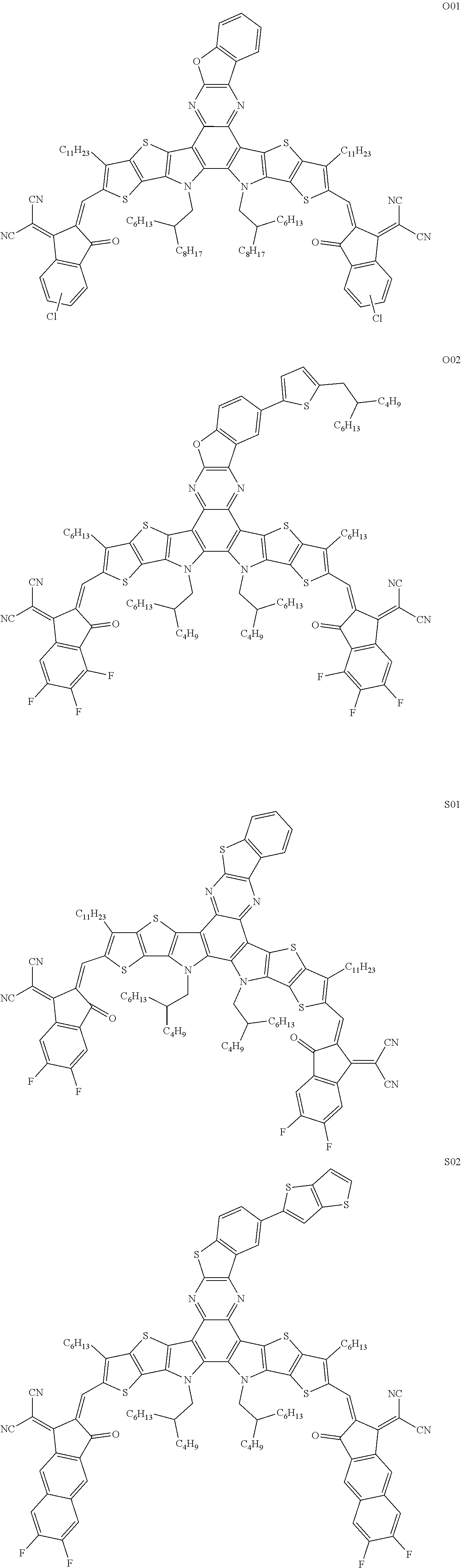

Wherein, when X is nitrogen and R₁ is a non-polymer chain, k is an integer from 0 to 2, and l is an integer from 1 to 10, and m is an integer between 0 and 10. Specifically, the organic optoelectronic compound of the present invention may comprise the following examples:

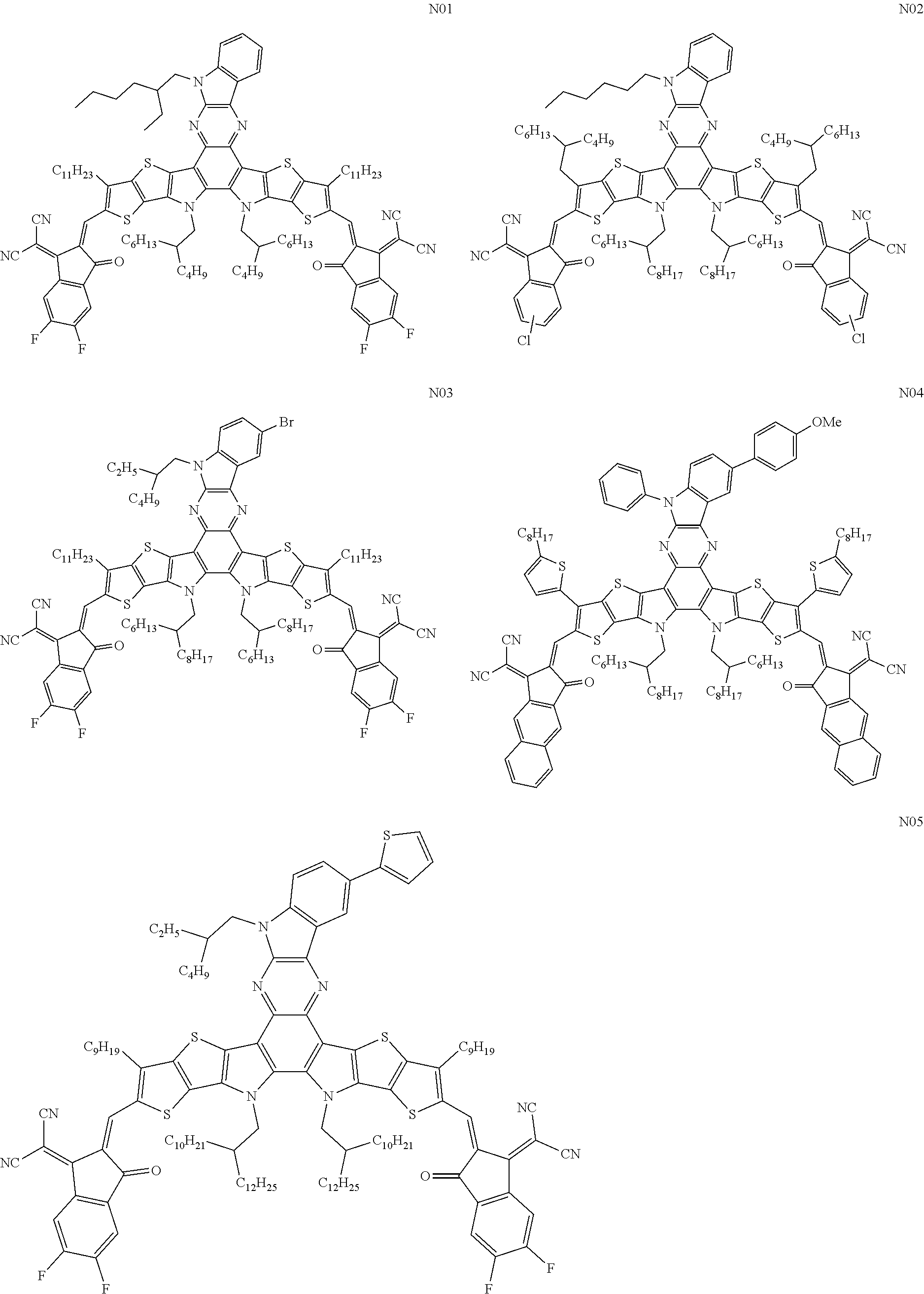

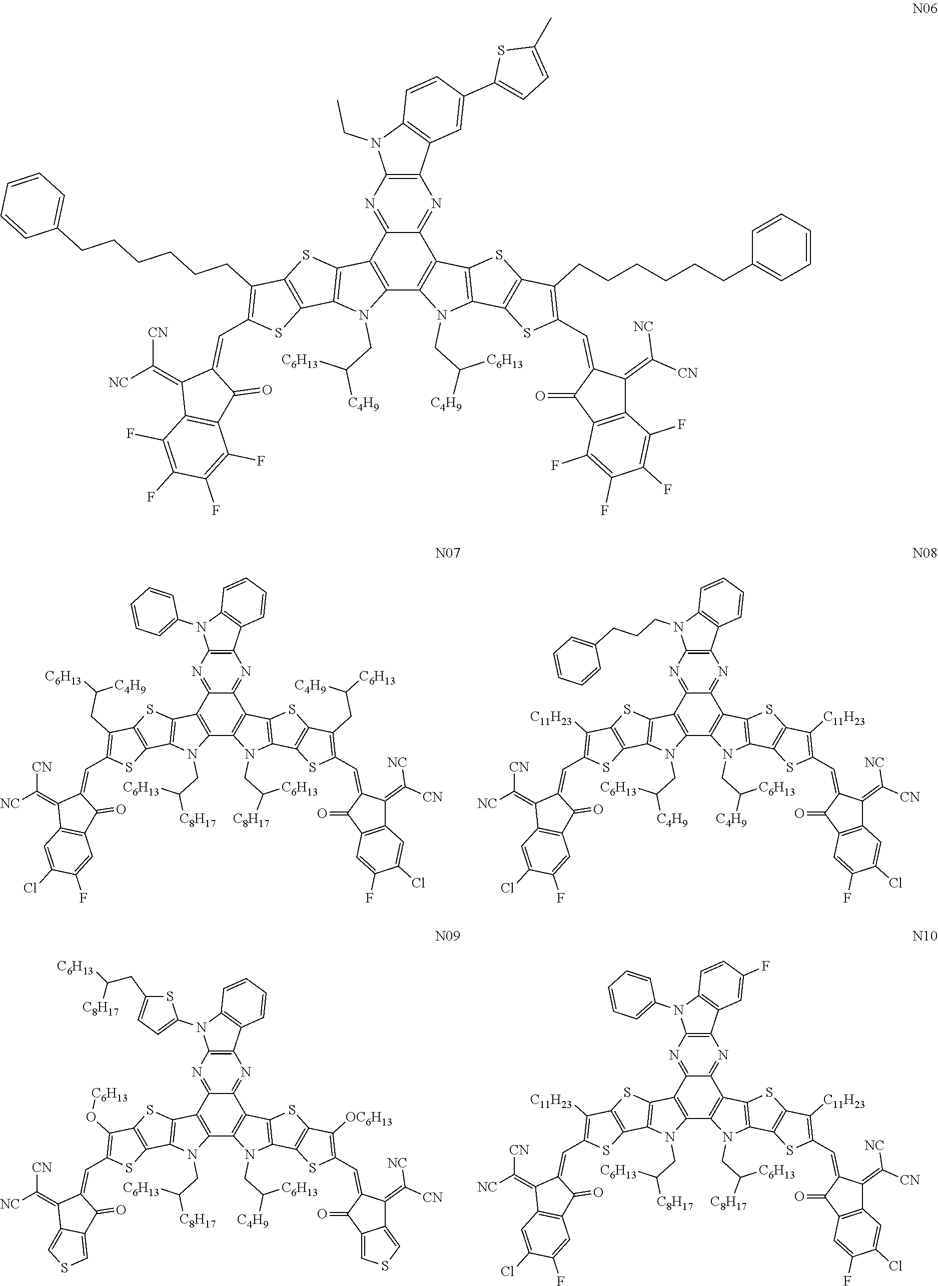

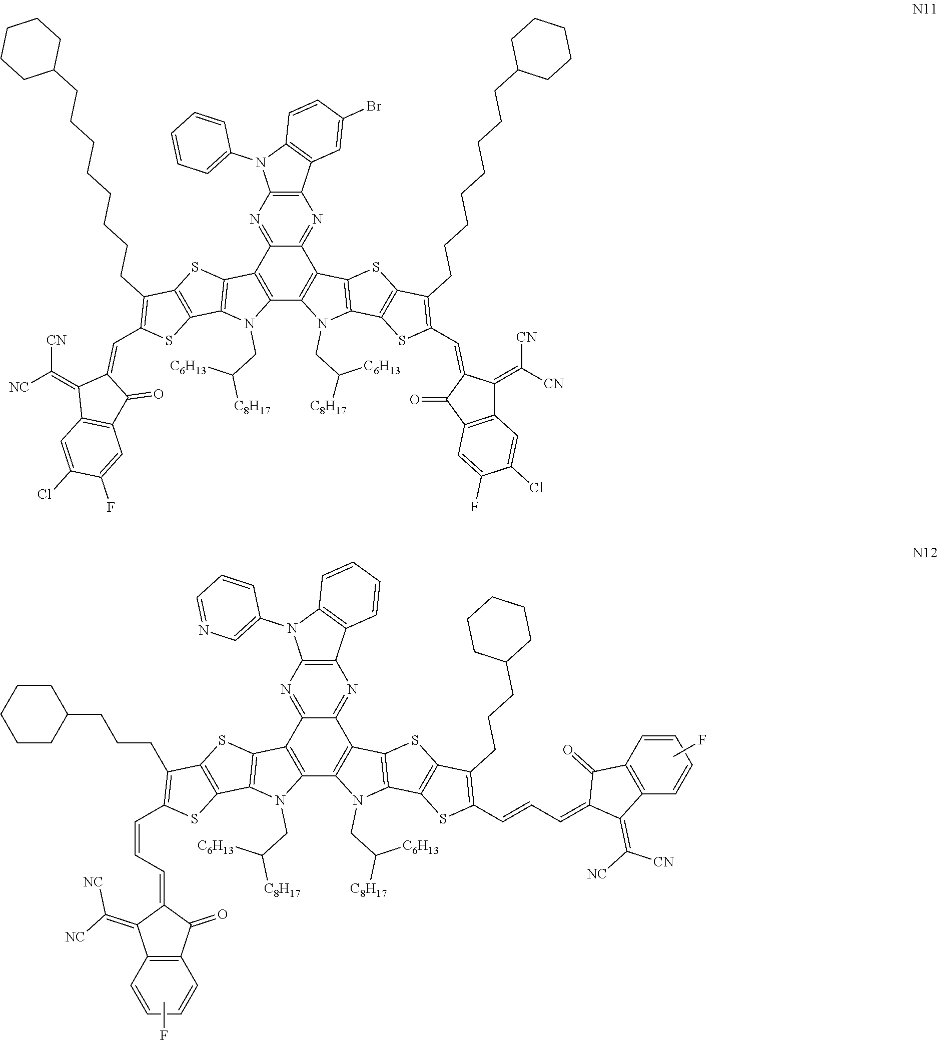

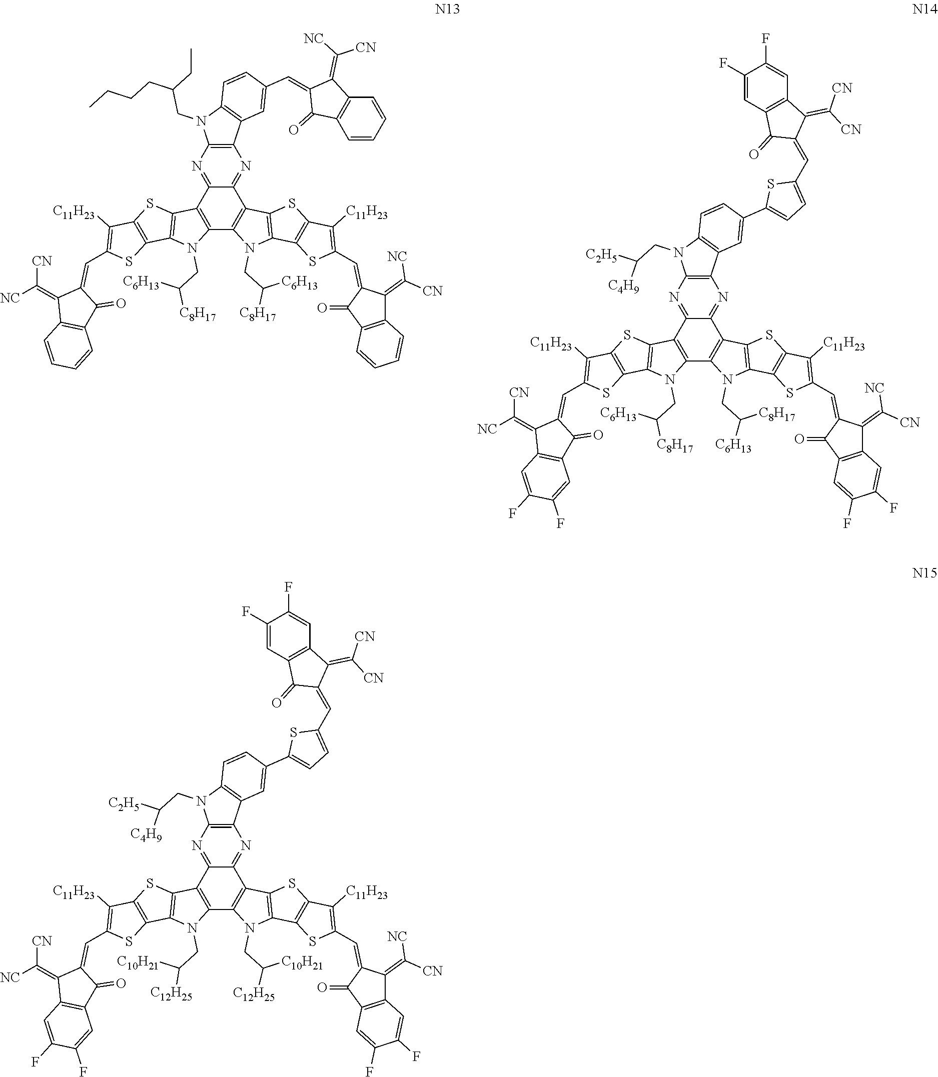

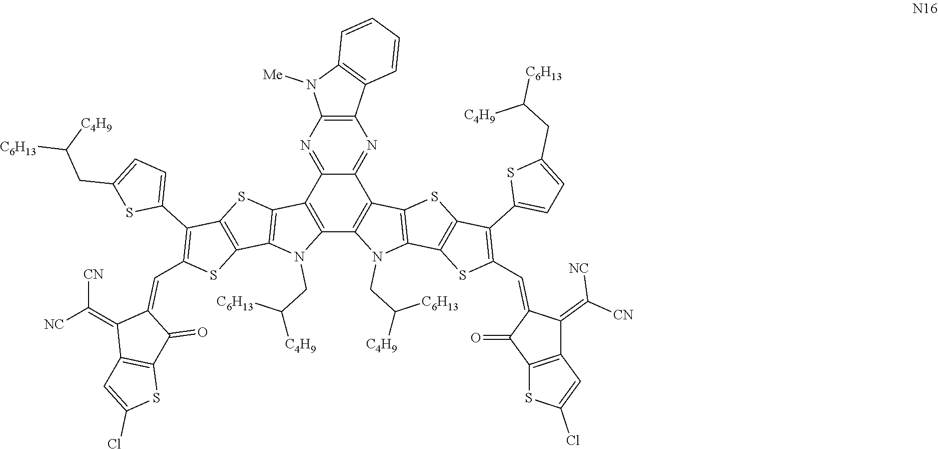

In the following, D01 to D04 will be used as examples of dimer structures, and D05 will be used as an example of trimer structure, but they are not limited to this.

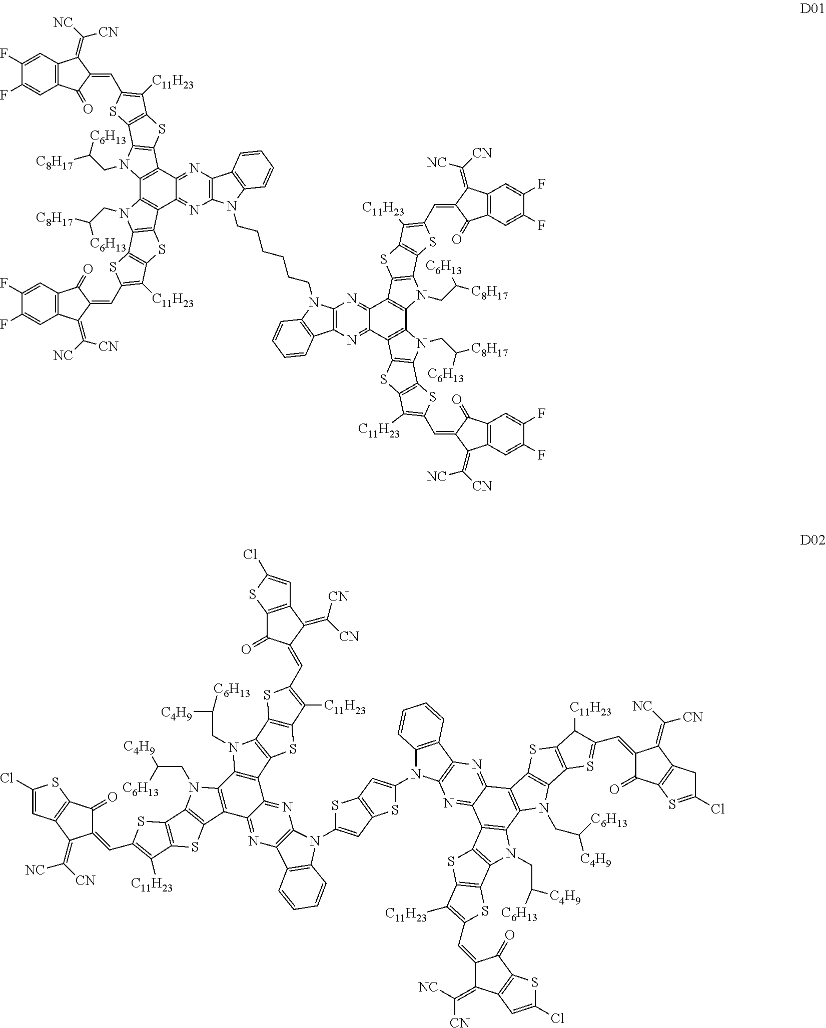

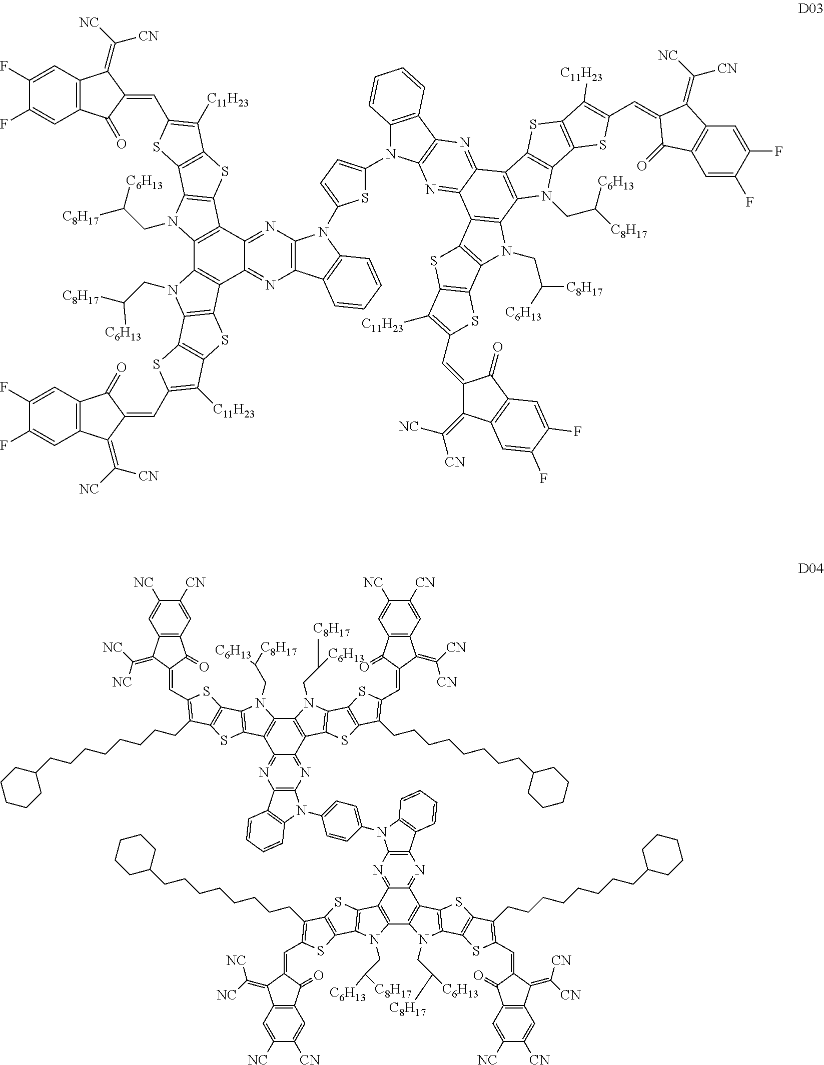

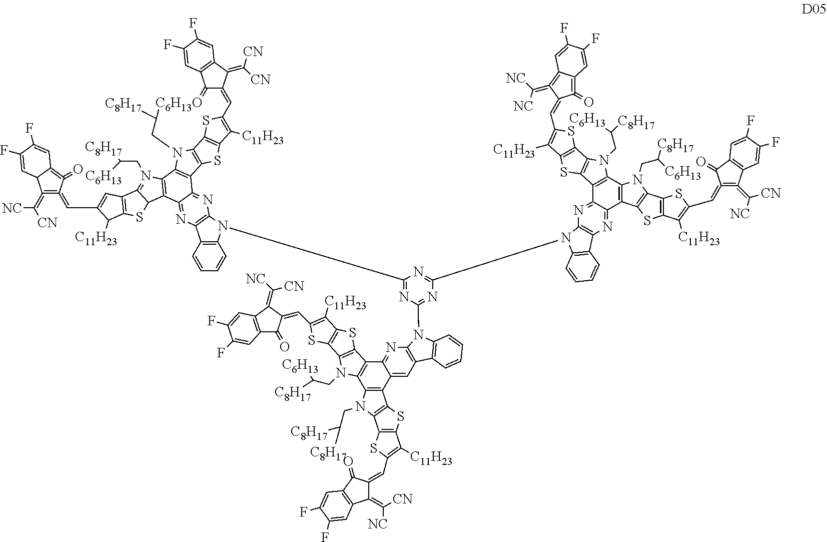

Wherein, when X is nitrogen and R₁ is a polymer chain, k≥0, l is 1, and m is an integer from 0 to 10. Specifically, the organic optoelectronic compound of the present invention may comprise the following examples:

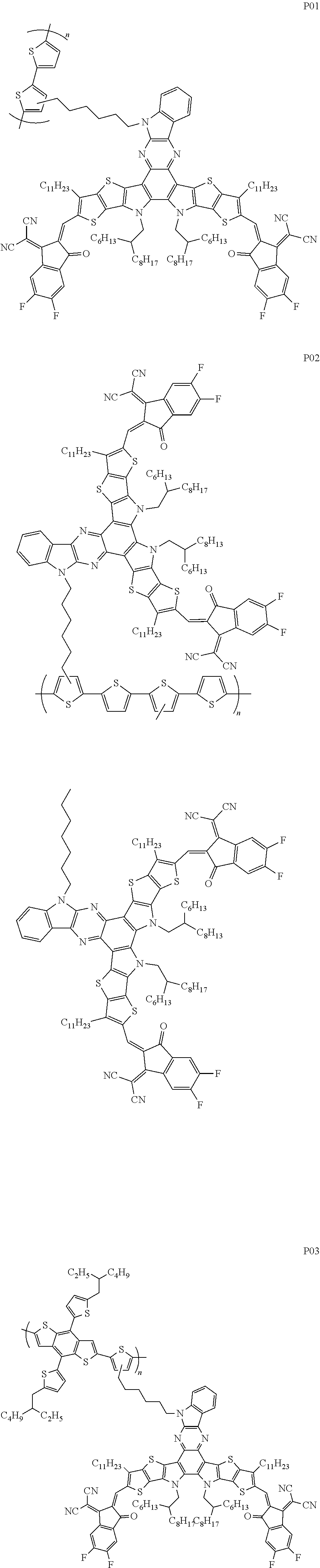

It should be understood that the above-listed embodiments are only intended to allow the person skilled in the art to understand the structure and composition of the present invention more clearly, and are not limited thereto.

In practical applications, the present invention also provides an active layer material, which comprises an acceptor material and a donor material. Wherein, acceptor material comprises an organic optoelectronic compound aforementioned. The donor material comprises at least one organic conjugated polymer.

In detail, the organic conjugated polymer is selected from the following examples:

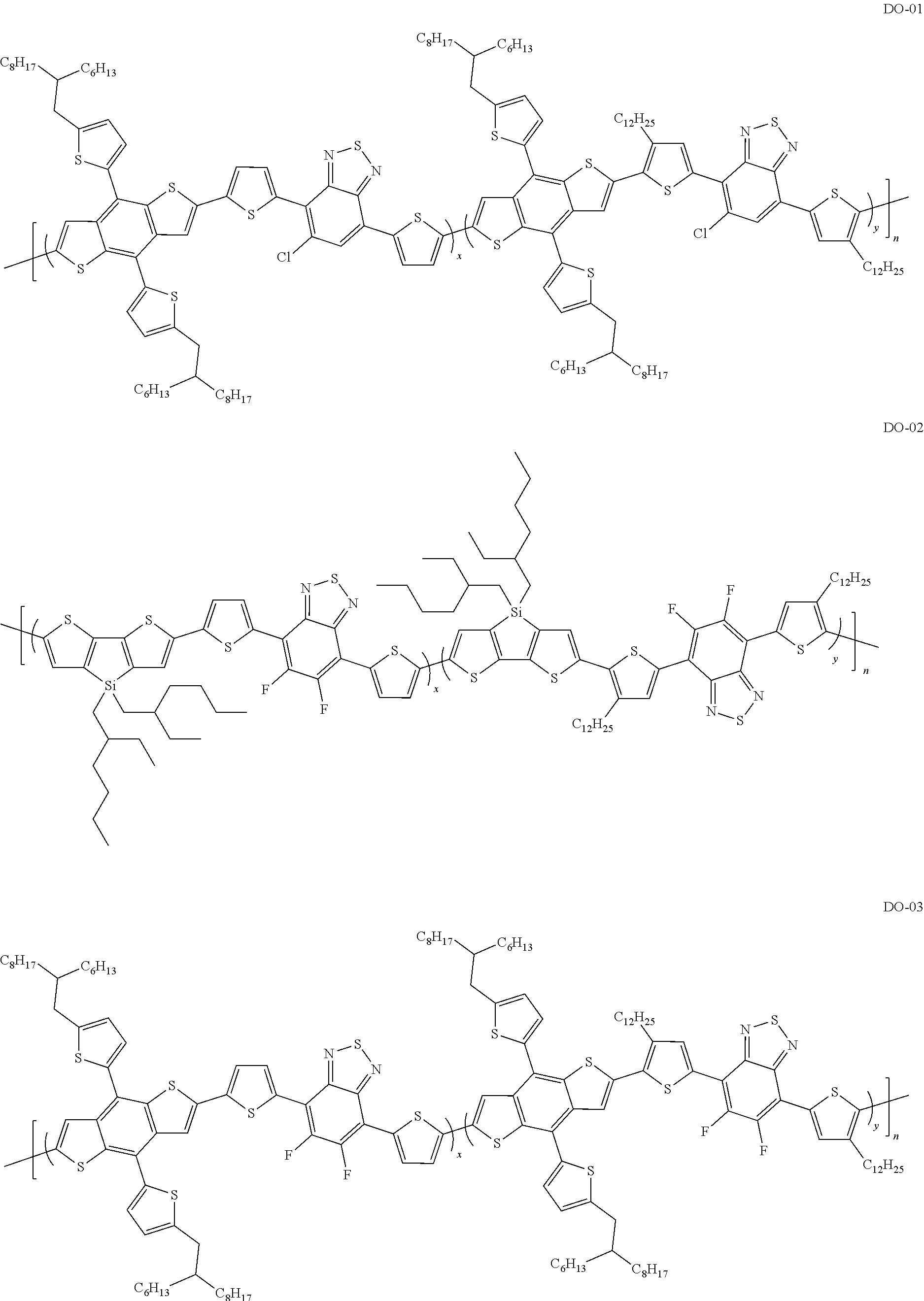

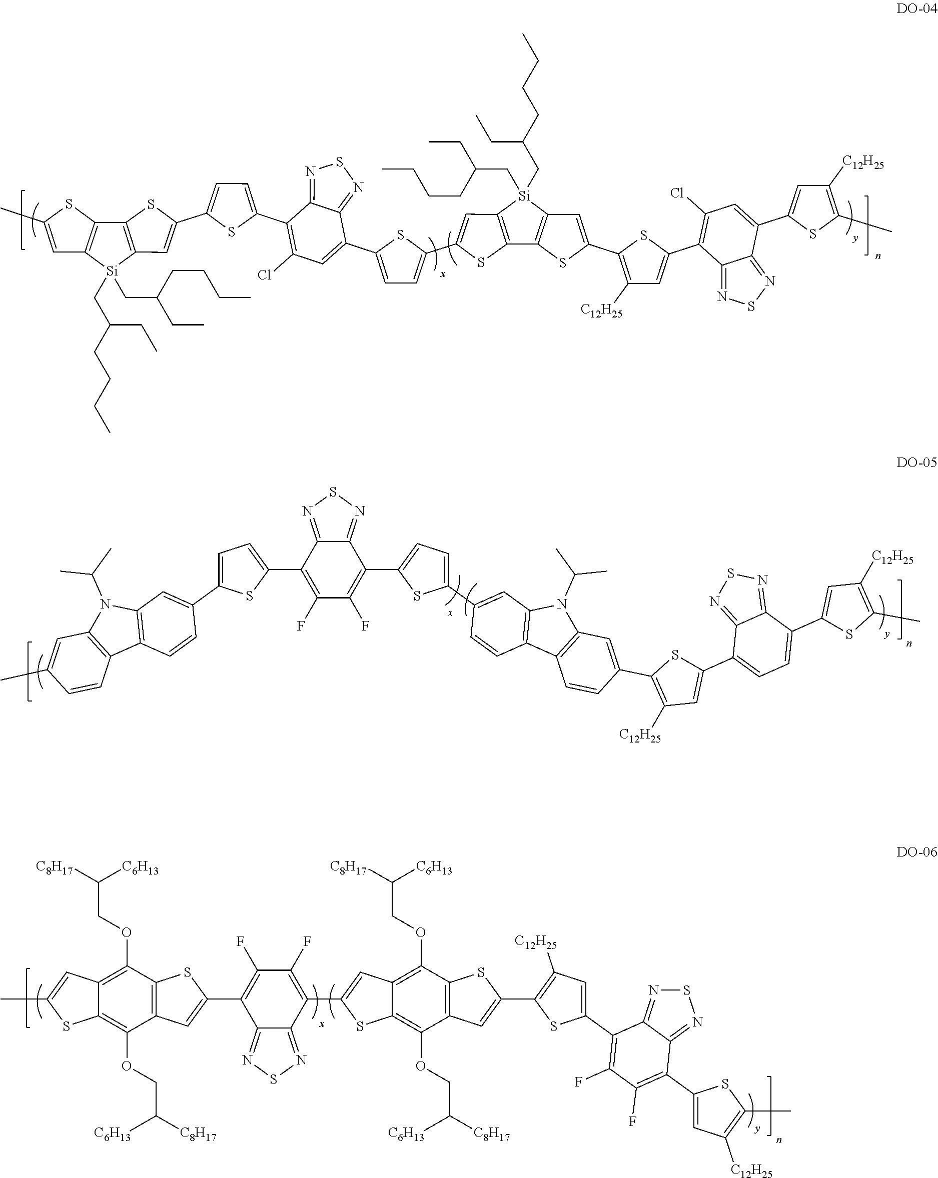

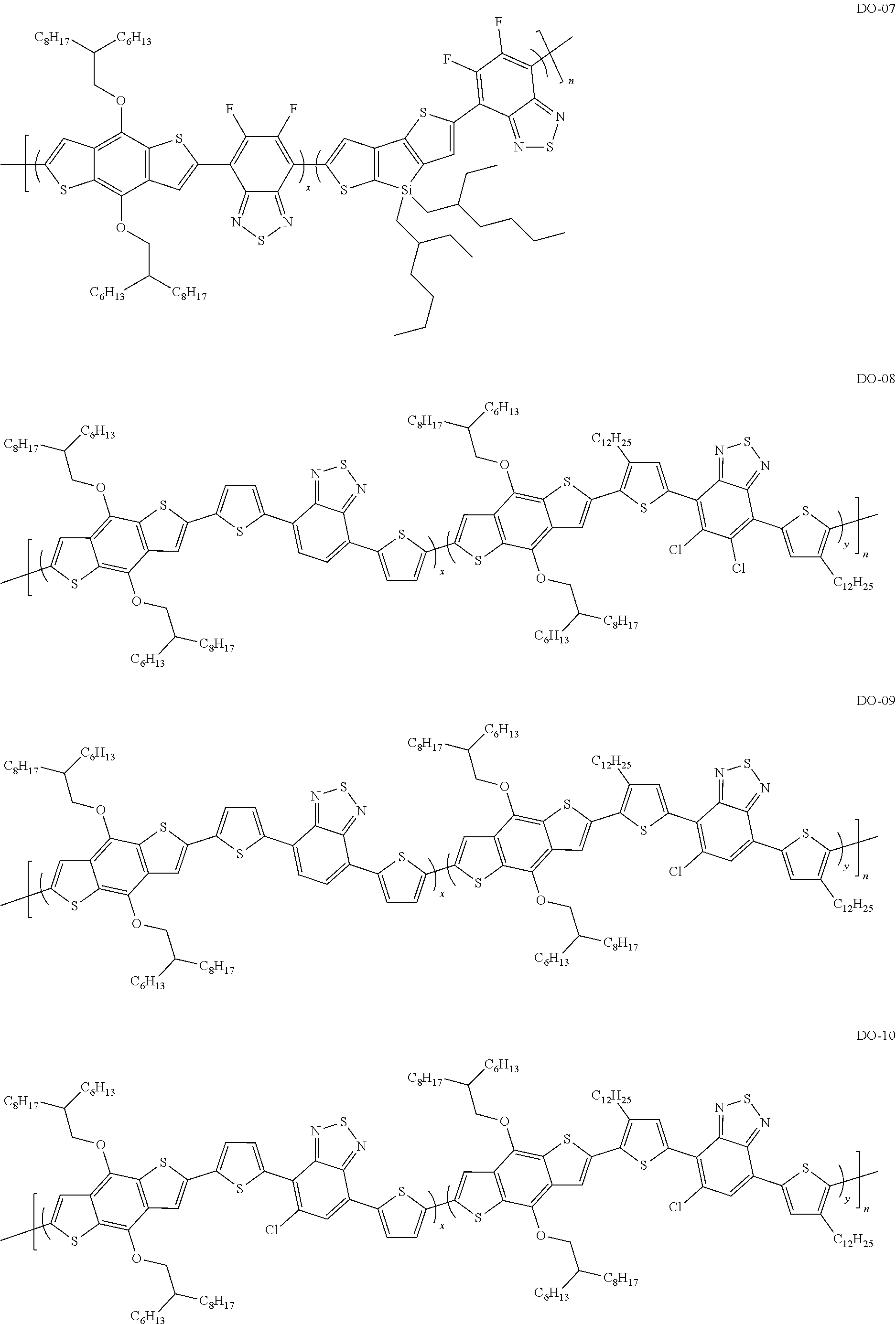

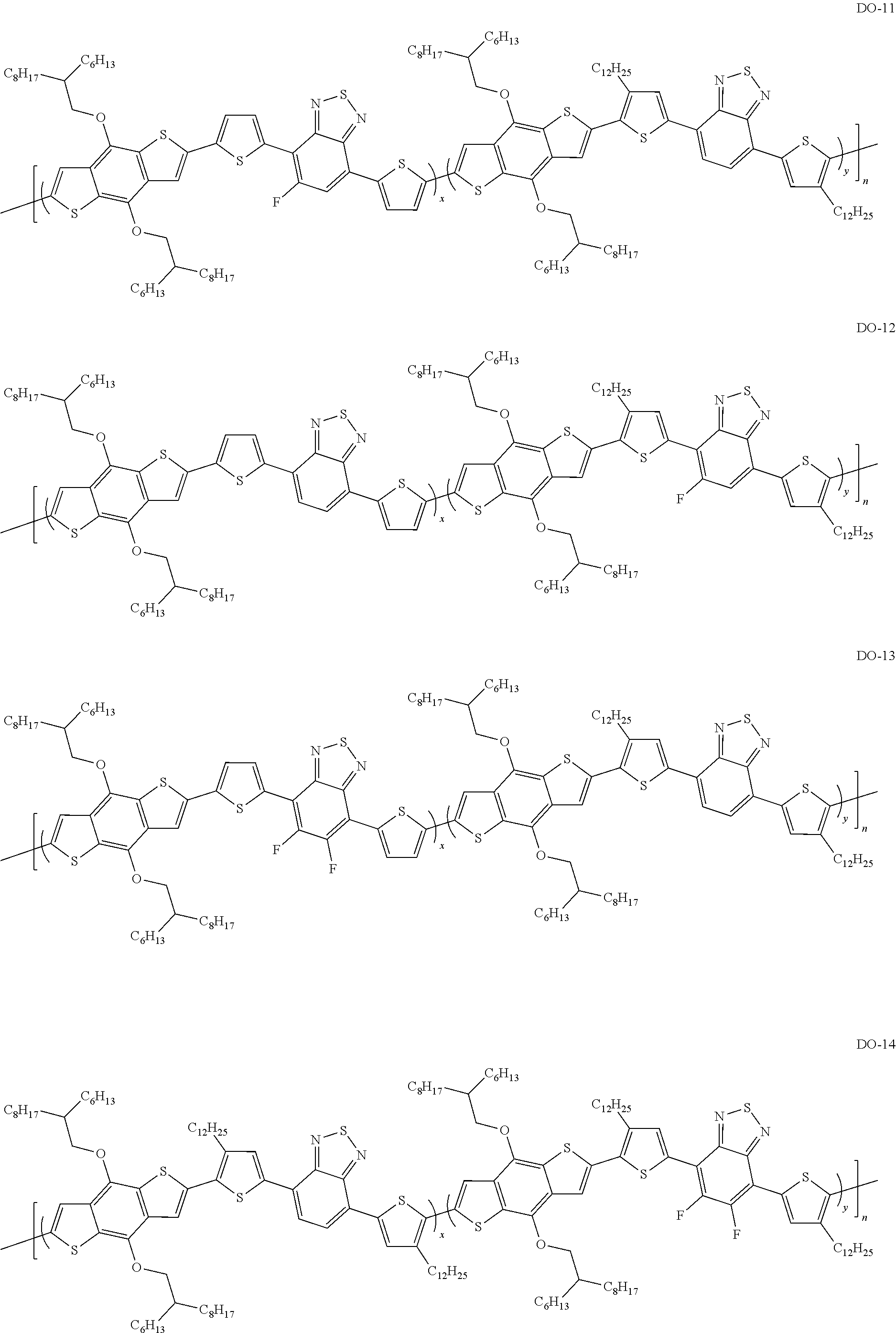

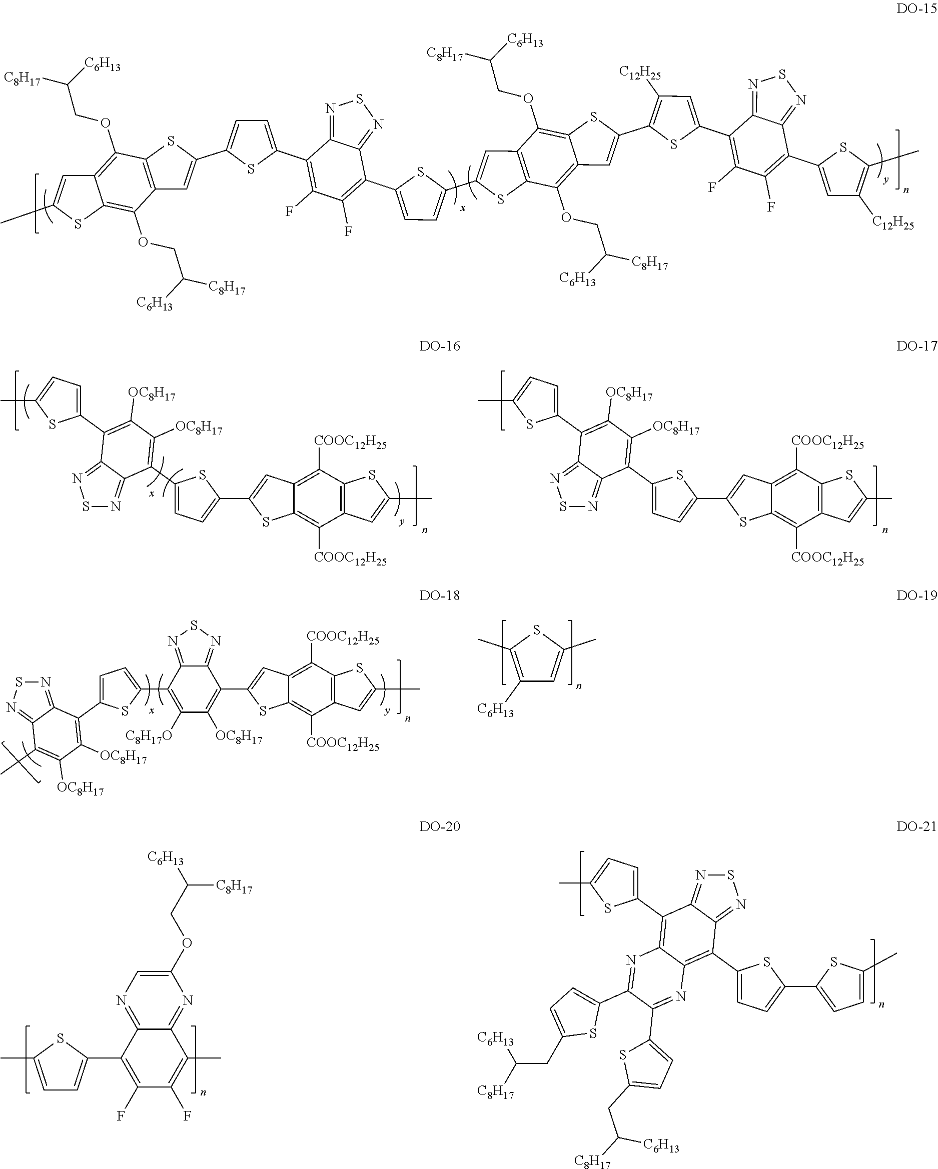

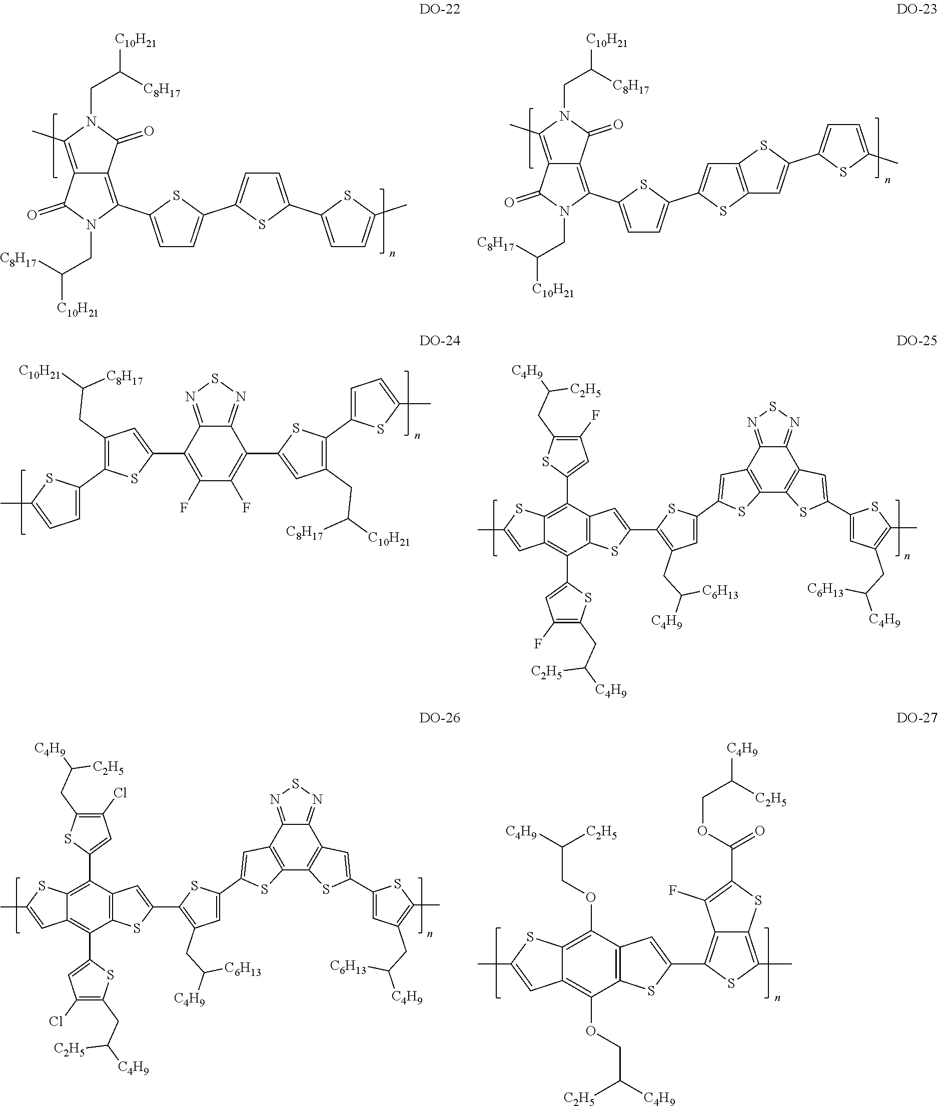

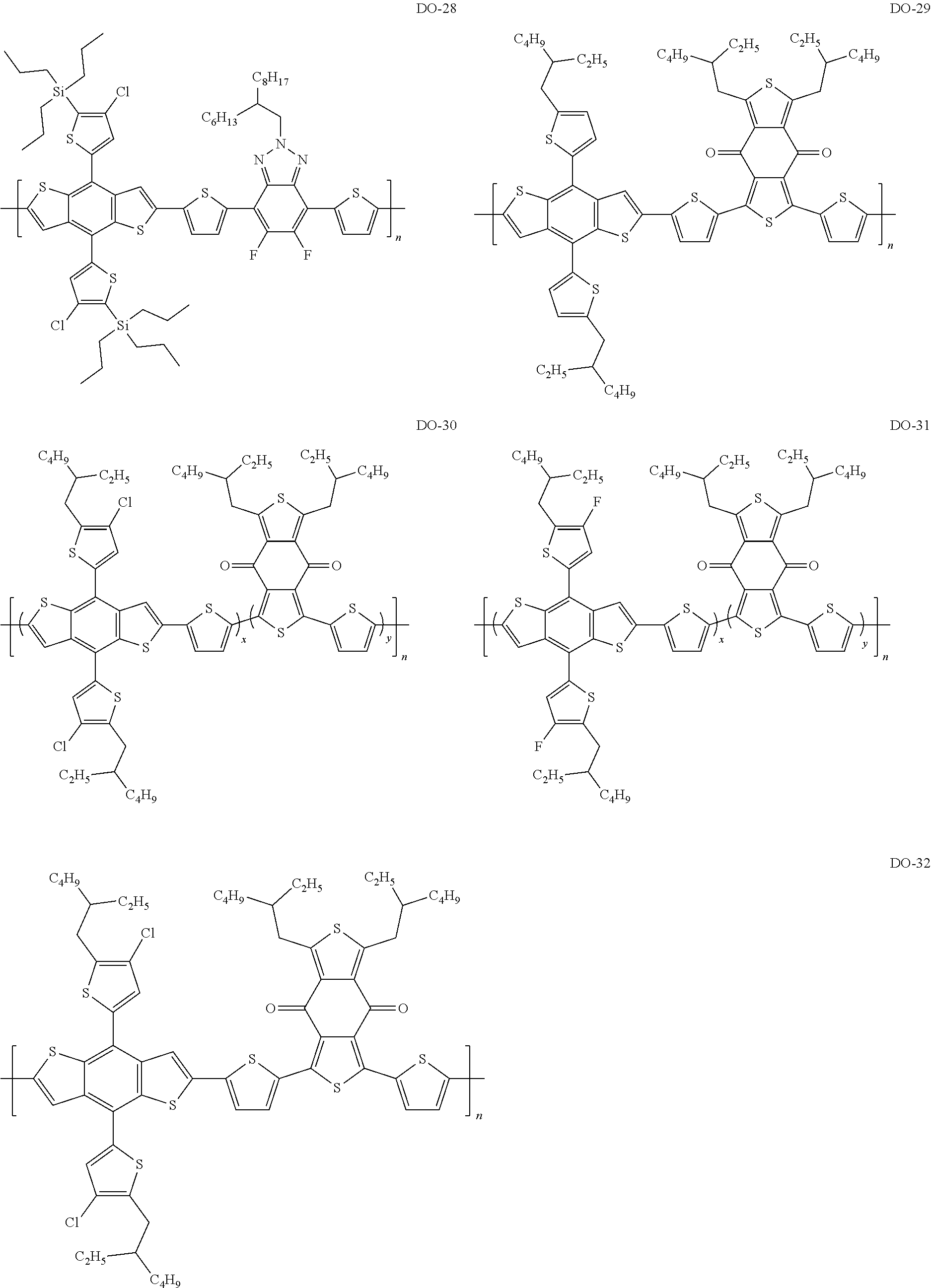

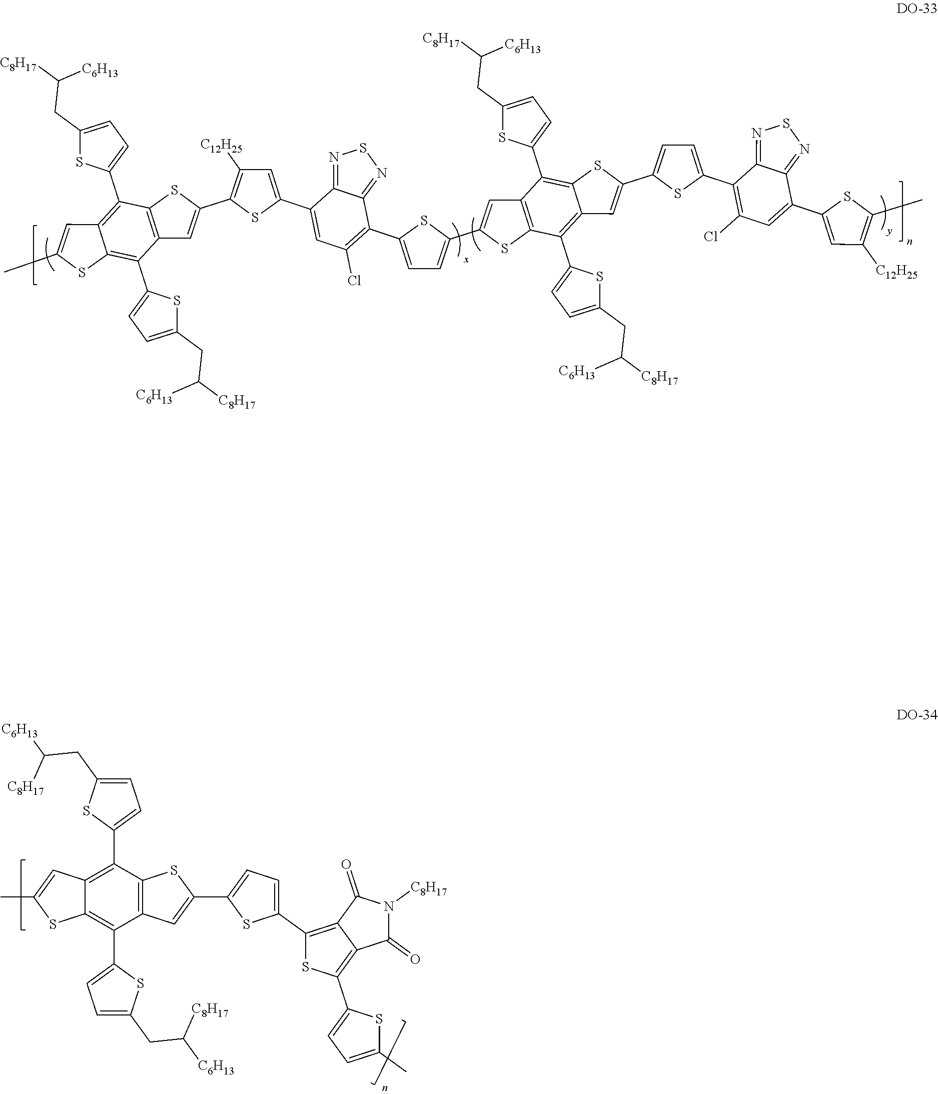

wherein, 0<x<1, 0<y<1, and x+y=1; and n is a positive integer greater than 1.

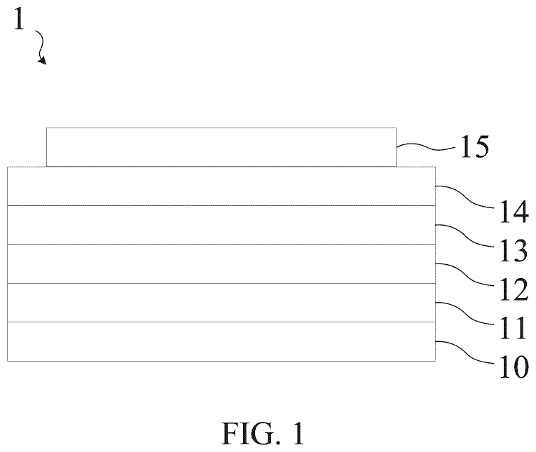

Please refer to FIG. 1. FIG. 1 shows a schematic structural diagram of one embodiment of an organic optoelectronic device of the present invention. As shown in FIG. 1, in another embodiment, the present invention further provides an organic optoelectronic device 1, which comprises a first electrode 11, a second electrode 15 and an active layer 13. The active layer 13, which comprises at least one of the aforementioned organic optoelectronic compounds comprising Formula I and the aforementioned active layer material, is disposed between the first electrode 11 and the second electrode 15. The organic optoelectronic device 1 further comprises a first carrier transporting layer 12 and a second carrier transporting layer 14. The organic optoelectronic device 1 may have a stacked structure, which sequentially includes a substrate 10, the first electrode 11 (transparent or semi-transparent electrode), the first carrier transporting layer 12, the active layer 13, the second carrier transporting layer 14 and the second electrode 15. The first carrier transporting layer 12 is configured to transport carriers in the first electrode 11 and the active layer 13, and the second carrier transporting layer 14 is configured to transport carriers in the active layer 13 and the second electrode 15. Specifically, the first carrier transporting layer 12 is one of an electron transporting layer and a hole transporting layer, and the second carrier transporting layer 14 is the other one. In detail, when the first carrier transporting layer 12 is the electron transport layer, the second carrier transporting layer 14 is the hole transport layer, which is an inverted stacked structure; when the first carrier transporting layer 12 is the hole transporting layer, the second carrier transporting layer 14 is an electron transporting layer, which is a conventional stacked structure. In practice, the organic optoelectronic device 1 may comprise an organic photovoltaic device, an organic photodetector device, or an organic light emitting diode.

In order to illustrate the organic optoelectronic compound of the present invention more clearly, the following experiments will be performed to illustrate the difference in efficacy between Comparative Example Ref. and the aforementioned Examples N01, N03, N05, N14, N15, D01, S01 and P01 of the present invention. Then, they are further used as first acceptor materials to prepare active layers and organic optoelectronic devices for material testing and device testing.

For the optical physical quality testing part of material testing and device testing, the UV absorption spectrum measurement instrument model is Hitachi UH5700, and the oxidation potential is measured by using cyclic voltammetry with CH Instrument 611E.

### Synthesis of N01:

### Synthesis of N01-A0:

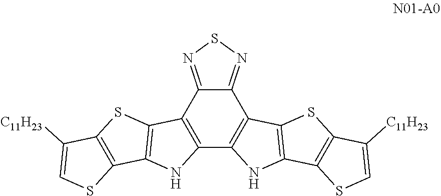

Reference Adv. Mater: 2019, 31, 1807577 synthesis method to prepare N01-A0.

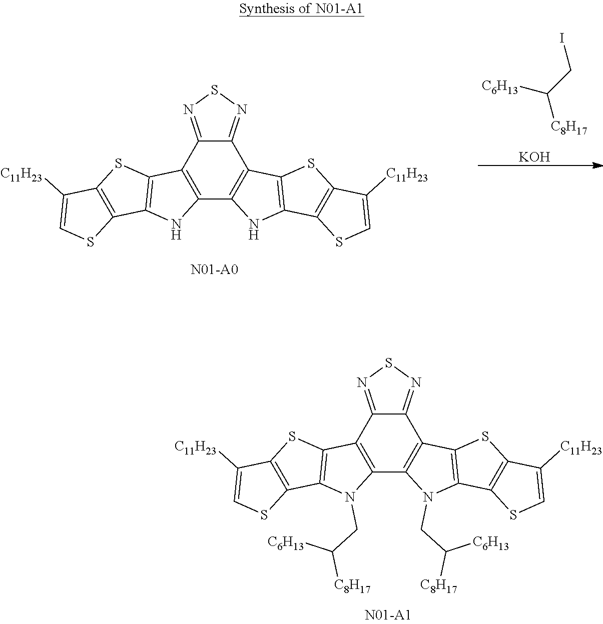

To a solution of N01-A0 (1.50 g, 1.34 mmol) and dimethylsulfoxide (75 mL) and toluene (75 mL) in a 500 mL two necked reaction flask, and then potassium hydroxide (0.90 g, 10.71 mmol) was added into the solution under nitrogen atmosphere. The reaction mixture was heated to 80° C. for 30 minutes. 7-(Iodomethyl)pentadecane (2.10 g, 4.02 mmol) was added dropwise to the reaction mixture. After 3 hours, add another portion of potassium hydroxide (0.90 g, 10.71 mmol) and the second dose of 7-(Iodomethyl)pentadecane (2.10 g, 4.02 mmol) was added after an interval of half an hour. The temperature was kept at 80° C. for 24 hours. The reaction was quenched with deionized water and extracted with heptane. Dry through Magnesium sulfate and concentrated under reduced pressure, and give a brown oil as crude product. The crude product was purified by a silica gel column (the eluent was methylene chloride/heptane=1/19) to obtain product. ¹H NMR (500 MHz, CDCl₃) δ 7.03 (s, 2H), 4.72 (d, J=7.5 Hz, 4H), 2.99 (t, J=7.5 Hz, 4H), 2.19-2.21 (m, 2H), 1.99-2.03 (m, 4H), 0.65-1.52 (m, 98H).

### Synthesis of N01-I1:

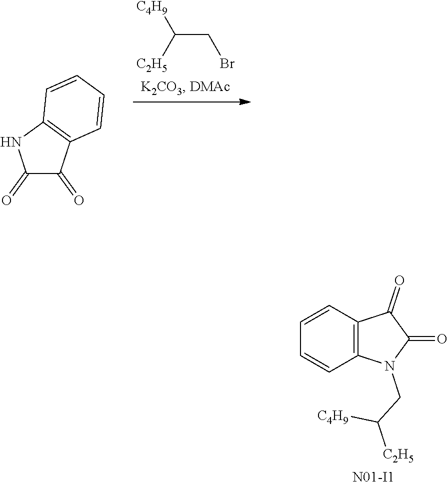

Isatin (2.00 g, 13.59 mmol) was dissolved in 10 mL of dimethylacetamide under nitrogen. Potassium carbonate (2.44 g, 17.67 mmol) was added to the reaction and stirred at room temperature for 30 minutes. 3-(Bromomethyl)hexane (2.89 g, 14.95 mmol) and dimethylacetamide (10 mL) were added into the reaction. The reaction mixture was heated to 50° C. for 18 hours. After cooling, quench reaction by inject deionized water (20 mL) into the reaction mixture and extracted with dichloromethane. The crude product was dried through magnesium sulfate and concentrated in vacuum. The crude product was purified by a silica gel column (the eluant was methylene chloride) to obtain N01-I1 (3.32 g, yield 94%). ¹H NMR (600 MHz, CDCl₃) δ 7.60 (d, J=7.2 Hz, 1H), 7.58 (dt, J=7.8, 1.2 Hz, 1H), 7.11 (t, J=7.8 Hz, 1H), 6.87 (d, J=7.8 Hz, 1H), 3.57-3.64 (m, 2H), 1.79-1.83 (m, 1H), 1.25-1.43 (m, 8H), 0.93 (t, J=7.2 Hz, 3H), 0.89 (t, J=7.2 Hz, 3H).

### Synthesis of N01-A3:

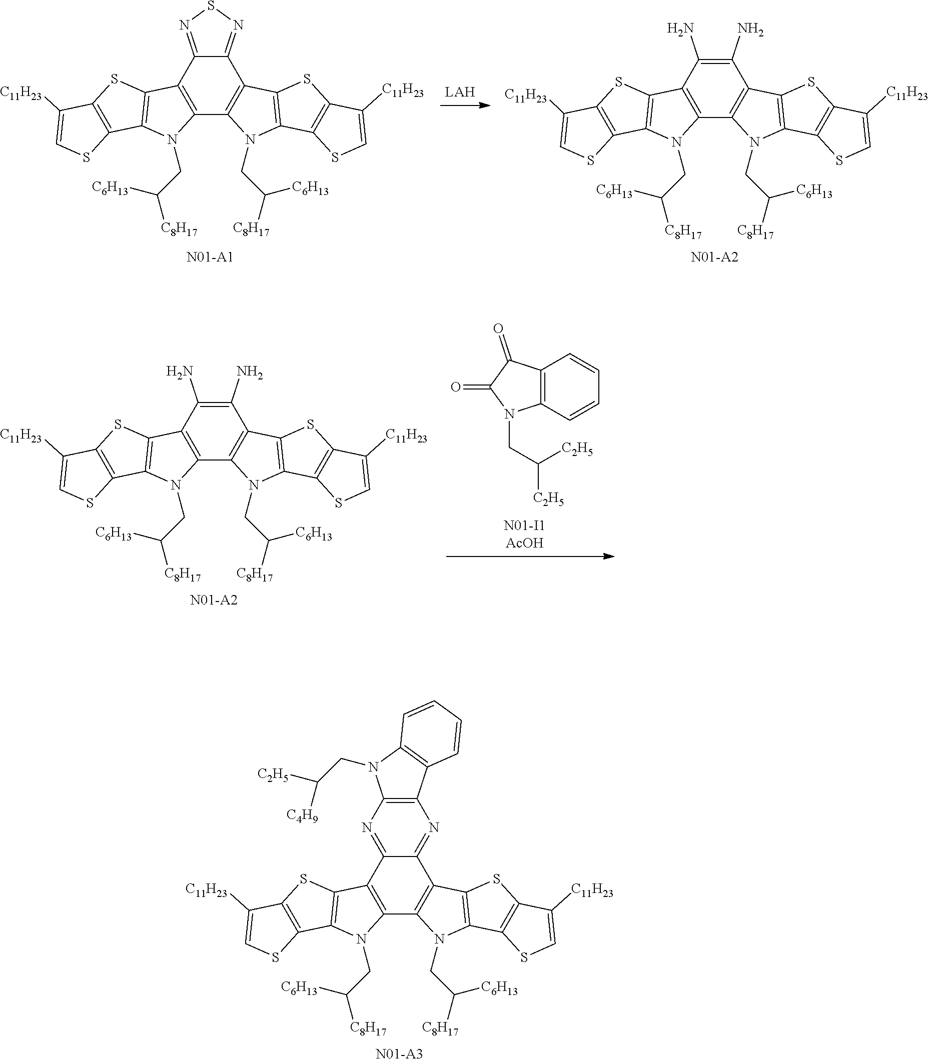

N01-A1 (2.00 g, 1.67 mmol) and tetrahydrofuran (40 mL) was placed into a two necked flask under argon atmosphere at room temperature. The reaction mixture was cooled to 5° C. and lithium aluminum hydride (0.32 g, 8.43 mmol) was added. The reaction mixture was returned to room temperature slowly and stirred under argon for 16 hours. The temperature of reaction mixture was cooled to 5° C. again and quench by injected deionized water (1.00 mL) into the mixture slowly. After stirring for 30 minutes, the reaction mixture was dried through magnesium sulfate. The crude product was purified by a silica gel column to obtain the brown-black oily crude product N01-A2. The dried crude product N01-A2 was directly subjected to the next ring-closure reaction without purification. To a mixture of N01-A2 crude product (1.68 g) and N01-I1 (0.65 g, 2.51 mmol) in a 100 mL flask, and then add glacial acetic acid (35 mL). Under nitrogen atmosphere, the reaction mixture was heated to 120° C. for 2 hours, then the reaction mixture was poured into deionized water (50 mL), and extracted with dichloromethane and heptane with volume ratio 1:3. The extract was neutralized with saturated potassium carbonate aqueous solution until the pH value was between 6 and 7, and then the organic layer was washed with 50 mL of deionized water. After drying in vacuum, the mixture was purified by a silica gel column (the eluent was methylene chloride/heptane=1/1) to obtain N01-A3 (0.62 g, yield after two-step reaction: 31%). ¹H NMR (500 MHz, CDCl₃) δ 8.78 (dd, J=8.0, 1.5 Hz, 1H), 7.59 (dt, J=7.0, 1.5 Hz, 1H), 7.37-7.42 (m, 2H), 7.01 (s, 1H), 6.97 (s, 1H), 4.41-4.67 (m, 6H), 2.81-2.87 (m, 4H), 2.17-2.23 (m, 2H), 1.87-1.93 (m, 6H), 0.67-1.66 (m, 112H).

### Synthesis of N01-A4:

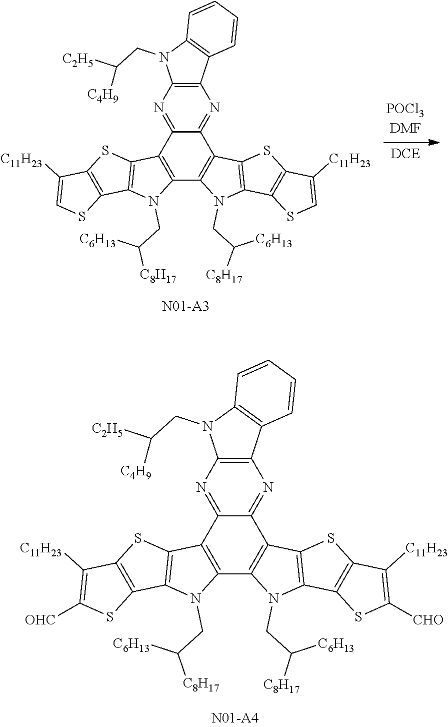

N01-A3 (0.22 g, 0.16 mmol) was placed into a two necked flask and 1,2-dichloroethane (8.0 mL) was added under argon. Dimethylformamide (0.15 ml, 1.90 mmol) and phosphorus oxychloride (0.14 ml, 1.48 mmol) were formulated into a Vilsmeier-Haack reagent. The Vilsmeier-Haack reagent was added to the reaction mixture and then heated to 70° C. for 4 hours. The reaction mixture was cooling to room temperature and stirred with 2% potassium carbonate aqueous solution for more than 1 hour. The reaction mixture was extracted with heptane and dichloromethane, the obtained extract was washed with deionized water and then concentrated and dried. The crude product was purified by silica gel column (the eluent was dichloromethane and heptane) to obtain N01-A4 (0.21 g, yield 91%). ¹H NMR (500 MHz, CDCl₃) δ 10.15 (s, 1H), 10.14 (s, 1H), 8.78 (dd, J=9.5, 1.5 Hz, 1H), 7.66 (dt, J=7.0, 1.5 Hz, 1H), 7.42-7.47 (m, 2H), 4.41-4.64 (m, 6H), 3.23-3.21 (m, 4H), 2.14-2.17 (m, 2H), 1.96-1.98 (m, 4H), 1.82-1.87 (m, 2H), 0.67-1.61 (m, 112H).

### Synthesis of N01:

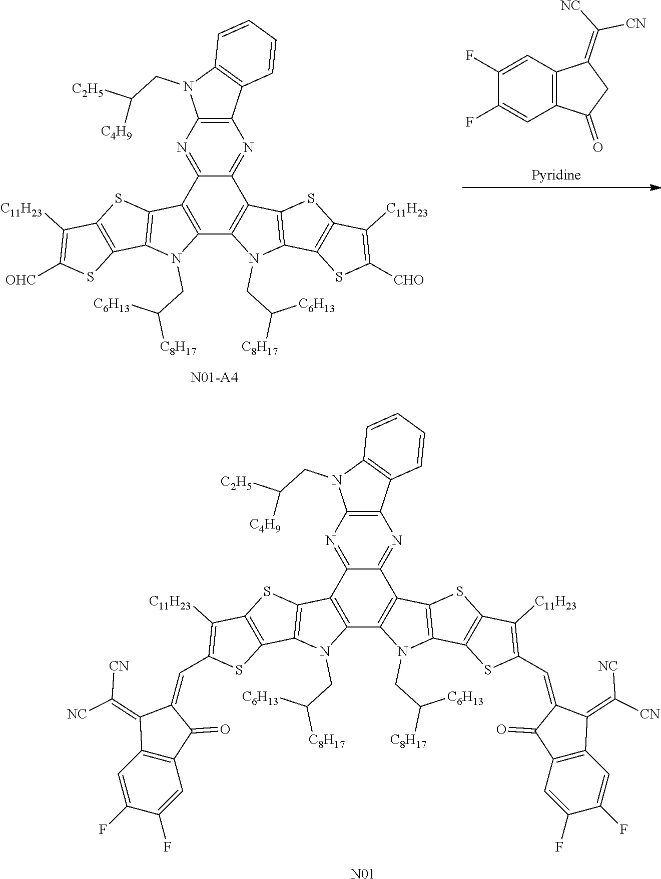

To a solution of N01-A4 (0.17 g, 0.12 mmol), 5,6-Difluoro-1H-indene-1,3(2H)-dione (0.11 g, 0.48 mmol), and chloroform (8.8 mL) in a two necked flask under argon. Pyridine (0.17 ml, 2.11 mmol) was added at room temperature and the reaction mixture was heated to 65° C. for 4 hours. After cooling to room temperature, 17 mL of methanol was injected into the mixture to precipitate the product. The solid was collected by filtration and purified by a silica gel column (the eluent was chloroform and heptane), and then the solid was precipitated with isopropyl alcohol to obtain N01 (0.17 g, yield 75%). ¹H NMR (600 MHz, CDCl₃) δ 9.19 (s, 1H), 9.18 (s, 1H), 8.78 (dd, J=7.8, 1.2 Hz, 1H), 8.55-8.59 (m, 2H), 7.67-7.70 (m, 3H), 7.45-7.49 (m, 2H), 4.76 (d, J=7.8 Hz, 1H), 4.72 (d, J=7.2 Hz, 1H), 4.40-4.56 (m, 2H), 3.25-3.27 (m, 4H), 2.14-2.17 (m, 2H), 1.89-1.93 (m, 6H), 0.51-1.6 (m, 112H).

### Synthesis of N14:

### Synthesis of N14-IB1:

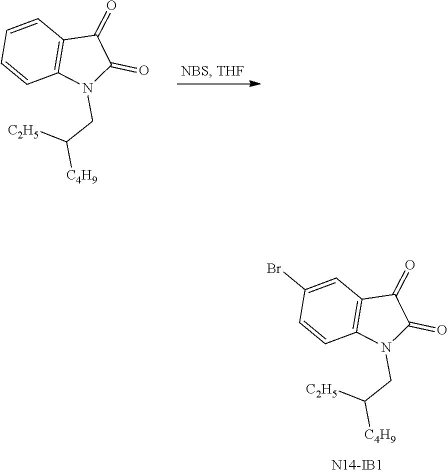

N01-I1 (1.06 g, 4.09 mmol) was placed into a 100 mL two neck reaction flask, and tetrahydrofuran (40 mL) was added under nitrogen. N-bromosuccinimide (800 mg, 4.50 mmol) was added at room temperature and the reaction mixture was stirred for 18 hours. The reaction was quenched with saturated aqueous potassium carbonate solution, and then extracted with dichloromethane, the organic layer solution was extracted and washed with deionized water. After dry through magnesium sulfate, the product was purified by silica gel column (the eluent was methylene chloride and heptane). N14-IB1 (1.12 g, yield 81%) was obtained after concentration. ¹H NMR (500 MHz, CDCl₃) δ 7.70 (d, J=2.0 Hz, 1H), 7.68 (dd, J=7.0, 1.5 Hz, 1H), 6.78 (d, J=7.5 Hz, 1H), 3.56-3.63 (m, 2H), 1.76-1.80 (m, 1H), 1.22-1.39 (m, 8H), 0.93 (t, J=6.5 Hz, 3H), 0.88 (t, J=6.0 Hz, 3H).

### Synthesis of N14-A1:

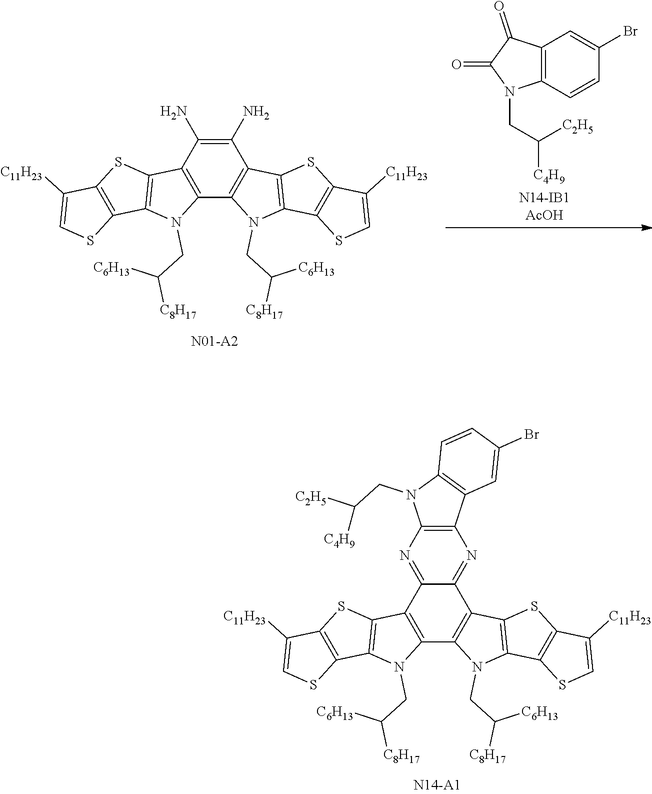

N14-A1 (0.35 g, yield 48%) was synthesized according to the N01-A3 procedure, and the crude N01-A2 (0.57 g, 0.49 mmol) and N14-IB1 (0.17 g, 0.51 mmol) were used as starting materials. ¹H NMR (500 MHz, CDCl₃) δ 8.89 (d, J=2.0 Hz, 1H), 7.65 (t, J=7.0 Hz, 1H), 7.25 (t, J=7.0 Hz, 1H), 7.02 (s, 1H), 6.98 (s, 1H), 4.59-4.63 (m, 4H), 4.38-4.50 (m, 2H), 2.85-2.88 (m, 4H), 2.09-2.11 (m, 2H), 1.89-1.95 (m, 6H), 0.67-1.60 (m, 112H).

### Synthesis of N14-A2:

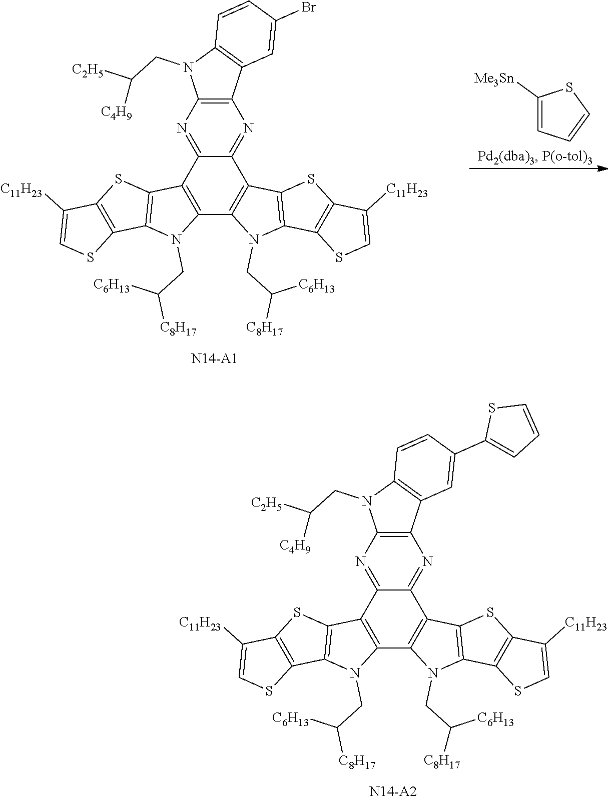

N14-A1 (0.30 g, 0.21 mmol), 2-(tributylstannyl)thiophene (0.17 g, 0.45 mmol), tris(dibenzylideneacetone)dipalladium (0.01 g, 0.01 mmol) and tris(o-methylphenyl)phosphine (0.013 g, 0.04 mmol) were placed in a 100 mL two necked flask under argon, and then injected deoxygenated toluene (30 mL) into the reaction mixture. The reaction mixture was placed in a 90° C. oil bath and heated for 18 hours. After cooling to room temperature, the reaction product was filtered through celite and concentrated to obtain a brown crude product. The crude product was purified by silica gel column (the eluent was heptane and dichloromethane) to obtain N14-A2 (0.26 g, yield 84%). ¹H NMR (500 MHz, CDCl₃) δ 8.99 (d, J=2.5 Hz, 1H), 7.84 (dd, J=9.0, 2.0 Hz, 1H), 7.56 (s, 1H), 7.37 (d, J=2.5 Hz, 1H), 7.36 (d, J=1.0 Hz, 1H), 7.18 (d, J=1.0 Hz, 1H), 7.01 (s, 1H), 6.97 (s, 1H), 4.57-4.61 (m, 4H), 4.42-4.59 (m, 2H), 2.84-2.88 (m, 4H), 2.37-2.15 (m, 2H), 1.89-1.91 (m, 6H), 0.79-1.61 (m, 112H).

### Synthesis of N14-A3:

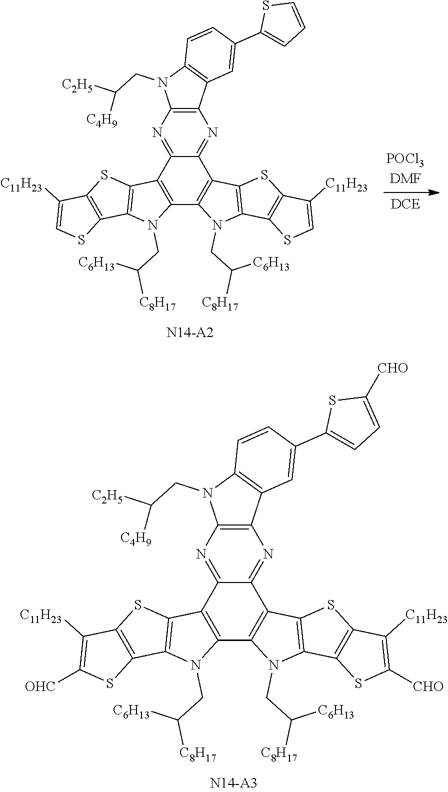

N14-A2 (0.18 g, 0.12 mmol) and 7.0 mL of 1,2-dichloroethane were placed in a 50 mL two necked flask under argon atmosphere at room temperature. Dimethylformamide (0.51 mL, 6.58 mmol) and phosphorus oxychloride (0.17 mL, 1.77 mmol) were reacted to form the Vilsmeier-Haack reagent and then added to the reactant slowly. The reaction mixture was heated to 70° C. for 4 hours. The reaction mixture was cooled to room temperature and stirred with 2% aqueous potassium carbonate solution for more than 1 hour. The reaction mixture was extracted with heptane and dichloromethane, and the obtained extract was washed with deionized water, and then dried and concentrated. The crude product was purified by silica gel column (the eluent was methylene chloride and heptane) to obtain orange solid product N14-A3 (0.18 g, yield 95%). ¹H NMR (500 MHz, CDCl₃) δ 10.18 (s, 1H), 10.16 (s, 1H), 8.91 (s, 1H), 8.10 (s, 1H), 7.73 (dd, J=9.0, 2.5 Hz, 1H), 7.29 (d, J=9.0 Hz, 1H), 4.70-4.62 (m, 4H), 4.60-4.36 (m, 2H), 3.28-3.18 (m, 4H), 2.17-2.10 (m, 2H), 1.97-1.95 (m, 6H), 0.66-1.73 (m, 112H).

### Synthesis of N14:

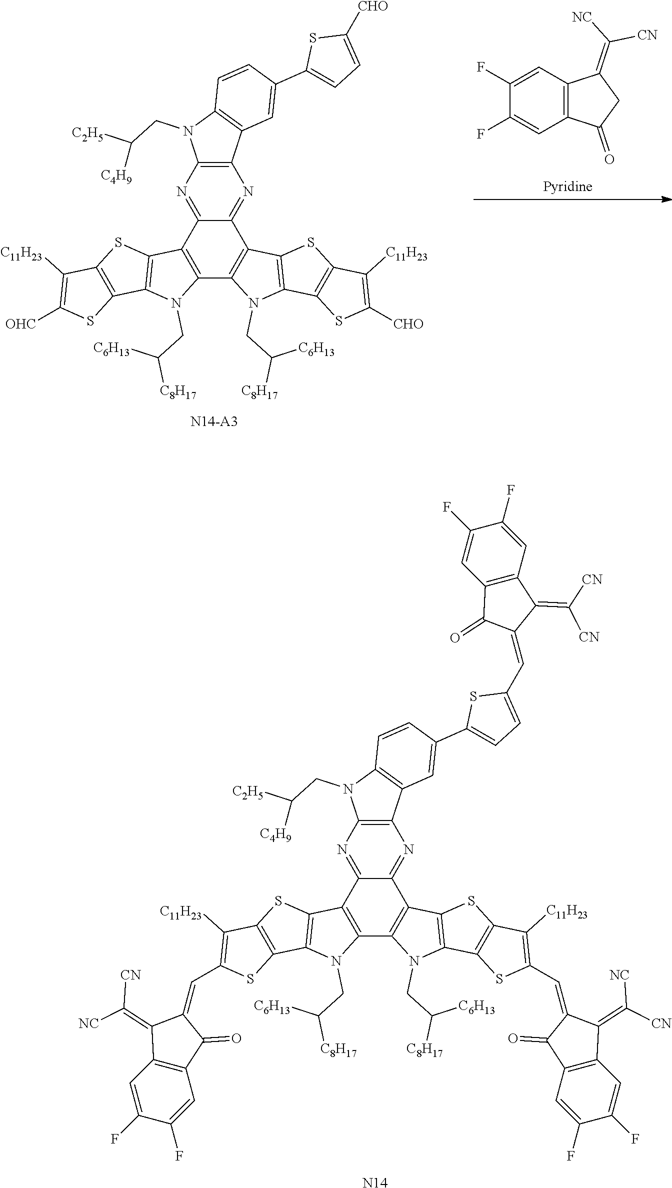

N14-A3 (0.18 g, 0.11 mmol), 5,6-Difluoro-1H-indene-1,3 (2H)-dione (0.17 g, 0.74 mmol), and 12 mL of chloroform were placed into a two necked flask under argon atmosphere. 0.18 mL of pyridine was added at room temperature and the mixture was heated to 65° C. for 5 hours. After cooling to room temperature, 40 mL of methanol was added to precipitate the product, and the solid was collected by filtration. The crude product was purified by silica gel column (the eluent was chloroform and heptane), and then the solid was precipitated with isopropyl alcohol to obtain N14 (0.22 g, yield 89%). ¹H NMR (600 MHZ, CDCl₃) δ 9.20 (s, 1H), 9.15 (s, 1H), 9.01 (br, 1H), 8.85 (br, 1H), 8.61-8.58 (m, 2H), 8.50 (br, 1H), 7.99 (br, 1H), 7.81 (s, 1H), 7.77-7.67 (m, 4H), 4.84-4.79 (m, 4H), 4.42-4.60 (m, 2H), 3.25-3.30 (m, 4H), 2.10-2.07 (m, 2H), 1.89-1.95 (m, 6H), 0.66-1.59 (m, 112H).

### Synthesis of N15:

### Synthesis of N15-A0:

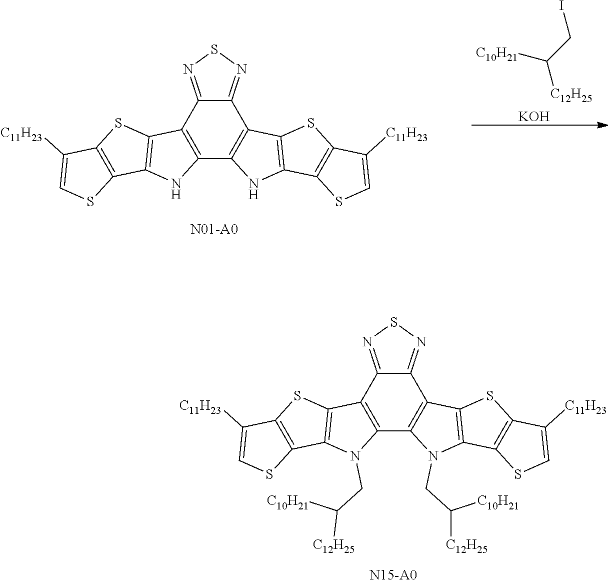

N15-A0 (2.65 g, yield 92%) was synthesized according to the N01-A1. N01-A0 (1.5 g, 1.34 mmol) and 1-iodo-2-decyltetradecane (5.6 g, 4.12 mmol) were used as starting materials. ¹H NMR (500 MHz, CDCl₃) δ 7.02 (s, 2H), 4.71 (d, J=7.5 Hz, 4H), 3.00 (t, J=7.5 Hz, 4H), 2.19-2.21 (m, 2H), 1.99-2.03 (m, 4H), 0.63-1.60 (m, 134H).

### Synthesis of N15-A2:

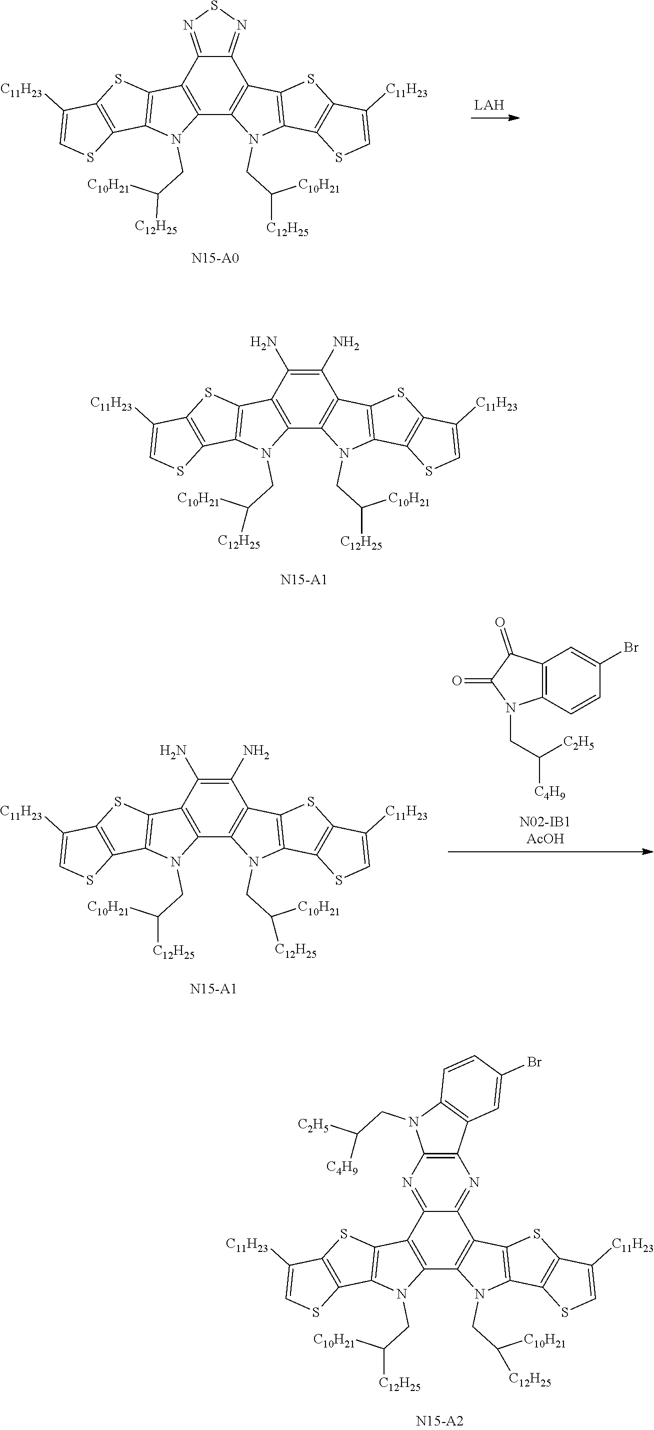

N15-A2 (134 mg, yield 40%) was synthesized according to the procedure of N01-A2 and N14-A2. N15-A0 (0.38 g) was used as a staring material. ¹H NMR (600 MHz, CDCl₃) δ 8.88 (d, J=2.4 Hz, 1H), 8.10 (s, 1H), 7.67 (dd, J=9.0, 2.4 Hz, 1H), 7.56 (s, 1H), 7.25 (d, J=2.4 Hz, 1H), 7.00 (s, 1H), 6.96 (s, 1H), 4.56-4.60 (m, 4H), 4.38-4.48 (m, 2H), 2.84-2.88 (m, 4H), 2.08-2.10 (m, 4H), 0.79-1.60 (m, 146H).

### Synthesis of N15-A3:

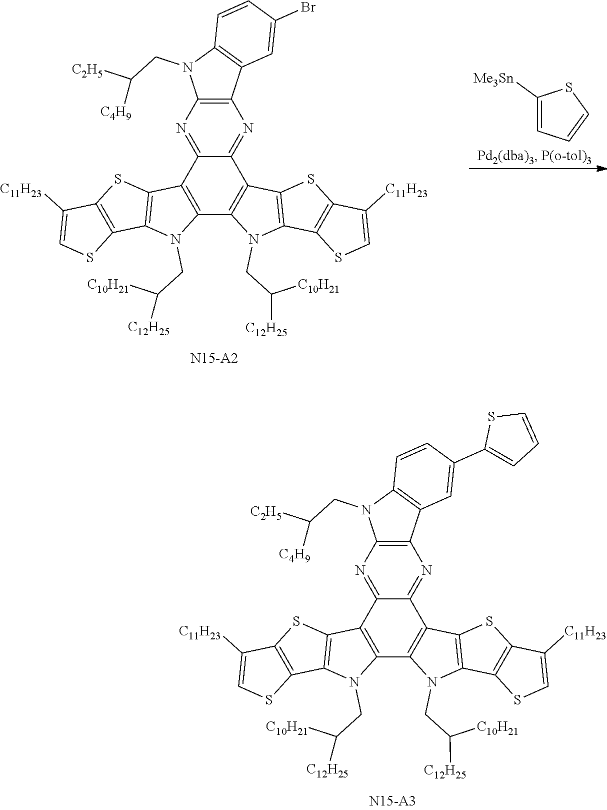

N15-A3 (74 mg, yield 62%) was synthesized according to the procedure of N14-A3. 120 mg of N15-A2 was used as a staring material. ¹H NMR (600 MHz, CDCl₃) δ 8.98 (d, J=1.8 Hz, 1H), 7.84 (dd, J=9.0, 2.4 Hz, 1H), 7.56 (d, J=3.0 Hz, 1H), 7.40 (d, J=8.4 Hz, 1H), 7.37 (d, J=7.2 Hz, 1H), 7.18 (dd, J=3.6, 1.8 Hz, 1H), 7.00 (s, 1H), 6.97 (s, 1H), 4.61-4.58 (m, 4H), 4.53-4.42 (m, 2H), 2.84-2.88 (m, 4H), 2.16-2.14 (m, 2H), 1.64-0.83 (m, 146H).

### Synthesis of N15-A4:

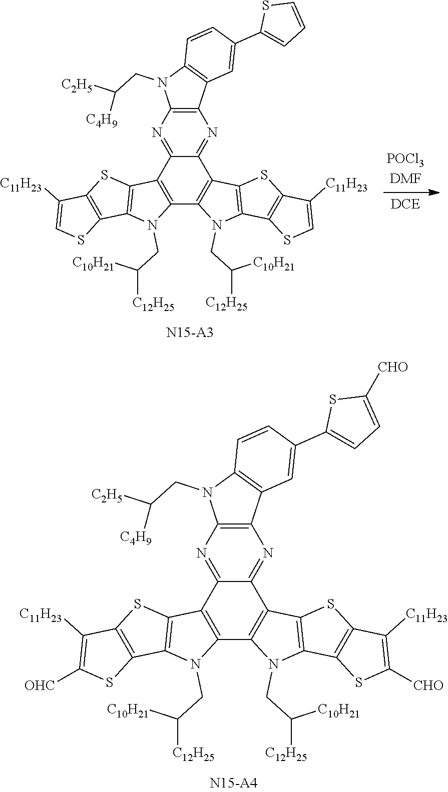

N15-A4 (47 mg, yield 40%) was synthesized according to N14-A4 procedure. 112 mg of N15-A3 was used as a staring material. ¹H NMR (500 MHz, CDCl₃) δ 10.16 (s, 1H), 10.15 (s, 1H), 9.98 (s, 1H), 9.05 (d, J=1.5 Hz, 1H), 7.94 (dd, J=7.0, 1.5 Hz, 1H), 7.85 (d, J=3.0 Hz, 1H), 7.67 (d, J=3.0 Hz, 1H), 7.50 (d, J=7.5 Hz, 1H), 4.63-4.42 (m, 6H), 3.27-3.22 (m, 4H), 2.17-2.13 (m, 2H), 2.00-1.96 (m, 4H), 1.89-1.82 (m, 2H), 0.66-1.64 (m, 142H).

### Synthesis of N15:

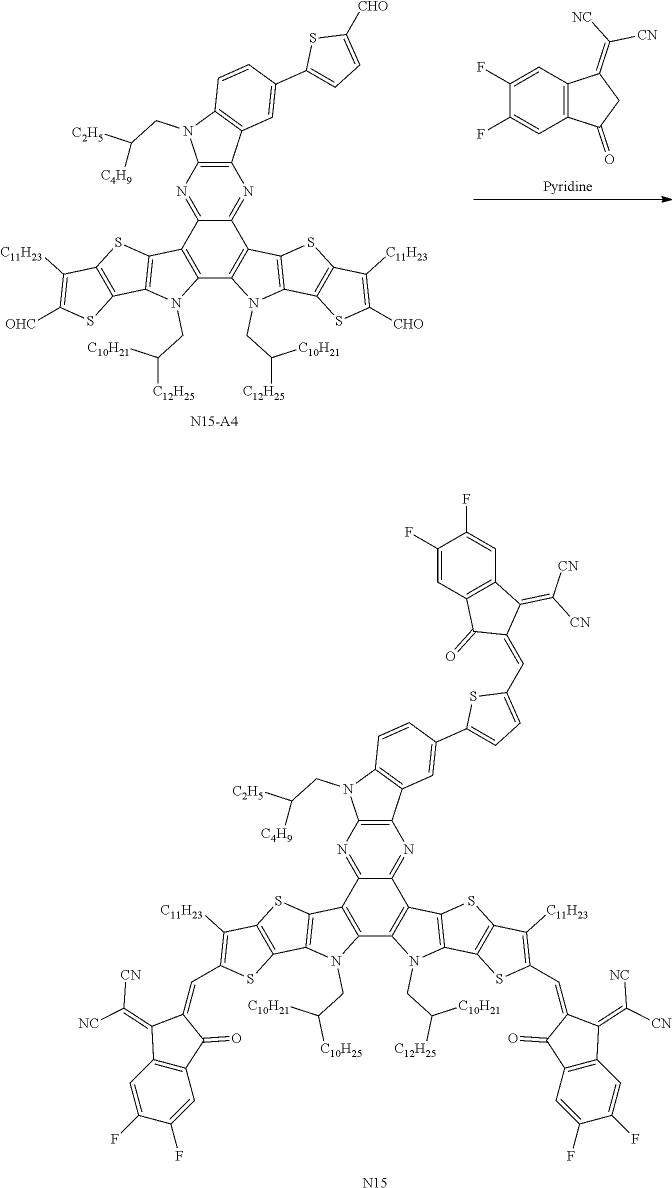

N15 (51 mg, yield 80%) was synthesized according to N14 procedure. 47 mg of N15-A4 was used as a starting material. ¹H NMR (600 MHz, CDCl₃) δ 9.20 (s, 2H), 9.14 (s, 1H), 8.99 (br, 1H), 8.84 (br, 1H), 8.61-8.57 (m, 2H), 8.50 (br, 1H), 7.99 (br, 1H), 7.81 (s, 1H), 7.77-7.67 (m, 4H), 4.87-4.74 (m, 4H), 4.64-4.41 (m, 2H), 3.23-3.30 (m, 4H), 2.11-2.06 (m, 2H), 1.96-1.87 (m, 6H), 0.66-1.59 (m, 142H).

### Synthesis of N03:

### Synthesis of N03-A1:

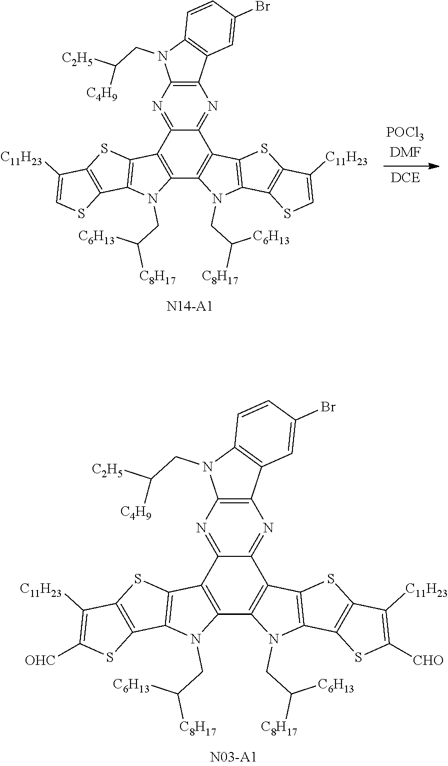

N14-A1 (0.20 g, 0.14 mmol) and 8.0 mL of 1,2-dichloroethane were placed in a 50 mL two necked flask under argon. Dimethylformamide 0.25 mL and phosphorus oxychloride 0.25 mL were reacted to form the Vilsmeier-Haack reagent and then slowly added to the reactant. The reaction mixture was heated to 55° C. for 2 hours. The reaction mixture was cooled to room temperature and stirred with 2% aqueous potassium carbonate solution for more than 1 hour. The reaction mixture was extracted with heptane and dichloromethane, and the obtained extract was washed with deionized water, and then dried and concentrated. The crude product was purified by silica gel column (the eluent was dichloromethane and heptane) to obtain N03-A1 (0.12 g, yield 56%). ¹H NMR (500 MHz, CDCl₃) δ 10.16 (s, 1H), 10.15 (s, 1H), 8.88 (d, J=2.0 Hz 1H), 8.10 (s, 1H), 7.73 (dd, J=9.0, 2.0 Hz, 1H), 7.29 (d, J=9.0 Hz, 1H), 4.64-4.56 (m, 4H), 4.53-4.37 (m, 2H), 3.25-3.21 (m, 4H), 2.10-2.07 (m, 2H), 1.97-1.93 (m, 6H), 0.66-1.73 (m, 112H).

### Synthesis of N03:

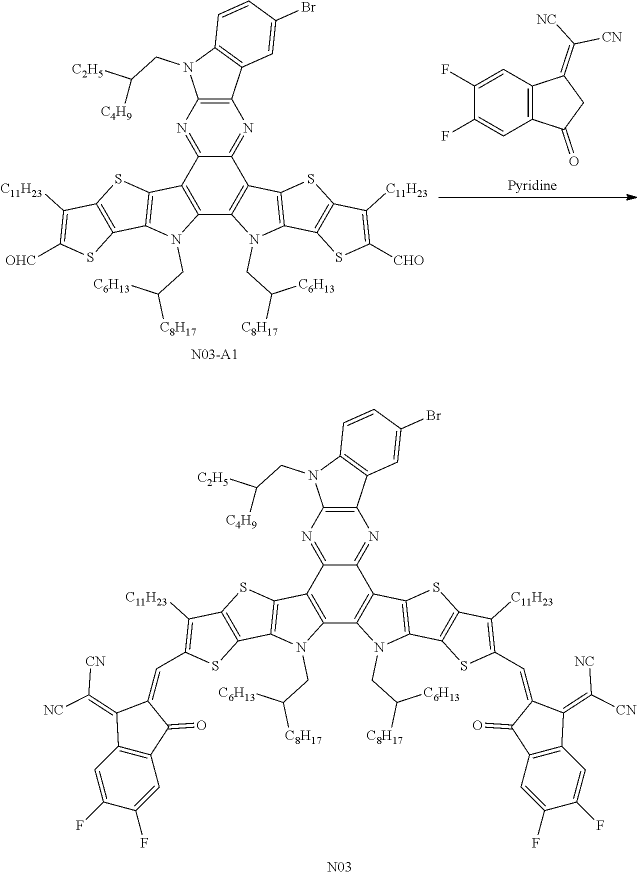

N03-A1 (0.10 g, 0.06 mmol), 5,6-Difluoro-1H-indene-1,3 (2H)-dione (0.06 g, 0.25 mmol), and 7 mL of chloroform were placed into a two necked flask under argon. 0.10 mL of pyridine was added at room temperature and the reaction mixture was heated to 65° C. for 2 hours. After cooling to room temperature, 20 mL of methanol was added to precipitate the product, and the solid was collected by filtration. The crude product was purified by silica gel column (the eluent was chloroform and heptane), and then precipitate the solid with isopropyl alcohol to obtain the black solid product N03 (0.09 g, yield 71%). ¹H NMR (600 MHz, CDCl₃) δ 9.19 (s, 1H), 9.18 (s, 1H), 8.89 (d, J=2.4 Hz, 1H), 8.59-8.56 (m, 2H), 7.77 (dd, J=9.0, 2.4 Hz, 1H), 7.71-7.68 (m, 2H), 7.36 (d, J=9.0 Hz, 1H), 4.76 (d, J=7.8 Hz, 2H), 4.72 (d, J=7.2 Hz, 2H), 4.35-4.60 (m, 2H), 3.26-3.30 (m, 4H), 2.14-2.09 (m, 2H), 1.86-1.95 (m, 6H), 0.64-1.58 (m, 112H).

### Synthesis of N05:

### Synthesis of N05-A1:

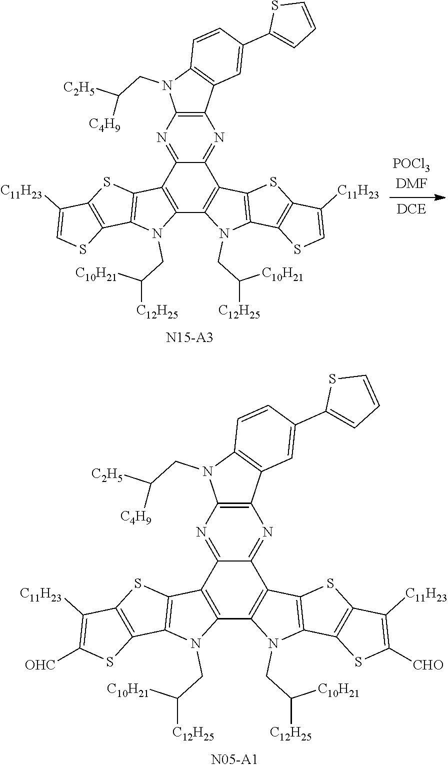

N05-A1 (35 mg, yield 30%) can be obtained according to the procedure of N03-A1. 112 mg of N15-A3 was used as a starting material. ¹H NMR (500 MHz, CDCl₃) δ 10.15 (s, 1H), 10.14 (s, 1H), 8.97 (d, J=1.5 Hz, 1H), 7.89 (dd, J=7.0, 1.5 Hz, 1H), 7.56 (d, J=2.5 Hz, 1H), 7.45 (d, J=7.5 Hz, 1H), 7.39 (d, J=4.0 Hz, 1H), 7.20 (t, J=3.5 Hz, 1H), 4.63-4.40 (m, 6H), 3.25-3.21 (m, 4H), 2.17-2.15 (m, 2H), 2.01-1.94 (m, 4H), 1.89-1.82 (m, 2H), 0.82-1.63 (m, 142H).

### Synthesis of N05:

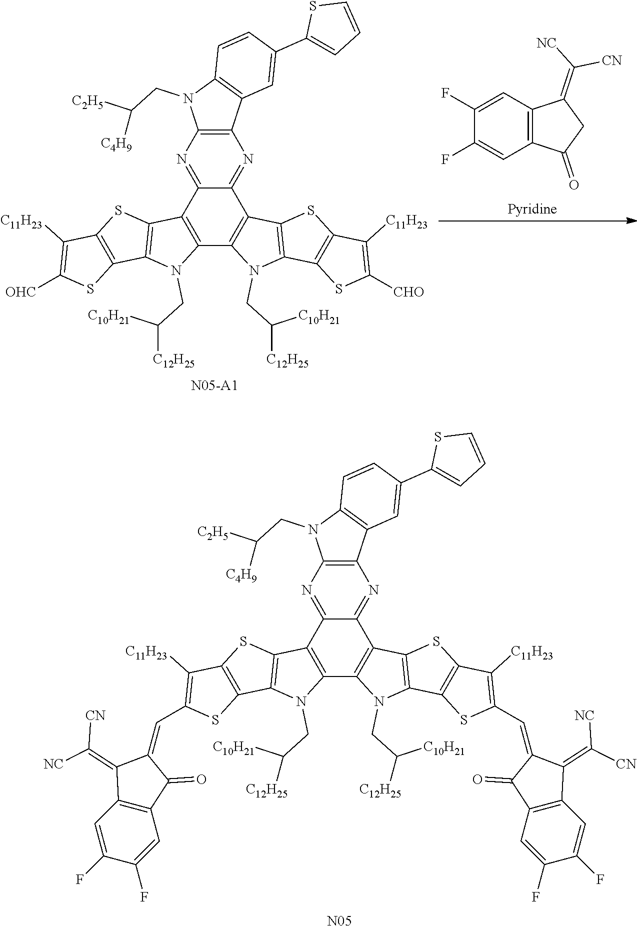

N05 (35 mg, yield 80%) can be obtained according to the procedure of N14. 35 mg of N05-A1 was used as a starting material. ¹H NMR (500 MHz, CDCl₃) δ 9.20 (s, 1H), 9.19 (s, 1H), 8.96 (d, J=2.0 Hz, 1H), 8.60-8.56 (m, 2H), 7.92 (dd, J=8.5, 2.0 Hz, 1H), 7.72-7.68 (m, 2H), 7.57 (dd, J=3.5, 1.0 Hz, 1H), 7.46 (d, J=9.0 Hz, 1H), 7.41 (dd, J=5.5, 1.0 Hz, 1H), 7.21 (dd, J=5.0, 3.5 Hz, 1H), 4.77-4.73 (m, 4H), 4.59-4.39 (m, 2H), 3.31-3.27 (m, 4H), 2.15-2.17 (m, 2H), 1.96-1.88 (m, 6H), 0.64-1.81 (m, 142H).

### Synthesis of D01:

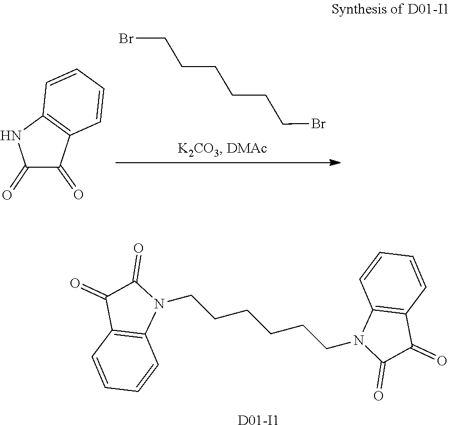 [SMILES: O=C1Nc2ccccc2C1=O ; BrCCCCCCBr ; O=C1C(=O)N(CCCCCCN2C(=O)C(=O)c3ccccc32)c2ccccc21 ; "D01-I1"]

Isatin (1.51 g, 10.26 mmol) was dissolved in 20 mL of dimethylacetamide under nitrogen, potassium carbonate (7.09 g, 51.32 mmol) was added and stirred at room temperature for 30 minutes. 1,6-Dibromohexane (0.79 mL, 5.13 mmol) and 10 mL of dimethylacetamide were slowly added. The mixture was heated to 60° C. and reacted for 24 hours. After cooling, 150 mL of deionized water was injected to terminate the reaction. The solid was filtered and collected, and washed with deionized water and methanol. After drying, the orange-red solid product D01-I1 (1.60 g, yield 83%) was obtained. ¹H NMR (600 MHz, CDCl₃) δ 7.57-7.61 (m, 4H), 7.11 (dt, J=7.8, 1.2 Hz, 2H), 6.88 (d, J=8.4 Hz, 2H), 3.72 (t, J=7.2 Hz, 4H), 1.72-1.70 (m, 4H), 1.45-1.43 (m, 4H).

### Synthesis of D01-A1:

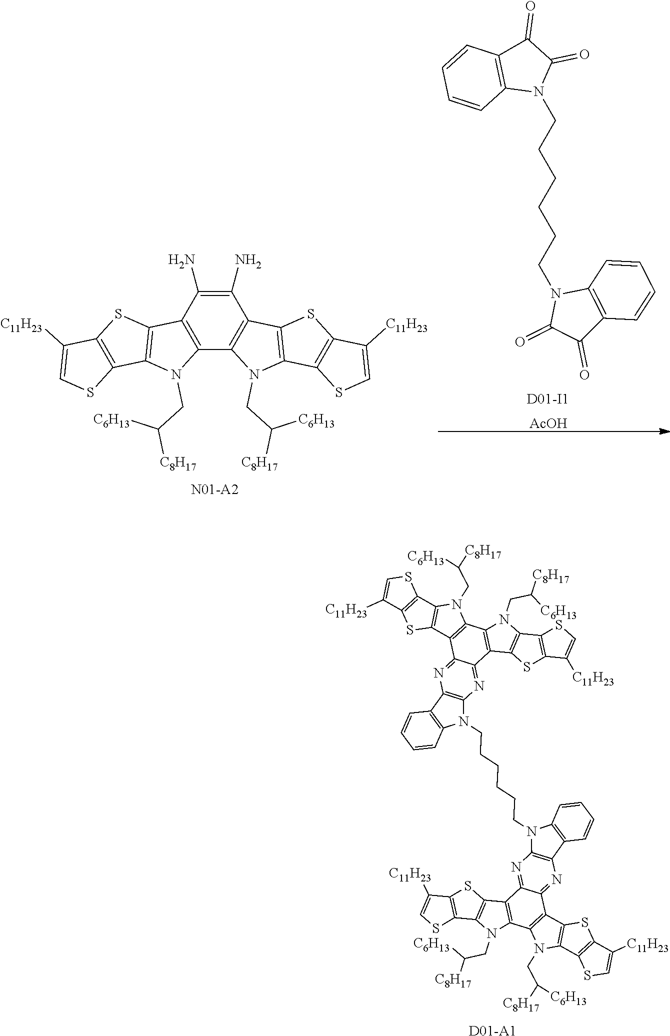

D01-A1 (0.20 g, yield 30%) was synthesized according to the procedure of N01-A3. N01-A2 (0.59 g, 0.50 mmol) and D01-I1 (0.1 g, 0.25 mmol) were used as starting materials. ¹H NMR (500 MHz, CDCl₃) δ 8.76 (d, J=8.0 Hz, 2H), 7.46 (t, J=7.0 Hz, 2H), 7.38-7.34 (m, 4H), 7.01 (s, 2H), 6.96 (s, 2H), 4.71-4.58 (m, 12H), 2.90-2.83 (m, 8H), 2.00-2.10 (m, 4H), 1.89-1.87 (m, 16H), 0.67-1.42 (m, 196H).

### Synthesis of D01-A2:

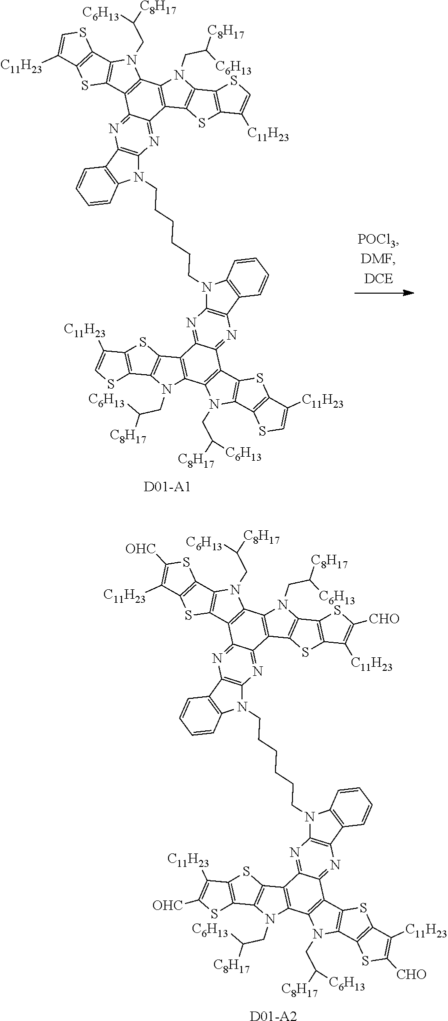

D01-A1 (0.20 g, 0.08 mmol) and 8.0 mL of 1,2-dichloroethane were placed into a 50 mL two necked flask under argon. Dimethylformamide 1.42 mL and phosphorus oxychloride 1.43 mL were reacted to form the Vilsmeier-Haack reagent and then slowly added to the reactant. The reaction mixture was heated to 70° C. for 24 hours. The reaction mixture was cooled to room temperature and stirred with 2% aqueous potassium carbonate solution for more than 1 hour. The reaction mixture was extracted with heptane and dichloromethane, and the obtained extract was washed with deionized water, and then dried and concentrated. The crude product was purified by silica gel column (the eluent was ethyl acetate and heptane) to obtain orange solid product D01-A2 (0.20 g, yield 94%). ¹H NMR (600 MHz, CDCl₃) δ 10.15 (s, 2H), 10.12 (s, 2H), 8.77 (dd, J=7.8, 1.2 Hz, 2H), 7.54 (td, J=8.4, 1.2 Hz, 2H), 7.41-7.39 (m, 4H), 4.64-4.60 (m, 8H), 3.24-3.18 (m, 8H), 2.06-2.04 (m, 4H), 1.97-1.82 (m, 16H), 0.66-1.73 (m, 200H).

### Synthesis of D01:

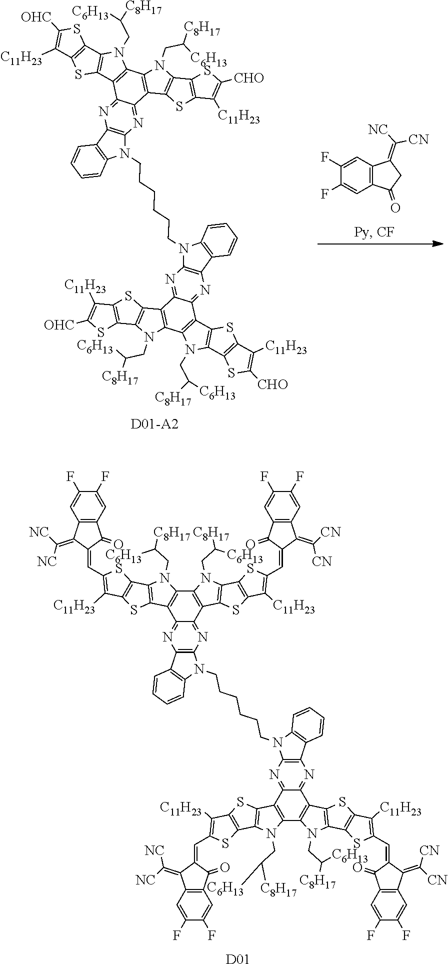

D01-A2 (0.11 g, 0.04 mmol), 5,6-Difluoro-1H-indene-1,3(2H)-dione (0.08 g, 0.34 mmol), and 7.0 mL of chloroform were placed into a double-necked flask under argon. 0.1 mL of pyridine was added in at room temperature and the reaction mixture was heated to 65° C. for 5 hours. After cooling to room temperature, 30 mL of methanol was added to precipitate the product. The solid was collected by filtration and purified by silica gel column (eluent by chloroform and heptane). The solid was precipitated with isopropyl alcohol to obtain blue-black solid product D01 (0.10 g, yield 75%). ¹H NMR (500 MHz, CDCl₃) δ 9.17 (s, 2H), 9.07 (s, 2H), 8.74-8.73 (m, 2H), 8.60-8.53 (m, 4H), 7.71 (t, J=8.0 Hz, 2H), 7.62-7.58 (m, 4H), 7.44-7.40 (m, 4H), 4.81-4.74 (m, 8H), 3.28-3.25 (m, 8H), 2.10-2.07 (m, 4H), 1.81-1.97 (m, 16H), 0.66-1.59 (m, 200H).

### Synthesis of S01:

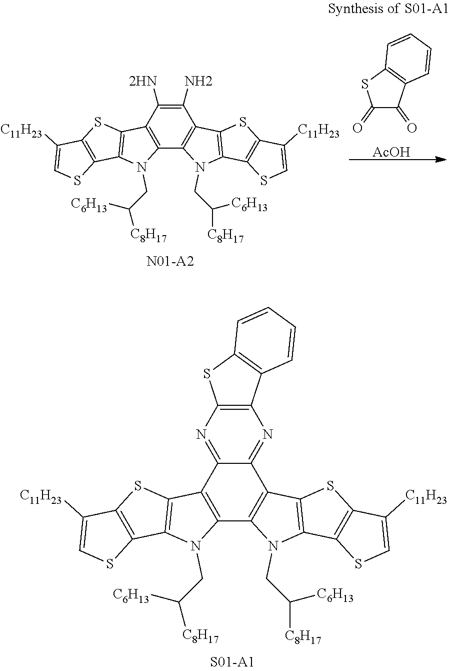

S01-A1 (0.27 g, yield 59%) was synthesized according to the procedure of N01-A3. N01-A2 (0.42 g, 0.36 mmol) and 1-benzothiophene-2,3-dione (0.06 g, 0.37 mmol) were used as starting materials. ¹H NMR (500 MHz, CDCl₃) δ 8.24 (d, J=8.5 Hz, 1H), 8.13 (d, J=7.5 Hz, 1H), 7.44 (dt, J=7.0, 1.0 Hz, 1H), 7.30 (dt, J=8.0, 1.0 Hz, 1H), 7.05 (s, 1H), 6.97 (s, 1H), 4.61 (d, J=7.5 Hz, 2H), 4.55 (d, J=7.5 Hz, 2H), 4.38-4.50 (m, 2H), 2.82 (t, J=7.5 Hz, 2H), 2.18 (t, J=7.5 Hz, 2H), 2.01-2.11 (m, 4H), 0.66-1.87 (m, 98H).

### Synthesis of S01-A2:

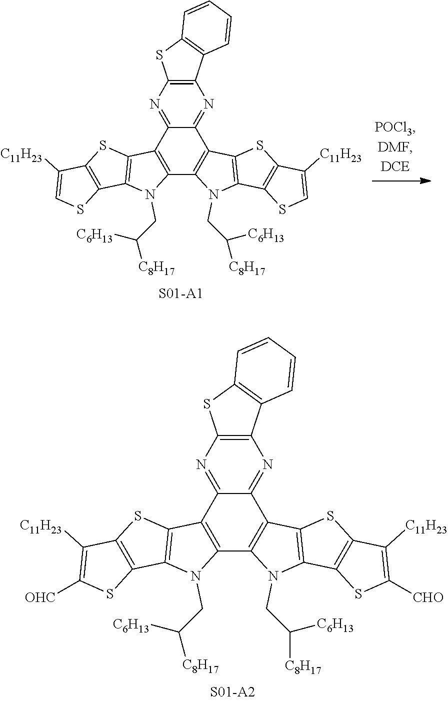

S01-A2 (0.16 g, yield 62%) was synthesized according to the procedure of N01-A4. S01-A1 (0.28 g, 0.19 mmol) was used as a starting material. ¹H NMR (500 MHz, CDCl₃) δ 10.16 (s, 1H), 10.09 (s, 1H), 8.14 (d, J=8.0 Hz, 1H), 8.09 (d, J=7.0 Hz, 1H), 7.39 (t, J=7.0 Hz, 1H), 7.30 (t, J=7.0 Hz, 1H), 4.64 (d, J=7.5 Hz, 2H), 4.58 (d, J=7.5 Hz, 2H), 3.21 (t, J=8.0 Hz, 2H), 3.04 (t, J=7.5 Hz, 2H), 2.07-1.98 (m, 2H), 1.96-0.66 (m, 102H).

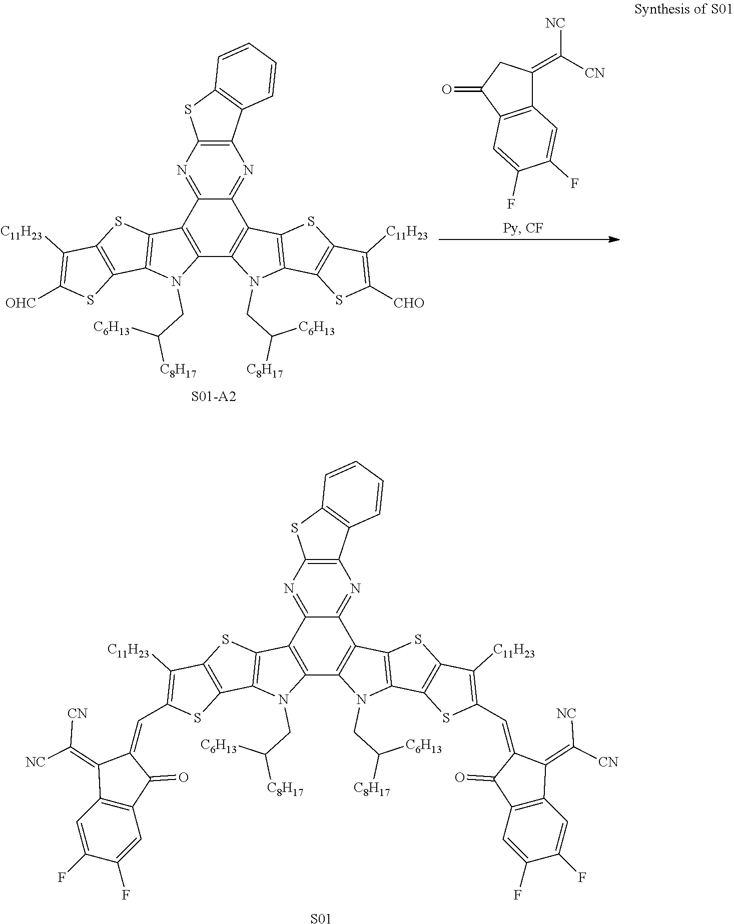

A black solid product S01 (0.05 g, yield 24%) was synthesized according to the procedure of N01. S01-A2 (0.16 g, 0.12 mmol) was used as a starting material. ¹H NMR (500 MHz, CDCl₃) δ 9.18 (s, 1H), 9.16 (s, 1H), 8.57 (m, 2H), 8.10 (d, J=8.0 Hz, 1H), 8.02 (m, 1H), 7.69 (m, 2H), 7.32 (m, 1H), 7.08 (m, 1H), 4.78-4.65 (m, 4H), 3.29-3.25 (m 4H), 2.17-0.66 (m, 104H).

### Synthesis of P01:

### Synthesis of P01-I0:

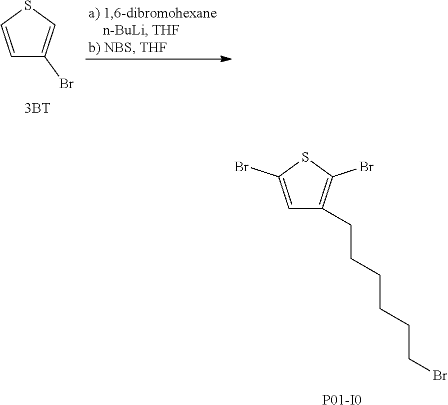 [SMILES: Brc1ccsc1 ; BrCCCCCCc1cc(Br)sc1Br ; "P01-I0"]

Reference J. Mater. Chem. 2011, 21, 2733-2739 synthesis method to prepare P01-10.

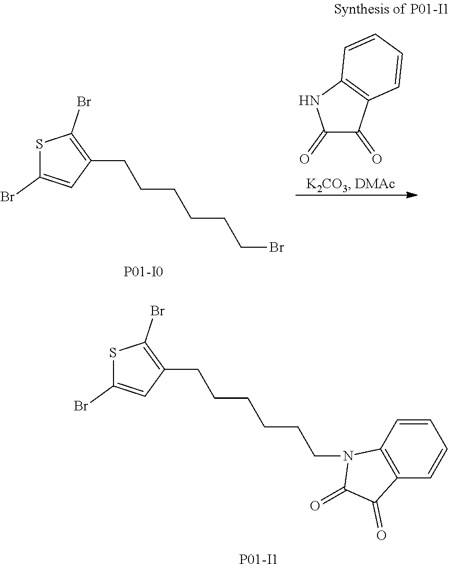

Isatin (0.50 g, 3.40 mmol) was dissolved in 10 mL of dimethylacetamide under nitrogen. Potassium carbonate (1.88 g, 13.59 mmol) was added and stirred at room temperature for 30 minutes. P01-I0 (1.51 g, 3.74 mmol) and 10 mL of dimethylacetamide were slowly added, and the mixture was heated to 85° C. for 20 hours. After cooling, quenched with 20 mL of deionized water, and extracted with dichloromethane. The reaction mixture was dried with magnesium sulfate and concentrate to obtain crude product. The crude product was purified by silica gel column (the eluent was methylene chloride) to obtain P01-I1 (1.24 g, yield 78%). ¹H NMR (600 MHz, CDCl₃) δ 7.61-7.57 (m, 2H), 7.11 (t, J=7.8 Hz, 1H), 6.88 (d, J=7.8 Hz, 1H), 6.75 (s, 1H), 3.71 (t, J=7.2 Hz, 2H), 2.50 (t, J=7.2 Hz, 2H), 1.73-1.68 (m, 2H), 1.57-1.52 (m, 2H), 1.42-1.35 (m, 4H).

### Synthesis of P01-A1:

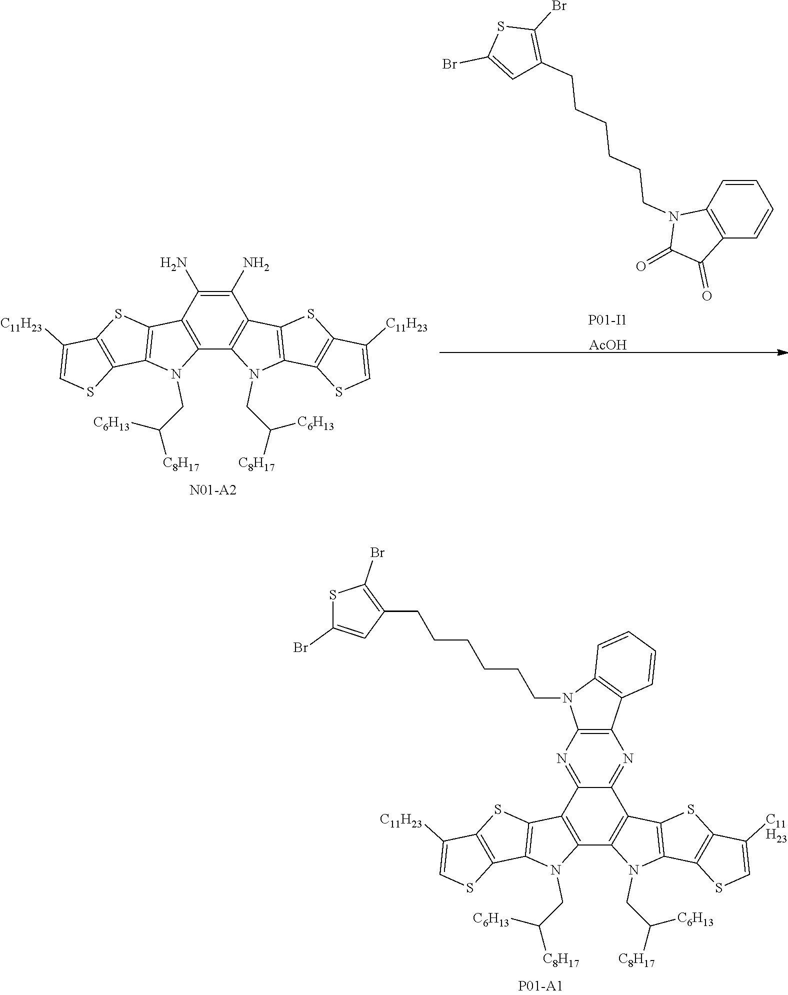

P01-A1 (0.52 g, yield 42%) was synthesized according to the procedure of N01-A3. N01-A2 (0.9 g, 0.77 mmol) and P01-I1 (0.38 g, 0.81 mmol) were used as starting materials. ¹H NMR (600 MHz, CDCl₃) δ 8.79 (d, J=7.2 Hz, 1H), 7.61 (dt, J=7.8, 1.0 Hz, 1H), 7.42 (t, J=7.2 Hz, 1H), 7.38 (d, J=8.4 Hz, 1H), 7.00 (s, 1H), 6.97 (s, 1H), 6.75 (s, 1H), 4.61-4.52 (m, 6H), 4.55 (d, J=7.5 Hz, 2H), 2.87-2.83 (m, 4H), 2.56 (t, J=7.2 Hz, 2H), 1.98-1.87 (m, 8H), 1.67-0.67 (m, 102H).

### Synthesis of P01-A2:

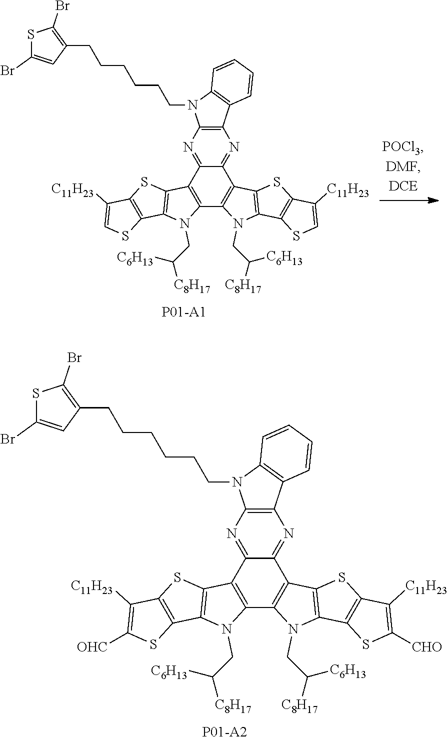

P01-A2 (0.49 g, yield 91%) was synthesized according to the procedure of N01-4. P01-A1 (0.52 g, 0.32 mmol) was used as a starting material. ¹H NMR (500 MHz, CDCl₃) δ 10.15 (s, 1H), 10.14 (s, 1H), 8.79 (dd, J=6.5, 1.0 Hz, 1H), 7.67 (dt, J=6.5, 1.0 Hz, 1H), 7.46 (t, J=6.5 Hz, 1H), 7.42 (d, J=8.0 Hz, 1H), 6.75 (s, 1H), 4.64-4.53 (m, 6H), 3.23 (t, J=6.5 Hz, 4H), 2.55 (t, J=6.5 Hz, 2H), 2.00-1.93 (m, 6H), 1.87-1.81 (m, 2H), 1.96-0.66 (m, 102H).

### Synthesis of P01-A3:

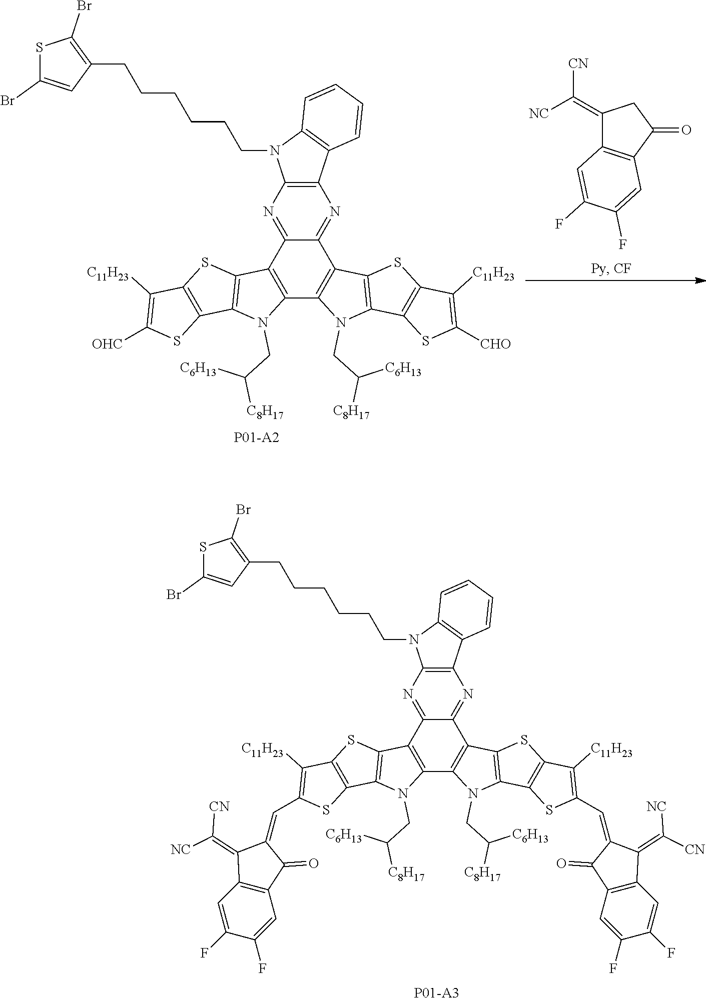

P01-A3 (0.56 g, yield 92%) was synthesized according to the procedure of N01. P01-A2 (0.49 g, 0.29 mmol) was used as a starting material. ¹H NMR (600 MHz, CDCl₃) δ 9.18 (s, 2H), 8.79 (d, J=7.8 Hz, 2H), 8.59-8.56 (m, 2H), 7.72-7.67 (m, 2H), 7.49 (t, J=8.4 Hz, 1H), 7.42 (d, J=8.4 Hz, 1H), 6.75 (s, 1H), 4.77-4.54 (m, 6H), 3.30-3.27 (m, 4H), 2.55 (t, J=9.0 Hz, 2H), 1.99-0.65 (m, 112H).

### Synthesis of P01:

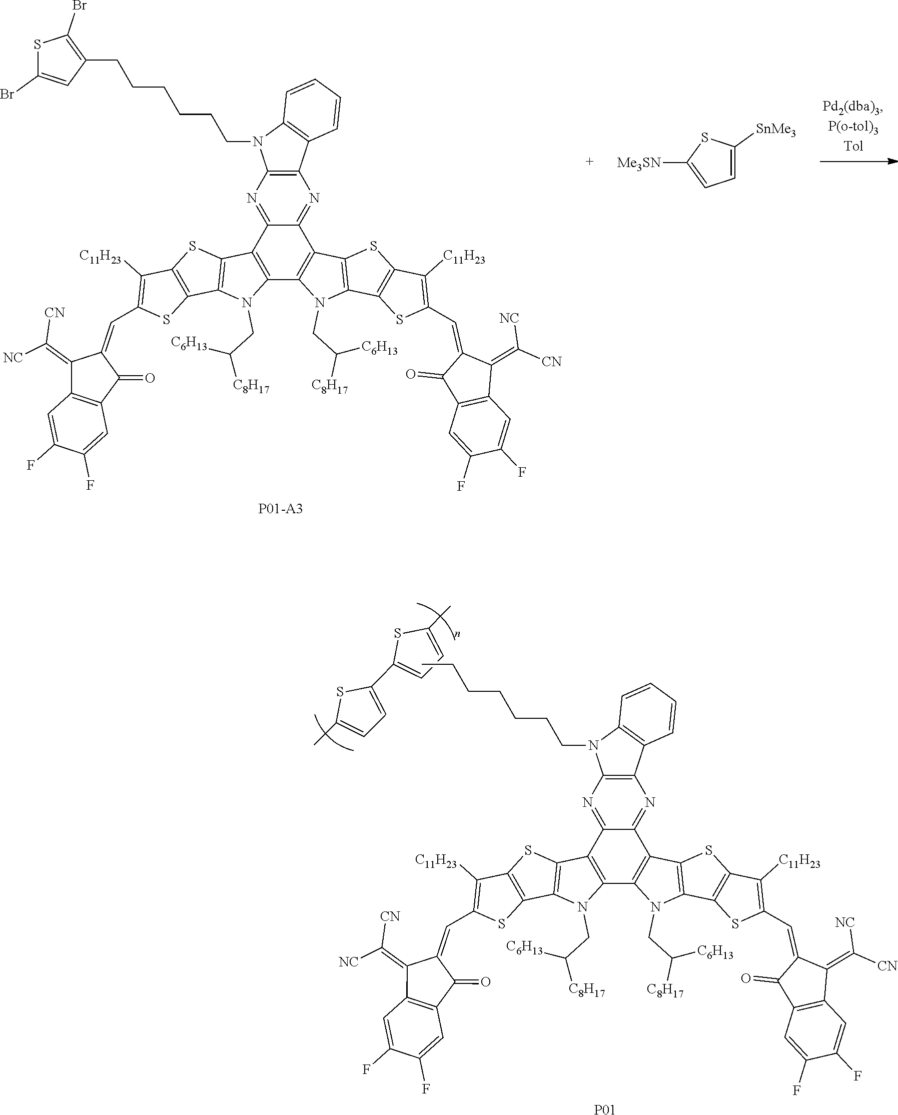

P01-A3 (0.21 g, 0.10 mmol), 2,5-trimethylstannylthieno (0.04 g, 0.10 mmol), tris(dibenzylideneacetone) dipalladium (0.01 g, 0.001 mmol) and tris(o-methylphenyl)phosphine (0.012 g, 0.004 mmol) were placed in a 100 mL two necked flask. 6 mL of deoxygenated toluene was injected into the mixture under argon. The reaction mixture was heated to 90° C. for 45 minutes. The reaction was terminated with 2-bromothiophene. The reaction mixture was cooled to room temperature and filtered through celite, and then precipitated in methanol to obtain a black crude product. The crude product was purified by Soxhlet extraction with methanol and acetone to obtain black solid product P01 (0.13 g, yield 64%).

### Characteristic Testing of Organic Optoelectronic Compounds:

Please refer to Table 1, which shows the test results of Examples N01, N03, N05, N14, N15, D01, S01, P01 and Comparative Example Ref.

TABLE 1
Test results of Examples N01, N03, N05, N14, N15,
D01, S01, P01 and Comparative Example Ref.

| Material | Eg | HOMO/LUMO | Absorption | | ε ^{a} |
|---|---|---|---|---|---|
| Properties | (eV) | (eV) | film | solution | (M⁻¹cm⁻¹) |
| N01 | 1.42 | −5.64/−4.22 | 829 | 730 | 185200 |
| N03 | 1.41 | −5.55/−4.14 | 807 | 749 | 114700 |
| N05 | 1.42 | −5.63/−4.21 | 825 | 756 | 192100 |
| N14 | 1.41 | −5.57/−4.16 | 806 | 749 | 107500 |
| N15 | 1.40 | −5.61/−4.21 | 804 | 750 | 245900 |
| D01 | 1.37 | −5.60/−4.23 | 820 | 748 | 431700 |
| S01 | 1.42 | −5.56/−4.14 | 794 | 721 | 121800 |
| P01 | 1.40 | −5.55/−4.15 | 812 | 758 | 123100 |
| Ref. | 1.35 | −5.72/−4.37 | 822 | 731 | 186000 |

^{a} The ε is measure in chloroform with 1 × 10⁻⁵ M concentration.

Wherein, the structure of Comparative Example Ref. is:

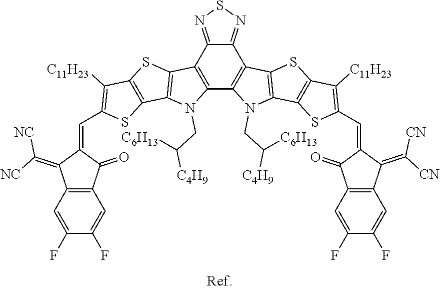

The examples and Comparative Example Ref. are all ladder-shaped molecules formed by the A-D-A′-D-A structure. However, the difference is that the organic optoelectronic compound of the present invention has a pyrazine core structure. The examples and Comparative Example Ref. listed in the table all use cyclic voltammetry to measure the oxidation properties. After calculation (HOMO=−|4.71+E^{ox}−E^{ferroncene}|), the highest occupied molecular orbital (HOMO) is obtained, and then through the absorption starting position (λ_{film}^{onset}) of the UV-Vis-NIR absorption spectrum of the material's thin film state, the optical energy gap (E_{g}=1241/λ_{film}^{onset}) and the lowest unoccupied molecular orbital (LUMO) of the material can be known, LUMO=HOMO+E_{g}). As shown in Table 1, energy level, HOMO, LUMO, and the maximum absorption value and absorption coefficient of film state and solution state are recorded.

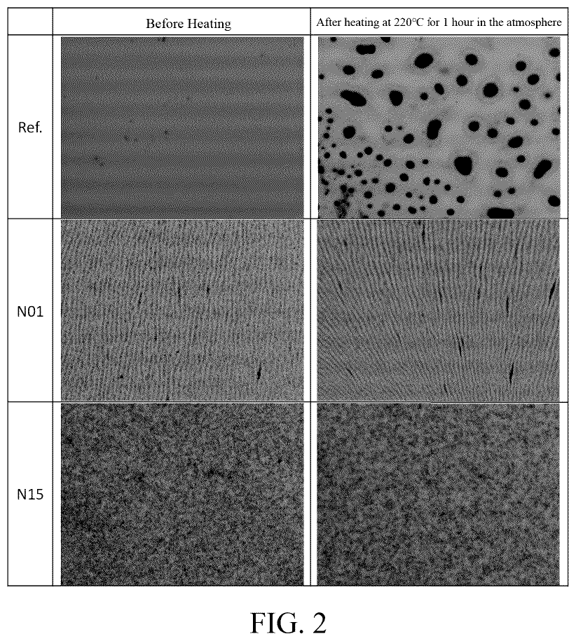

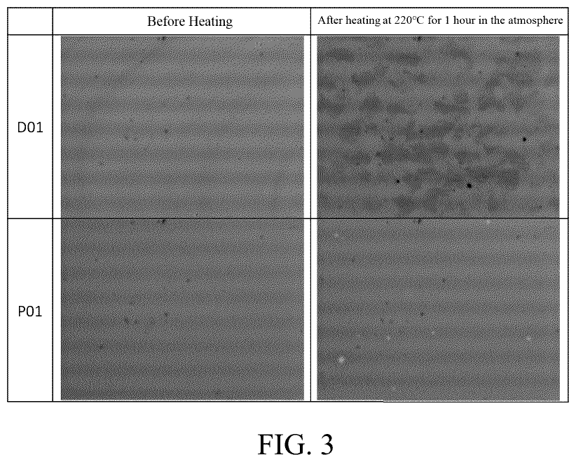

Please refer to FIG. 2 and FIG. 3. FIG. 2 shows thermal stability test results of film surfaces of a Comparative Example Ref., and embodiments of N01 and N15 of the present invention. FIG. 3 shows thermal stability test results of film surfaces of embodiments of D01 and P01 of the present invention. In order to test the thermal stability performance of the examples and compare them with the Comparative Example Ref., the Comparative Examples Ref., Examples N01, N15, D01 and P01 were coated into thin film samples, and observed the initial film surface before heating and the film surface after heating at 220° C. for 1 hour in the atmosphere. As shown in FIG. 2 and FIG. 3, the film surface of Comparative Example Ref. agglomerated after heating, while the film surface of the Examples is not changed. It can be seen from this that the thermal stability of the organic optoelectronic compounds of the present invention is better than that of the Comparative Example Ref.

### Preparation and Thermal Stability Performance Testing of Organic Photodetectors of Organic Optoelectronic Devices:

A glass coated by a pre-patterned indium tin oxides (ITO) with a sheet resistance of ˜15 Ω/sq is used as a substrate. The substrate is ultrasonically oscillated in soap deionized water, deionized water, acetone, and isopropanol in sequence, and washed in each step for 15 minutes. The washed substrate is further treated with a UV-ozone cleaner for 15 minutes. The top coat of AZO (Aluminum-doped zinc oxide) solution is spin coated on the ITO substrate with a spin rate of 2000 rpm for 40 seconds, and then baked at 120° C. in air for 5 minutes to form an electron transporting layer (ETL). The active layer solution was prepared with the weight ratio of donor material:acceptor material is 1:1˜2). The donor material includes DO-01 to DO-34 materials, and the acceptor material includes at least one of the organic optoelectronic compounds and the active layer material of the present invention. In order to completely dissolve the active layer material, the active layer solution needs to be stirred on a hot plate at 100° C. for at least 3 hours. After completely dissolving the active layer material, the active layer solution is filtered with PTFE filter membrane (pore size 0.45˜1.2 μm) and heated for 1 hour. Then, the active layer solution is cooled to the room temperature for spin coating, and the spin rate was used to control the film thickness in the range of 100˜800 nm. Finally, the thin film formed by the coated active layer is annealed at 100° C. for 5 minutes, and then transferred to a thermal evaporation machine. A thin layer (8 nm) of MoO₃ is deposited as a hole transporting layer (HTL) under a vacuum of 3×10⁻⁶ Torr. In this experiment, a Keithley™ 2400 source meter was used to record the dark current (J_{d}, at a bias of 0.5˜−6.0 V) in the absence of light. External quantum efficiency system was used to measure external quantum efficiency (EQE) with a range of 300˜1100 nm (bias voltage 0˜−0.5 V), and silicon (300˜1100 nm) is used for light source calibration. The component heat resistance test is to place the organic optoelectronic device on a hot plate with 160° C. and heat it for 1 hour, then return it to room temperature for efficiency measurement.

It should be noted here that, in practical applications, the first electrode preferably has good light transmittance. The first electrode is usually made of a transparent conductive material, preferably selected from one of the following conductive material groups: indium oxide, tin oxide, fluorine-doped tin oxide derivative (Fluorine Doped Tin Oxide, FTO), or composite metal oxides such as indium tin oxide (ITO) and indium zinc oxide (IZO). The material of the second electrode is a conductive metal, preferably silver or aluminum, more preferably silver. Suitable and preferred materials for electron transporting layer include, but are not limited to, metal oxides such as ZnOₓ, aluminum doped ZnO (AZO), TiOₓ or nanoparticles thereof, salts (such as LiF, NaF, CsF, CsCO₃), amines (such as primary amines, secondary or tertiary amines), conjugated polymer electrolytes (such as polyethyleneimine), conjugated polymers (such as poly[3-(6-trimethylammoniumhexyl)thiophene], poly(9,9)-bis(2-ethylhexyl-fluorene)-b-poly[3-(6-trimethylammoniumhexyl)thiophene] or poly[(9,9-bis(3′-(N,N-dimethylamino))propyl)-2,7-fluorene)-alt-2,7-(9,9-dioctylfluorene)], and organic compounds such as tris(8-quinolinyl)-aluminum (III) (Alq₃), 4,7-diphenyl-1,10-phenanthroline, or a combination of one or more of the foregoing. Suitable and preferred materials for hole transporting layer include, but are not limited to metal oxides such as ZTO (Zinc Tin Oxide), MoOₓ, WOₓ, NiOₓ, SnOₓ or nanoparticles thereof, metal-containing salts, such as copper sulfide, copper thiocyanate, copper iodide, copper indium sulfide, lead sulfide, cobalt acetate, tungsten disulfide, etc., conjugated polymer electrolytes such as PEDOT:PSS, polymeric acids such as polyacrylates, conjugated polymers such as polytriarylamine (PTAA), insulating polymers such as Nafion films, polyethyleneimine or polystyrene sulfonates, organic compounds such as N,N′-diphenyl-N,N′-bis(1-naphthyl) (1,1′-biphenyl)-4,4′-diamine (NPB), N,N′-diphenyl-N,N′-(3-methylbenzene base)-1,1′-biphenyl-4,4′-diamine (TPD), or a combination of one or more of the above.

Please refer to Table 2. Table 2 shows the thermal stability test results of organic optoelectronic devices Examples 1 to 4 and Comparative Example Ref. Wherein, the donor material of the organic optoelectronic devices is DO-34, and the acceptor materials are organic optoelectronic compounds N14, N15, D01, P01 and Ref. It should be understood that although the following tests use DO-34 as the donor material, it is not limited to this. This is only an exemplary explanation. The DO-01˜DO-33 proposed above can also have similar test results. In addition, in the following tests, N14 and N15 are selected to represent small molecules, D01 is selected to represent dimers, and P01 is selected to represent polymer. However, this is not a limitation. This is only an exemplary explanation. The above-mentioned embodiments are also Similar test results are possible.

Table 2 shows the thermal stability test results of organic optoelectronic devices Examples 1 to 4 and Comparative Example Ref.

EQE⁸⁰⁰ Retension (−4 V)

| OPD | | Pristine | 1 h@160° C. |
|---|---|---|---|
| Device | ATL | (a.u.) | (a.u.) |
| 1 | DO-34: N14 | 1 | 0.88 |
| 2 | DO-34: N15 | 1 | 1.16 |
| 3 | DO-34: D01 | 1 | 0.69 |
| 4 | DO-34: P01 | 1 | 1.10 |
| Ref. | DO-34: Ref | 1 | 0.22 |

As shown in Table 2, Table 2 shows the EQE performance of the tested organic optoelectronic devices before heating and after heating to 160° C. for 1 hour. The EQE before heating is set to 1 to compare the results of the EQE tested after heating. It can be seen from Table 2 that the difference between the EQE of the heated organic optoelectronic devices of the present invention and the EQE of the unheated one is approximately ±0.2. Even though the thermal stability of Example 3 is worse than that of Examples 1 to 4, it can still maintain about 70% of EQE efficiency. In comparison, the EQE result of the Comparative Example Ref. is only 20%. The organic optoelectronic devices of the present invention have good thermal stability. The thermal stability test in Table 2 echoes the thermal stability test results of the film surface in FIG. 2 and FIG. 3. The organic optoelectronic compound of the present invention and the organic optoelectronic device using the same have good thermal stability.

Based on the above experimental results, the organic optoelectronic compound of the present invention has a pyrazine core structure and can be used as a non-fullerene acceptor material. Wherein, the structure of Formula 1 has the following characteristics: (1) providing additional conjugation planes for molecules and strengthening the interaction between molecules, which makes carrier flow smoother and reduces energy loss; in addition, the arrangement and energy level of the material can also be changed by changing the functional groups and structural symmetry, and the asymmetric structure has the effect of reducing energy dissipation through non-radiation; (2) the added structure can extend the conjugation and increase the structural rigidity to enhance the thermal stability of the material and, in addition, the addition of heteroaromatic rings can also be used to synthesize dimers or trimers to further improve thermal stability and have the opportunity to extend the material stacking network; and (3) since adding a conjugated plane may cause the problem of decreased solubility of the material, the material modified in this way can modify the functional groups in a simpler way and can change the material's properties without affecting the overall conjugated system too much solubility and clustering patterns.

With the detailed description of the above embodiments, it is hoped that the features and spirit of the present invention can be more clearly described, and the scoped of the present invention is not limited by the embodiments disclosed above. On the contrary, the intention is to cover various changes and equivalent arrangements within the scope of the patents to be applied for in the present invention.

What is claimed is:

1. An organic optoelectronic compound comprises a structure such as Formula I: 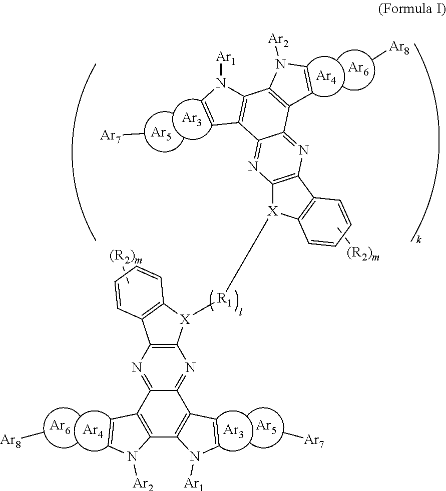
wherein R₁ is selected from the following groups and their derivatives consisting of C1˜C30 alkyl, C6˜C30 aryl, C3˜C30 heteroaryl, C1˜C8 haloalkyl, C4˜C30 aralkyl, C4˜C30 alkoxy and polymer chain;
R₂ and R₁ can be the same or different, R₂ is selected from the following groups and their derivatives consisting of C1˜C30 alkyl, C6˜C30 aryl, C3˜C30 heteroaryl, C1˜C8 haloalkyl, C4˜C30 aralkyl, C4˜C30 alkoxy, hydrogen, fluorine, chlorine and bromine;
Ar₁ and Ar₂ can be the same or different, and are independently selected from the following groups and their derivatives consisting of C6˜C30 carbon chain, C6˜C30 cycloalkyl, C6˜C30 aryl, C2˜C30 heteroaryl, C1˜C30 alkoxy and C7˜C30 phenalkyl;
Ar₃ to Ar₆ can be the same or different, and are independently selected from the following groups and their derivatives consisting of C6˜C30 aryl, C2˜C30 heteroaryl and C2˜C8 spiro;
Ar₇ and Ar₈ can be the same or different electron-withdrawing groups, and each of the electron-withdrawing groups is a polycyclic structure, with or without substituents, comprising at least one five-membered ring and at least one six-membered ring; a polycyclic structure, with or without substituents, comprising at least two five-membered rings; or a polycyclic structure, with or without substituents, comprising at least two six-membered rings; and
X is selected from the following group consisting of nitrogen, oxygen, sulfur and selenium;
wherein, when X is nitrogen, k≥0, l is an integer from 1 to 10, and m is an integer from 0 to 10; and
when X is oxygen, sulfur, or selenium, k and l are 0, and m is an integer from 0 to 10.

2. The organic optoelectronic compound of claim 1, wherein when X is nitrogen and R₁ is a non-polymer chain, k is an integer from 0 to 2, and 1 is an integer from 1 to 10, and m is an integer between 0 and 10.

3. The organic optoelectronic compound of claim 1, wherein when X is nitrogen and R₁ is a polymer chain, k≥0, l is 1, and m is an integer from 0 to 10.

4. The organic optoelectronic compound of claim 1, wherein Ar₁ and Ar₂ are independently selected from the following structures: 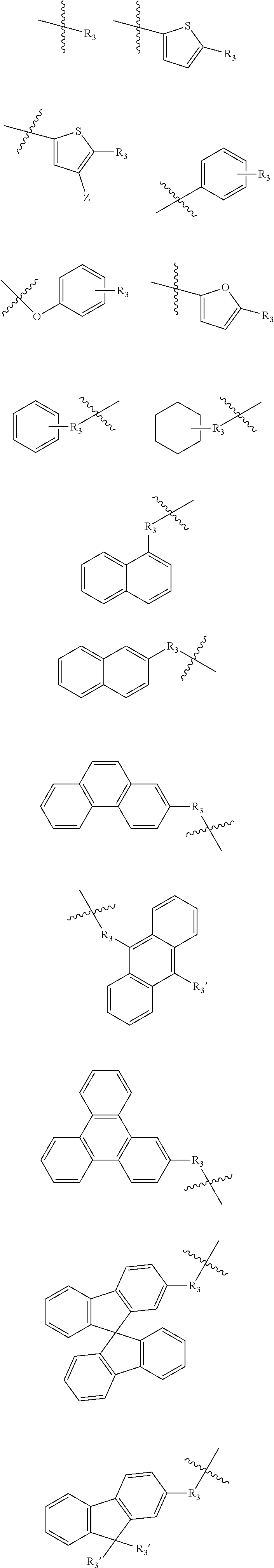
wherein, Z comprises halogen; R₃ is selected from the following groups and their derivatives consisting of C1˜C30 linear alkyl, C1˜C30 branched alkyl and C1˜C30 alkoxy; and
R₃′ is selected from the following groups and their derivatives consisting of C1˜C30 linear alkyl, C1˜C30 branched alkyl, C6˜C30 aryl, C2˜C30 heterocycle, C1˜C30 alkoxy and C7˜C30 phenalkyl.

5. The organic optoelectronic compound of claim 1, wherein Ar₃ and Ar₄ are independently selected from the following structures: 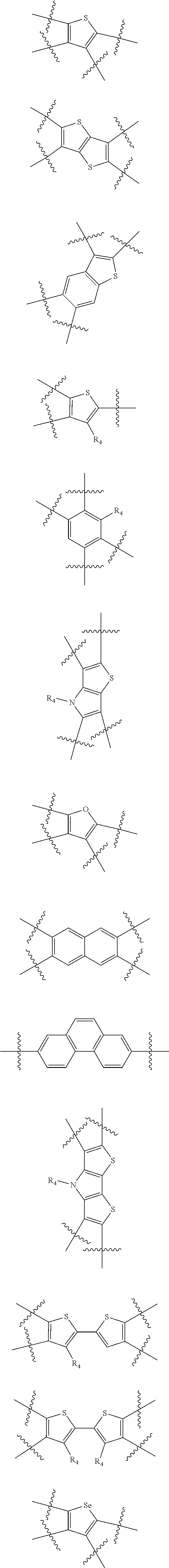
wherein, R₄ is selected from the following groups and their derivatives consisting of C1˜C30 alkyl, C2˜C30 heterocycle, C1˜C30 alkoxy and C7˜C30 phenalkyl.

6. The organic optoelectronic compound of claim 1, wherein Ar₅ and Ar₆ are independently selected from the following structures: 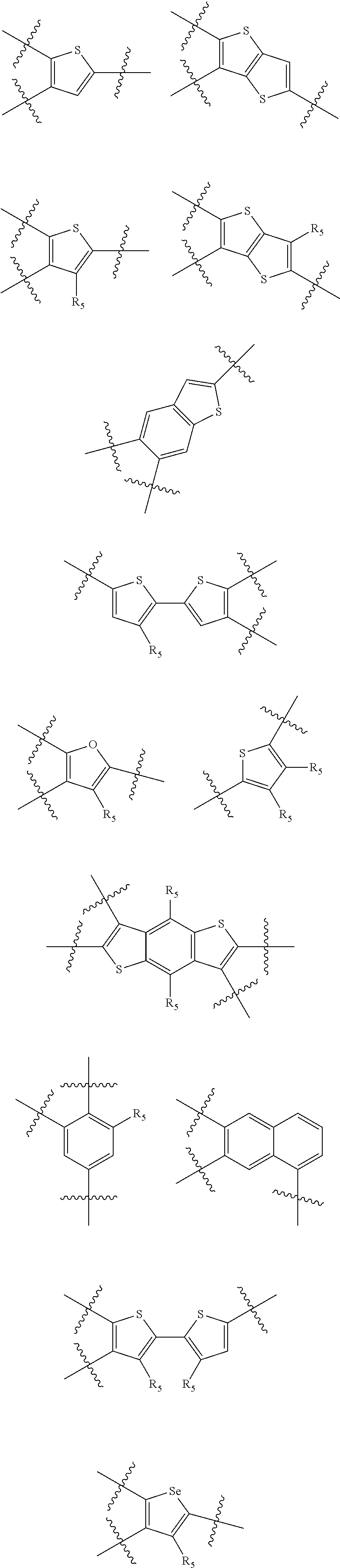
wherein, R₅ is selected from the following groups and their derivatives consisting of C1˜C30 alkyl, C6˜C30 aryl, C2˜C30 heterocycle, C1˜C30 alkoxy, C7˜C30 phenalkyl, hydrogen, fluorine and chlorine.

7. The organic optoelectronic compound of claim 1, wherein the polycyclic structure of Ar₇ and Ar₈ comprises at least one of the following two: at least one of the five-membered rings comprises at least one of cyano and C═O, and at least one of the six-membered rings comprises at least one of hydrogen, halogen and C═O as a substituent.

8. An active layer material comprising:
an acceptor material comprising an organic optoelectronic compound of claim 1; and
a donor material comprising at least one organic conjugated polymer.

9. The active layer material of claim 8, wherein the organic conjugated polymer is selected from the following structures: 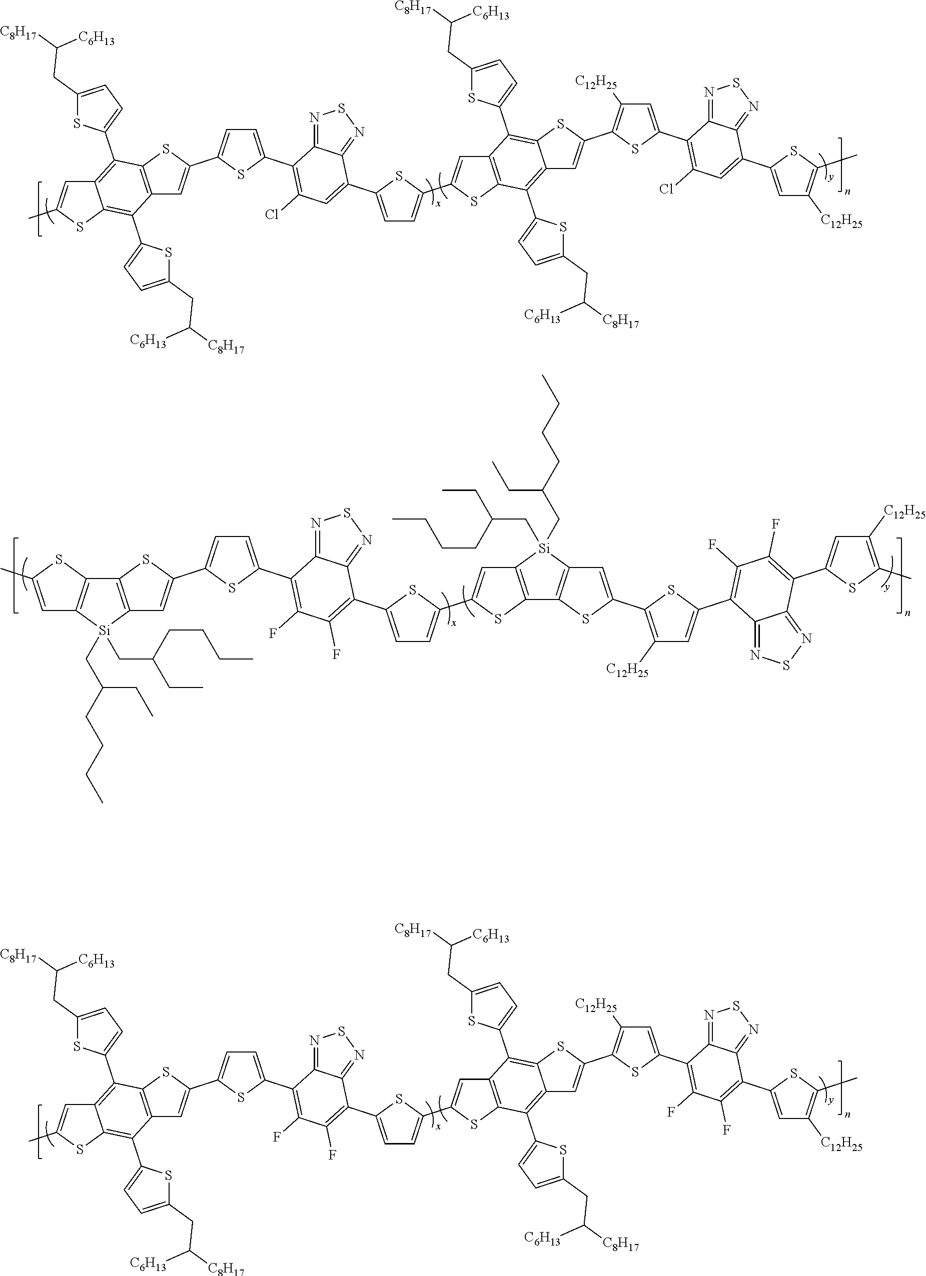 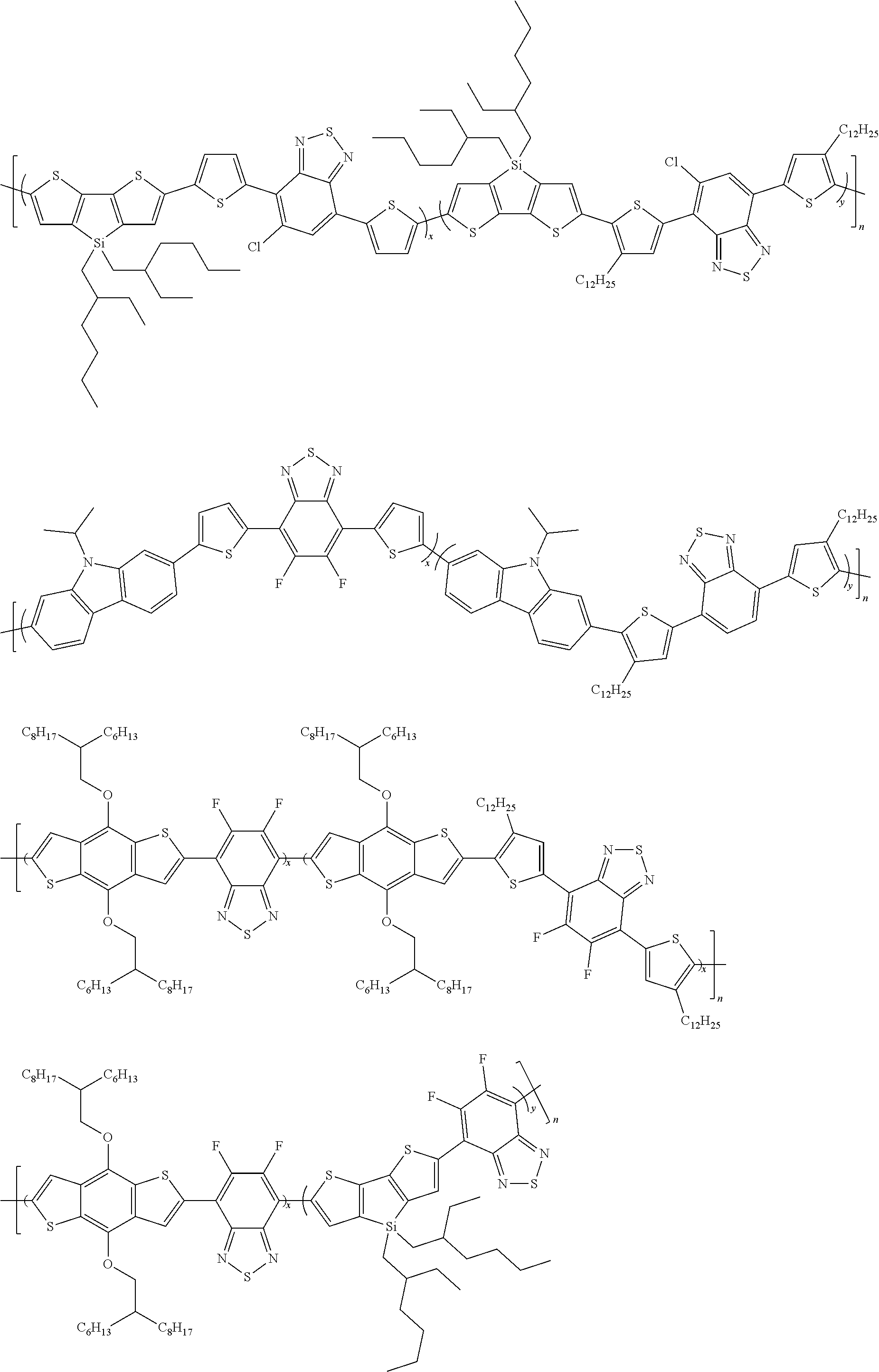 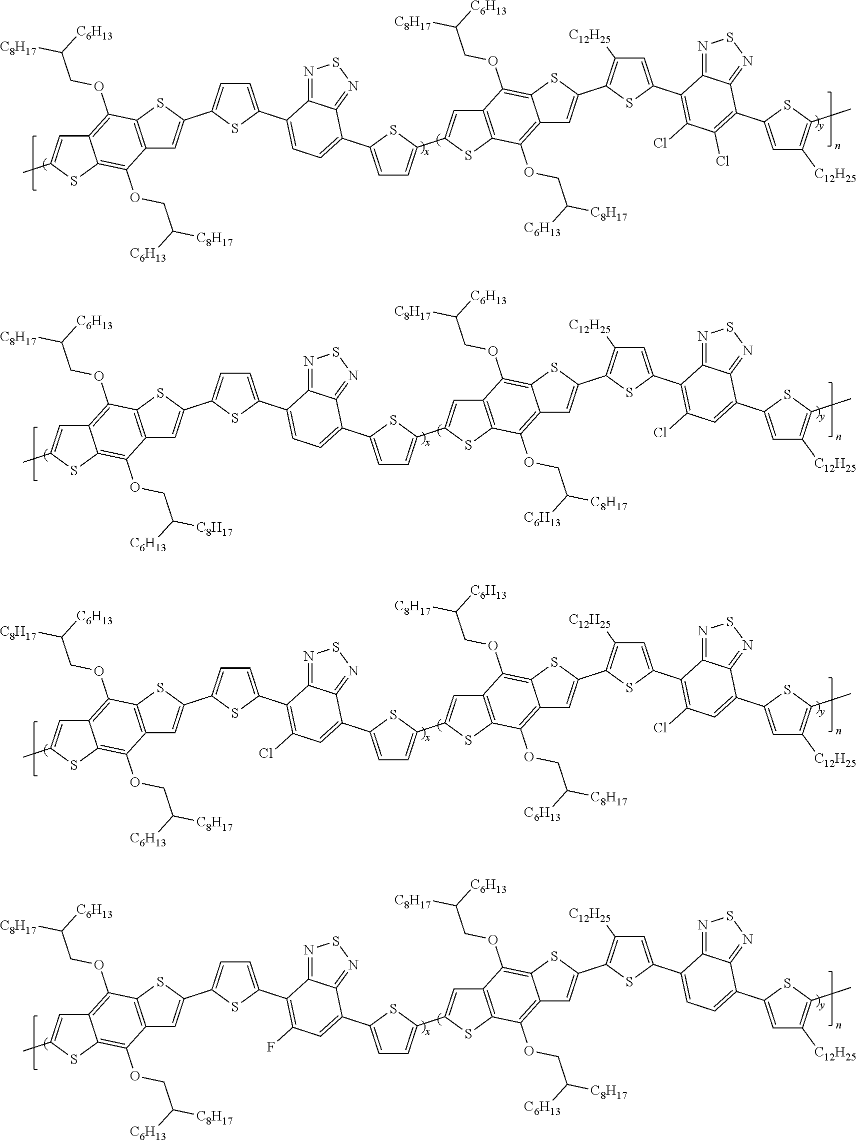 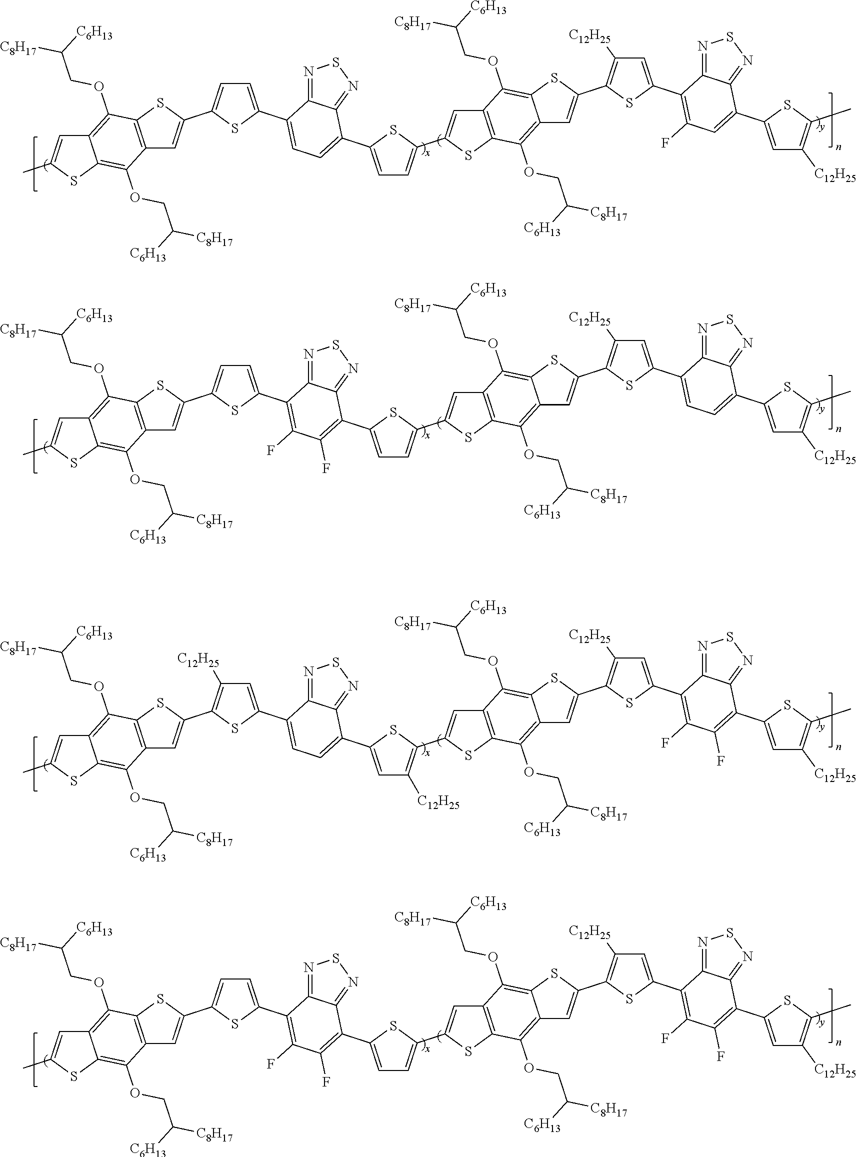 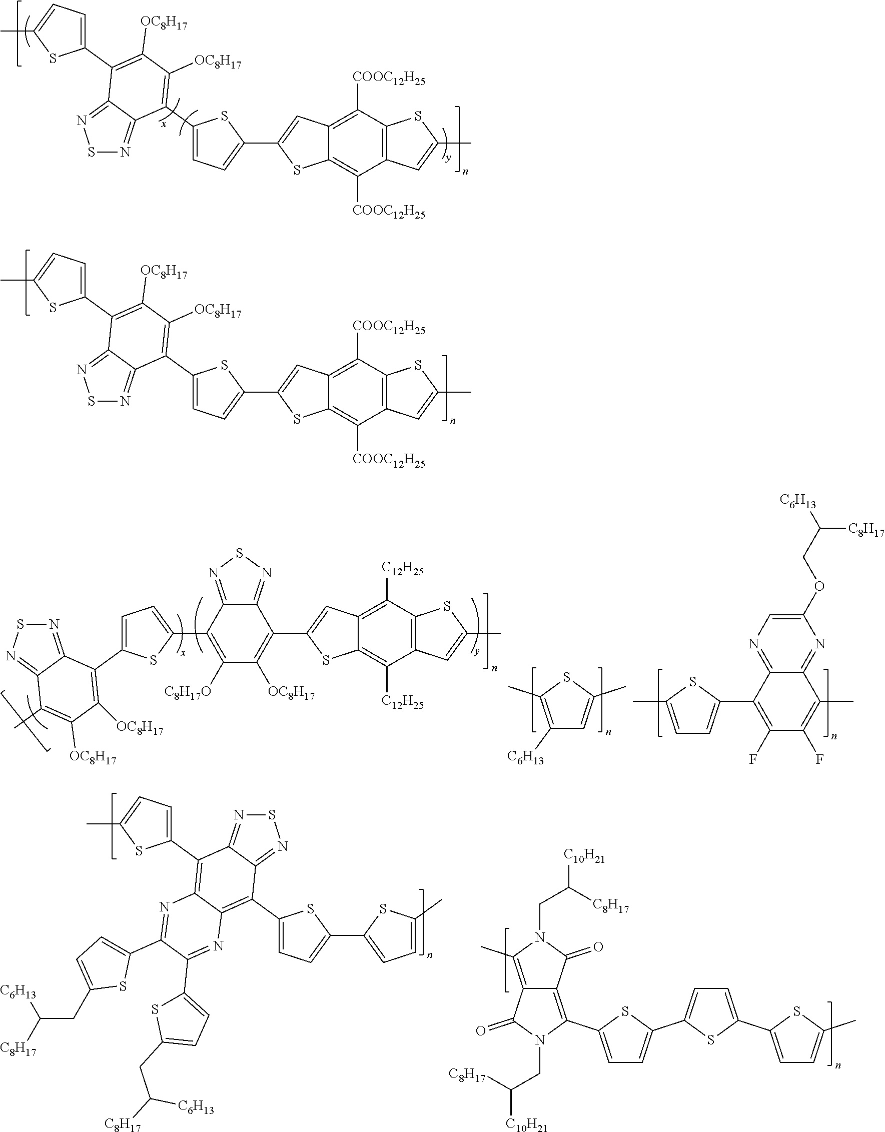 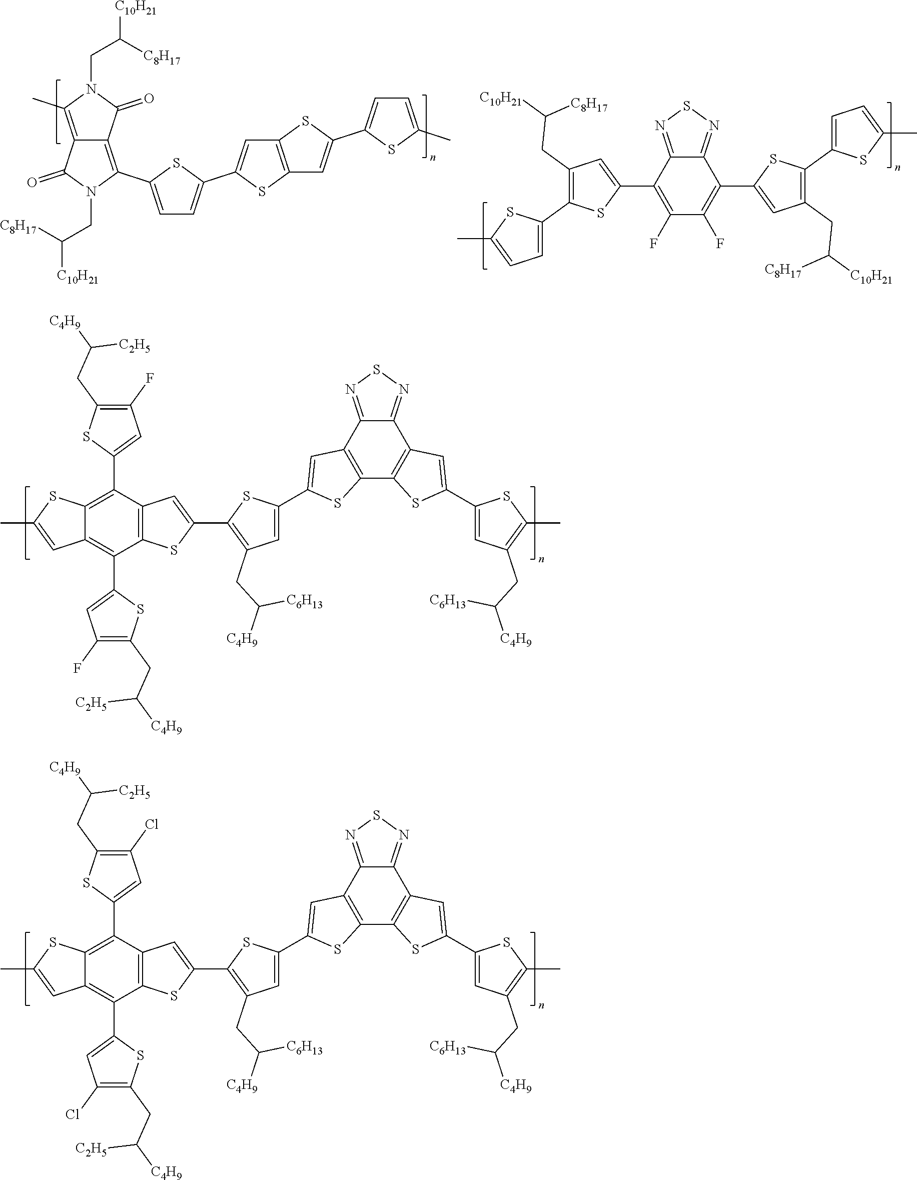 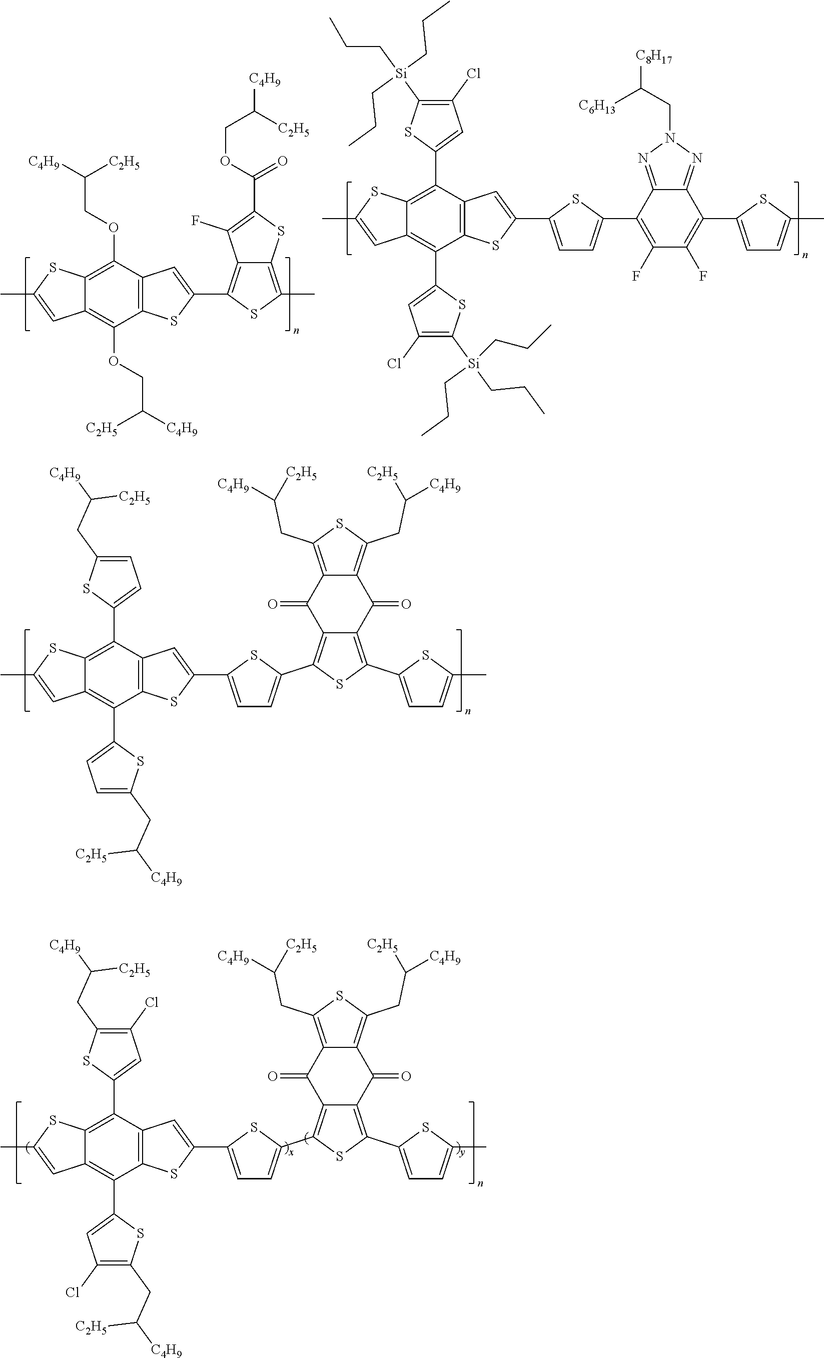 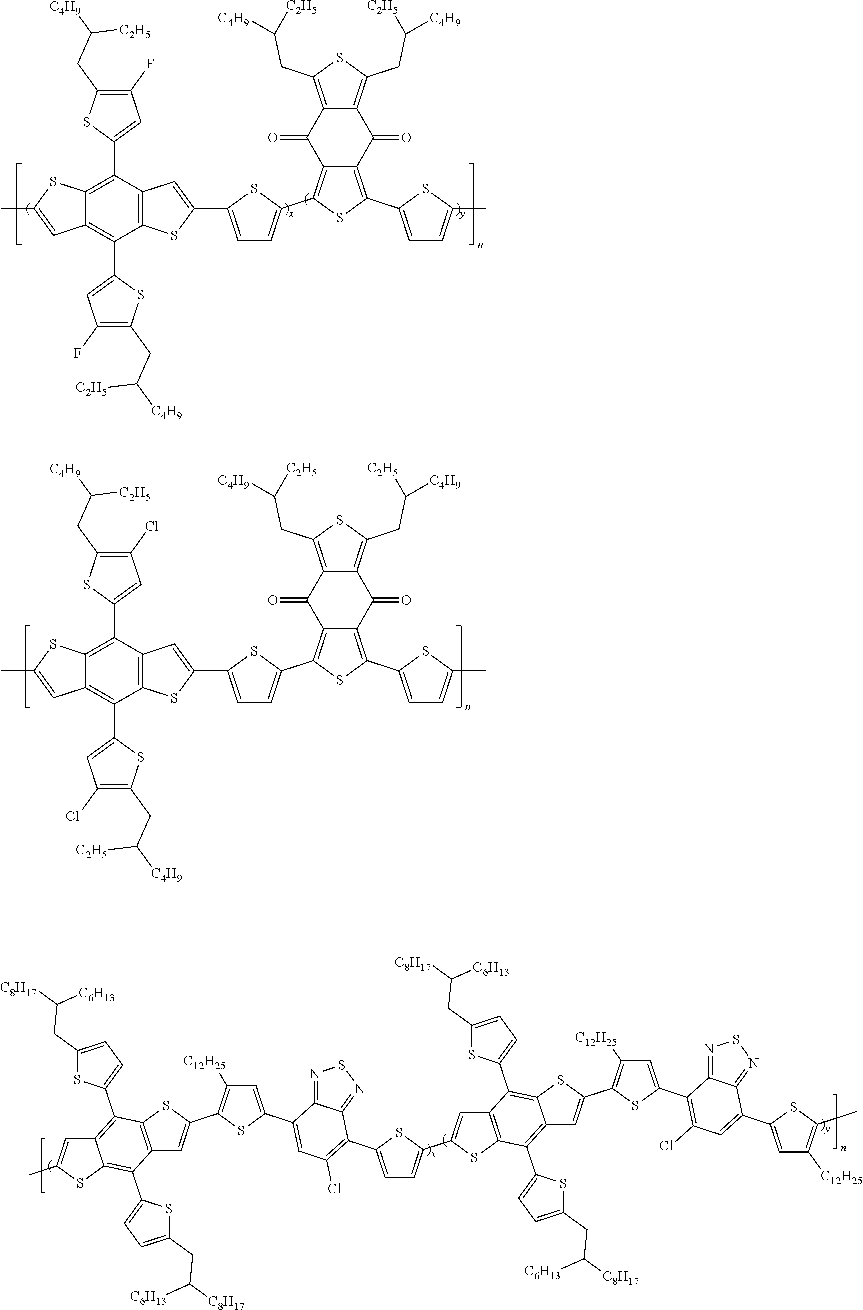 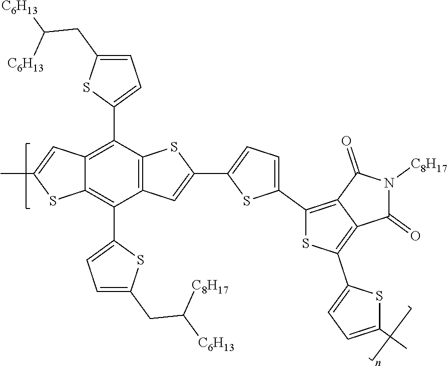
wherein, 0<x<1, 0<y<1, and x+y=1; and n is a positive integer greater than 1.

10. An organic optoelectronic device comprising:
a first electrode;
an active layer comprising at least one of an organic optoelectronic compound of claim 1; and
a second electrode, wherein the active layer is disposed between the first electrode and the second electrode, and at least one of the first electrode and the second electrode is a transparent or semi-transparent electrode.

11. The organic optoelectronic device of claim 10, further comprising a first carrier transporting layer and a second carrier transporting layer, wherein the first carrier transporting layer is disposed between the first electrode and the active layer and the first carrier transporting layer is configured to transport carriers in the first electrode and the active layer; the second carrier transporting layer is disposed between the active layer and the second electrode and the second carrier transporting layer is configured to transport carriers in the active layer and the second electrode.

12. An organic optoelectronic device comprising:
a first electrode;
an active layer comprising at least one of an active layer material of claim 8; and
a second electrode, wherein the active layer is disposed between the first electrode and the second electrode, and at least one of the first electrode and the second electrode is a transparent or semi-transparent electrode.

13. The organic optoelectronic device of claim 12, further comprising a first carrier transporting layer and a second carrier transporting layer, wherein the first carrier transporting layer is disposed between the first electrode and the active layer and the first carrier transporting layer is configured to transport carriers in the first electrode and the active layer; the second carrier transporting layer is disposed between the active layer and the second electrode and the second carrier transporting layer is configured to transport carriers in the active layer and the second electrode.
